(19) United States
(12) Patent Application Publication
(10) Pub. No.: US 2025/0136703 A1
(43) Pub. Date: May 1, 2025

(54) ANTI-IL13RA2 ANTIBODIES AND USES THEREOF

(71) Applicant: Phanes Therapeutics, Inc., San Diego, CA (US)

(72) Inventors: Huiwen Wu, San Diego, CA (US); Haiqun Jia, San Diego, CA (US); Hui Zou, San Diego, CA (US)

(21) Appl. No.: 18/835,663

(22) PCT Filed: Jan. 26, 2023
(86) PCT No.: PCT/US2023/061339

§ 371 Date: Aug. 2, 2024

Related U.S. Application Data
(60) Provisional application No. 63/267,681, filed on Feb. 8, 2022.

(51) Int. Cl. C07K 16/28; A61K 40/11; A61K 40/15; A61K 40/31; A61K 40/42; G01N 33/574; G01N 33/68

(52) U.S. Cl. CPC C07K 16/2866; A61K 40/11; A61K 40/15; A61K 40/31; A61K 40/4217; G01N 33/57492; G01N 33/6869; A61K 2239/13; C07K 2317/24; C07K 2317/31; C07K 2317/565; C07K 2317/732; G01N 2333/7155

(57) ABSTRACT

Anti-IL13Ra2 antibodies and antigen-binding fragments thereof are described. Also described are nucleic acids encoding the antibodies, compositions comprising the antibodies, and methods of producing the antibodies and using the antibodies for treating or preventing diseases such as cancer, inflammatory disease, and/or autoimmune disease.

### CROSS REFERENCE TO RELATED APPLICATIONS

This application claims priority to U.S. Provisional Application No. 63/267,681, filed on Feb. 8, 2022, the disclosure of which is incorporated herein by reference in its entirety.

### FIELD OF THE INVENTION

This invention relates to monoclonal antibodies binding to Interleukin-13 Receptor Subunit Alpha 2 (IL13Ra2), nucleic acids and expression vectors encoding the antibodies, recombinant cells containing the vectors, and compositions comprising the antibodies. Methods of making the antibodies, and methods of using the antibodies to treat diseases including cancer, inflammatory diseases, autoimmune diseases, and/or associated complications are also provided.

### REFERENCE TO SEQUENCE LISTING SUBMITTED ELECTRONICALLY

This application contains a sequence listing, which is submitted electronically. The contents of the electronic sequence listing (065799.36WO1 Sequence Listing.xml; size: 182,964 bytes; and a creation date of Jan. 23, 2023) is herein incorporated by reference in its entirety.

### BACKGROUND OF THE INVENTION

Interleukin-13 receptor alpha 2 (also known as IL13Ra2, IL-13Ra2, IL-13 receptor subunit α2, IL13Rα2, CT19, IL-13R2, IL13BP and CD213A2) is a 42 kD cell surface receptor for IL-13, a proinflammatory cytokine regulating both hematopoietic and non-hematopoietic cells (Marone, G., Granata, F., et al. Frontiers in Pharmacology (2019) 10, 1387). In addition to IL13Ra2, IL-13 also binds to IL13Ra1. IL-13 binds to IL13Ra1 with low affinity and activates the JAK-STAT signaling pathway, which mediates IL-13 mediated cellular responses. Because IL13Ra2 lacks the canonical JAK-STAT signaling function and the binding of IL13 to IL13Ra2 is of high affinity, it was proposed that IL13Ra2 functions as a decoy receptor that negatively regulates IL-13 responses (Rahaman, S. O., Sharma, P., et al. Cancer Research (2002) 63, 1103-1109). More recently, it was reported that IL13Ra2 activates AP-1 mediated transcription in certain cells (Fujisawa, T., Joshi, B. H., et al. International Journal of Cancer (2012) 131, 344-356), suggesting that the function of IL13Ra2 is more complex than previously appreciated.

IL13Ra2 is overexpressed on cancer cells of various types, including glioblastoma multiform (GBM), pancreatic cancers, renal cell carcinoma, head and neck cancers, mesothelioma, melanoma, ovarian cancer, breast cancer, gastric cancer, and colorectal cancer (Suzuki, A., Leland, P., et al. Cytokine (2015) 75, 79-88). IL13Ra2 is expressed at low level on limited tissue cells, making it a useful cancer biomarker and promising therapeutic target. Furthermore, IL13 and IL13Ra2 have been implicated in promoting the growth and invasiveness of epithelial cancers (Fujisawa, T., Joshi, B. H., et al. International Journal of Cancer (2012) 131, 344-356). Taken together, IL13Ra2 is a tumor associated antigen (TAA) and can be an excellent drug target.

### BRIEF SUMMARY OF THE INVENTION

In one general aspect, the invention relates to isolated monoclonal antibodies or antigen-binding fragments thereof that specifically bind IL13Ra2.

Provided are isolated monoclonal antibodies or antigen-binding fragments thereof comprising a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 3, 4, 5, 6, 7 and 8, respectively, or SEQ ID NOs: 9, 10, 11, 12, 13, and 14, respectively;
(2) SEQ ID NOs: 17, 18, 19, 20, 21, and 22, respectively, or SEQ ID NOs: 23, 24, 25, 26, 27, and 28, respectively;
(3) SEQ ID NOs: 31, 32, 33, 34, 35, and 36, respectively or SEQ ID NOs: 37, 38, 39, 40, 41, and 42, respectively;
(4) SEQ ID NOs: 45, 46, 47, 48, 49, and 50, respectively, or SEQ ID NOs: 51, 52, 53, 54, 55, and 56, respectively;
(5) SEQ ID NOs: 59, 60, 61, 62, 63, and 64, respectively, or SEQ ID NOs: 65, 66, 67, 68, 69, and 70, respectively;
(6) SEQ ID NOs: 73, 74, 75, 76, 77, and 78, respectively, or SEQ ID NOs: 79, 80, 81, 82, 83, and 84, respectively;
(7) SEQ ID NOs: 87, 88, 89, 90, 91, and 92, respectively, or SEQ ID NOs: 93, 94, 95, 96, 97, and 98, respectively;
(8) SEQ ID NOs: 101, 102, 103, 104, 105, and 106, respectively, or SEQ ID NOs: 107, 108, 109, 110, 111, and 112, respectively;
(9) SEQ ID NOs: 115, 116, 117, 118, 119, and 120, respectively, or SEQ ID NOs: 121, 122, 123, 124, 125, and 126, respectively;
(10) SEQ ID NOs: 129, 130, 131, 132, 133, and 134, respectively, or SEQ ID NOs: 135, 136, 137, 138, 139, and 140, respectively;
(11) SEQ ID NOs: 143, 144, 145, 146, 147, and 148, respectively, or SEQ ID NOs: 149, 150, 151, 152, 153, and 154, respectively;
(12) SEQ ID NOs: 157, 158, 159, 160, 161, and 162, respectively, or SEQ ID NOs: 163, 164, 165, 166, 167, and 168, respectively; or
(13) SEQ ID NOs: 171, 172, 173, 174, 175, and 176, respectively, or SEQ ID NOs: 177, 178, 179, 180, 181, and 182, respectively;
   wherein the antibody or antigen-binding fragment thereof specifically binds IL13Ra2, preferably human IL13Ra2.

In certain embodiments, the isolated monoclonal antibody or antigen-binding fragment thereof comprises a heavy chain variable region having a polypeptide sequence at least 95%, at least 96%, at least 97%, at least 98%, or at least 99% identical to SEQ ID NO: 1, 15, 29, 43, 57, 71, 85, 99, 113, 127, 141, 155, or 169, or a light chain variable region having a polypeptide sequence at least 95%, at least 96%, at least 97%, at least 98%, or at least 99% identical to SEQ ID NO: 2, 16, 30, 44, 58, 72, 86, 100, 114, 128, 142, 156, or 170.

In certain embodiments, the isolated monoclonal antibody or antigen-binding fragment thereof comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:15, and a light chain variable region having the polypeptide sequence of SEQ ID NO:16;
(3) a heavy chain variable region having the polypeptide sequence of SEQ ID NO:29, and a light chain variable region having the polypeptide sequence of SEQ ID NO:30;
(4) a heavy chain variable region having the polypeptide sequence of SEQ ID NO:43, and a light chain variable region having the polypeptide sequence of SEQ ID NO:44;
(5) a heavy chain variable region having the polypeptide sequence of SEQ ID NO:57, and a light chain variable region having the polypeptide sequence of SEQ ID NO:58;
(6) a heavy chain variable region having the polypeptide sequence of SEQ ID NO:71, and a light chain variable region having the polypeptide sequence of SEQ ID NO:72;
(7) a heavy chain variable region having the polypeptide sequence of SEQ ID NO:85, and a light chain variable region having the polypeptide sequence of SEQ ID NO:86;
(8) a heavy chain variable region having the polypeptide sequence of SEQ ID NO:99, and a light chain variable region having the polypeptide sequence of SEQ ID NO:100;
(9) a heavy chain variable region having the polypeptide sequence of SEQ ID NO:113, and a light chain variable region having the polypeptide sequence of SEQ ID NO:114;
(10) a heavy chain variable region having the polypeptide sequence of SEQ ID NO:127, and a light chain variable region having the polypeptide sequence of SEQ ID NO:128;
(11) a heavy chain variable region having the polypeptide sequence of SEQ ID NO:141, and a light chain variable region having the polypeptide sequence of SEQ ID NO:142;
(12) a heavy chain variable region having the polypeptide sequence of SEQ ID NO:155, and a light chain variable region having the polypeptide sequence of SEQ ID NO:156; or
(13) a heavy chain variable region having the polypeptide sequence of SEQ ID NO:169, and a light chain variable region having the polypeptide sequence of SEQ ID NO:170.

In certain embodiments, the isolated monoclonal antibody or antigen-binding fragment thereof binds to a IL13Ra2 and is capable of inducing effector-mediated tumor cell lysis through antibody-dependent cellular cytotoxicity (ADCC), antibody-dependent cellular phagocytosis (ADCP), and/or complement-dependent cytotoxicity (CDC); and/or mediating the recruitment of conjugated drugs; and/or forming a bispecific antibody with another monoclonal antibody or antigen-binding fragment thereof with cancer-killing effect.

In certain embodiments, the isolated monoclonal antibody or antigen-binding fragment thereof is chimeric.

In certain embodiments, the isolated monoclonal antibody or antigen-binding fragment thereof is human or humanized. In certain embodiments, the humanized monoclonal antibody or antigen-binding fragment thereof comprises a heavy chain variable region having a polypeptide sequence at least 95%, at least 96%, at least 97%, at least 98%, or at least 99% identical to any one of SEQ ID NOs: 183, 184, 185, 186, 191, 192, 193, 197, 198, 199, 203, 204, 207, 208, 211, or 212, or a light chain variable region having a polypeptide sequence at least 95%, at least 96%, at least 97%, at least 98%, or at least 99% identical to any one of SEQ ID NOs: 187, 188, 189, 190, 194, 195, 196, 200, 201, 202, 205, 206, 209, 210, or 213.

In certain embodiments, the humanized monoclonal antibody or antigen-binding fragment thereof comprises:
(1) a heavy chain variable region having the polypeptide sequence of SEQ ID NO:183, and a light chain variable region having the polypeptide sequence of SEQ ID NO:187;
(2) a heavy chain variable region having the polypeptide sequence of SEQ ID NO:183, and a light chain variable region having the polypeptide sequence of SEQ ID NO:188;
(3) a heavy chain variable region having the polypeptide sequence of SEQ ID NO:183, and a light chain variable region having the polypeptide sequence of SEQ ID NO:189;
(4) a heavy chain variable region having the polypeptide sequence of SEQ ID NO:183, and a light chain variable region having the polypeptide sequence of SEQ ID NO:190;
(5) a heavy chain variable region having the polypeptide sequence of SEQ ID NO:184, and a light chain variable region having the polypeptide sequence of SEQ ID NO:187;
(6) a heavy chain variable region having the polypeptide sequence of SEQ ID NO:184, and a light chain variable region having the polypeptide sequence of SEQ ID NO:188;
(7) a heavy chain variable region having the polypeptide sequence of SEQ ID NO:184, and a light chain variable region having the polypeptide sequence of SEQ ID NO:189;
(8) a heavy chain variable region having the polypeptide sequence of SEQ ID NO:184, and a light chain variable region having the polypeptide sequence of SEQ ID NO:190;
(9) a heavy chain variable region having the polypeptide sequence of SEQ ID NO:185, and a light chain variable region having the polypeptide sequence of SEQ ID NO:187;
(10) a heavy chain variable region having the polypeptide sequence of SEQ ID NO:185, and a light chain variable region having the polypeptide sequence of SEQ ID NO:188;
(11) a heavy chain variable region having the polypeptide sequence of SEQ ID NO:185, and a light chain variable region having the polypeptide sequence of SEQ ID NO:189;
(12) a heavy chain variable region having the polypeptide sequence of SEQ ID NO:185, and a light chain variable region having the polypeptide sequence of SEQ ID NO:190;
(13) a heavy chain variable region having the polypeptide sequence of SEQ ID NO:186, and a light chain variable region having the polypeptide sequence of SEQ ID NO:187;
(14) a heavy chain variable region having the polypeptide sequence of SEQ ID NO:186, and a light chain variable region having the polypeptide sequence of SEQ ID NO:188;
(15) a heavy chain variable region having the polypeptide sequence of SEQ ID NO:186, and a light chain variable region having the polypeptide sequence of SEQ ID NO:189;
(16) a heavy chain variable region having the polypeptide sequence of SEQ ID NO:186, and a light chain variable region having the polypeptide sequence of SEQ ID NO:190;
(17) a heavy chain variable region having the polypeptide sequence of SEQ ID NO:191, and a light chain variable region having the polypeptide sequence of SEQ ID NO:194;
(18) a heavy chain variable region having the polypeptide sequence of SEQ ID NO:191, and a light chain variable region having the polypeptide sequence of SEQ ID NO:195;
(19) a heavy chain variable region having the polypeptide sequence of SEQ ID NO:191, and a light chain variable region having the polypeptide sequence of SEQ ID NO:196;
(20) a heavy chain variable region having the polypeptide sequence of SEQ ID NO:192, and a light chain variable region having the polypeptide sequence of SEQ ID NO:194;
(21) a heavy chain variable region having the polypeptide sequence of SEQ ID NO:192, and a light chain variable region having the polypeptide sequence of SEQ ID NO:195;
(22) a heavy chain variable region having the polypeptide sequence of SEQ ID NO:192, and a light chain variable region having the polypeptide sequence of SEQ ID NO:196;
(23) a heavy chain variable region having the polypeptide sequence of SEQ ID NO:193, and a light chain variable region having the polypeptide sequence of SEQ ID NO:194;
(24) a heavy chain variable region having the polypeptide sequence of SEQ ID NO:193, and a light chain variable region having the polypeptide sequence of SEQ ID NO:195;
(25) a heavy chain variable region having the polypeptide sequence of SEQ ID NO:193, and a light chain variable region having the polypeptide sequence of SEQ ID NO:196;
(26) a heavy chain variable region having the polypeptide sequence of SEQ ID NO:197, and a light chain variable region having the polypeptide sequence of SEQ ID NO:200;
(27) a heavy chain variable region having the polypeptide sequence of SEQ ID NO:197, and a light chain variable region having the polypeptide sequence of SEQ ID NO:201;
(28) a heavy chain variable region having the polypeptide sequence of SEQ ID NO:197, and a light chain variable region having the polypeptide sequence of SEQ ID NO:202;
(29) a heavy chain variable region having the polypeptide sequence of SEQ ID NO:198, and a light chain variable region having the polypeptide sequence of SEQ ID NO:200;
(30) a heavy chain variable region having the polypeptide sequence of SEQ ID NO:198, and a light chain variable region having the polypeptide sequence of SEQ ID NO:201;
(31) a heavy chain variable region having the polypeptide sequence of SEQ ID NO:198, and a light chain variable region having the polypeptide sequence of SEQ ID NO:202;
(32) a heavy chain variable region having the polypeptide sequence of SEQ ID NO:199, and a light chain variable region having the polypeptide sequence of SEQ ID NO:200;
(33) a heavy chain variable region having the polypeptide sequence of SEQ ID NO:199, and a light chain variable region having the polypeptide sequence of SEQ ID NO:201;
(34) a heavy chain variable region having the polypeptide sequence of SEQ ID NO:199, and a light chain variable region having the polypeptide sequence of SEQ ID NO:202;
(35) a heavy chain variable region having the polypeptide sequence of SEQ ID NO:203, and a light chain variable region having the polypeptide sequence of SEQ ID NO:205;
(36) a heavy chain variable region having the polypeptide sequence of SEQ ID NO:203, and a light chain variable region having the polypeptide sequence of SEQ ID NO:206;
(37) a heavy chain variable region having the polypeptide sequence of SEQ ID NO:204, and a light chain variable region having the polypeptide sequence of SEQ ID NO:205;
(38) a heavy chain variable region having the polypeptide sequence of SEQ ID NO:204, and a light chain variable region having the polypeptide sequence of SEQ ID NO:206;
(39) a heavy chain variable region having the polypeptide sequence of SEQ ID NO:207, and a light chain variable region having the polypeptide sequence of SEQ ID NO:209;
(40) a heavy chain variable region having the polypeptide sequence of SEQ ID NO:207, and a light chain variable region having the polypeptide sequence of SEQ ID NO:210;
(41) a heavy chain variable region having the polypeptide sequence of SEQ ID NO:208, and a light chain variable region having the polypeptide sequence of SEQ ID NO:209;
(42) a heavy chain variable region having the polypeptide sequence of SEQ ID NO:208, and a light chain variable region having the polypeptide sequence of SEQ ID NO:210;
(43) a heavy chain variable region having the polypeptide sequence of SEQ ID NO:211, and a light chain variable region having the polypeptide sequence of SEQ ID NO:213; or
(44) a heavy chain variable region having the polypeptide sequence of SEQ ID NO:212, and a light chain variable region having the polypeptide sequence of SEQ ID NO:213.

Also provided are isolated bispecific antibodies comprising the monoclonal antibodies or antigen-binding fragments thereof of the invention.

Also provided are isolated nucleic acids encoding the monoclonal antibodies or antigen-binding fragments thereof or bispecific antibodies of the invention.

Also provided are vectors comprising the isolated nucleic acids encoding the monoclonal antibodies or antigen-binding fragments thereof or bispecific antibodies or antigen-binding fragments thereof of the invention.

Also provided are host cells comprising the vectors comprising the isolated nucleic acids encoding the monoclonal antibodies or antigen-binding fragments thereof or bispecific antibodies or antigen-binding fragments thereof of the invention.

In certain embodiments, provided is a pharmaceutical composition comprising an isolated monoclonal antibody or antigen-binding fragment thereof or an isolated bispecific antibody or antigen-binding fragment thereof of the invention and a pharmaceutically acceptable carrier.

Also provided are methods of specifically targeting IL13Ra2, on a cancer cell surface in a subject in need thereof, comprising administering to the subject a pharmaceutical composition of the invention.

Also provided are methods of treating cancer in a subject in need thereof, comprising administering to the subject the pharmaceutical compositions of the invention. The cancer can be any liquid or solid cancer, for example, it can be selected from, but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Also provided are methods of treating an inflammatory disease in a subject in need thereof, comprising administering to the subject a pharmaceutical composition of the invention.

Also provided are methods of treating an autoimmune disease in a subject in need thereof, comprising administering to the subject a pharmaceutical composition of the invention.

Also provided are methods of producing a monoclonal antibody or antigen-binding fragment thereof or bispecific antibody or antigen-binding fragment thereof of the invention, comprising culturing a cell comprising a nucleic acid encoding the monoclonal antibody or antigen-binding fragment thereof or bispecific antibody or antigen-binding fragment thereof under conditions to produce the monoclonal antibody or antigen-binding fragment thereof or bispecific antibody or antigen-binding fragment thereof, and recovering the monoclonal antibody or antigen-binding fragment thereof or bispecific antibody or antigen-binding fragment thereof from the cell or culture.

Also provided are methods of producing a pharmaceutical composition comprising a monoclonal antibody or antigen-binding fragment thereof or bispecific antibody or antigen-binding fragment thereof of the invention, comprising combining the monoclonal antibody or antigen-binding fragment thereof or bispecific antibody or antigen-binding fragment thereof with a pharmaceutically acceptable carrier to obtain the pharmaceutical composition.

Also provided are methods of determining the level of IL13Ra2 in a subject. The methods comprise (a) obtaining a sample from the subject; (b) contacting the sample with an antibody or antigen-binding fragment thereof of the invention; and (c) determining the level of a IL13Ra2 in the subject. In certain embodiments, the sample is a tissue sample. The tissue sample can, for example, be a cancer tissue sample. In certain embodiments, the sample is a blood sample.

In another general aspect, the invention relates to a chimeric antigen receptor (CAR) construct that induces T cell mediated cancer killing, wherein the CAR construct comprises at least one antigen binding domain that specifically binds human IL13Ra2, a hinge region, a transmembrane region, and an intracellular signaling domain.

Provided are isolated polynucleotides comprising a nucleic acid sequence encoding a chimeric antigen receptor (CAR). The CAR can comprise (a) an extracellular domain comprising at least one antigen binding domain that specifically binds IL13Ra2; (b) a hinge region; (c) a transmembrane region; and (d) an intracellular signaling domain.

In certain embodiments, the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 3, 4, 5, 6, 7 and 8, respectively, or SEQ ID NOs: 9, 10, 11, 12, 13, and 14, respectively;
(2) SEQ ID NOs: 17, 18, 19, 20, 21, and 22, respectively, or SEQ ID NOs: 23, 24, 25, 26, 27, and 28, respectively;
(3) SEQ ID NOs: 31, 32, 33, 34, 35, and 36, respectively or SEQ ID NOs: 37, 38, 39, 40, 41, and 42, respectively;
(4) SEQ ID NOs: 45, 46, 47, 48, 49, and 50, respectively, or SEQ ID NOs: 51, 52, 53, 54, 55, and 56, respectively;
(5) SEQ ID NOs: 59, 60, 61, 62, 63, and 64, respectively, or SEQ ID NOs: 65, 66, 67, 68, 69, and 70, respectively;
(6) SEQ ID NOs: 73, 74, 75, 76, 77, and 78, respectively, or SEQ ID NOs: 79, 80, 81, 82, 83, and 84, respectively;
(7) SEQ ID NOs: 87, 88, 89, 90, 91, and 92, respectively, or SEQ ID NOs: 93, 94, 95, 96, 97, and 98, respectively;
(8) SEQ ID NOs: 101, 102, 103, 104, 105, and 106, respectively, or SEQ ID NOs: 107, 108, 109, 110, 111, and 112, respectively;
(9) SEQ ID NOs: 115, 116, 117, 118, 119, and 120, respectively, or SEQ ID NOs: 121, 122, 123, 124, 125, and 126, respectively;
(10) SEQ ID NOs: 129, 130, 131, 132, 133, and 134, respectively, or SEQ ID NOs: 135, 136, 137, 138, 139, and 140, respectively;
(11) SEQ ID NOs: 143, 144, 145, 146, 147, and 148, respectively, or SEQ ID NOs: 149, 150, 151, 152, 153, and 154, respectively;
(12) SEQ ID NOs: 157, 158, 159, 160, 161, and 162, respectively, or SEQ ID NOs: 163, 164, 165, 166, 167, and 168, respectively; or
(13) SEQ ID NOs: 171, 172, 173, 174, 175, and 176, respectively, or SEQ ID NOs: 177, 178, 179, 180, 181, and 182, respectively;
   wherein the antigen binding domain specifically binds IL13Ra2, preferably human IL13Ra2.

In certain embodiments, the antigen binding domain comprises a heavy chain variable region having a polypeptide sequence at least 95%, at least 96%, at least 97%, at least 98%, or at least 99% identical to SEQ ID NO: 1, 15, 29, 43, 57, 71, 85, 99, 113, 127, 141, 155, or 169, or a light chain variable region having a polypeptide sequence at least 95%, at least 96%, at least 97%, at least 98%, or at least 99% identical to SEQ ID NO: 2, 16, 30, 44, 58, 72, 86, 100, 114, 128, 142, 156, or 170.

In certain embodiments, the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:15, and a light chain variable region having the polypeptide sequence of SEQ ID NO:16;
(3) a heavy chain variable region having the polypeptide sequence of SEQ ID NO:29, and a light chain variable region having the polypeptide sequence of SEQ ID NO:30;
(4) a heavy chain variable region having the polypeptide sequence of SEQ ID NO:43, and a light chain variable region having the polypeptide sequence of SEQ ID NO:44;
(5) a heavy chain variable region having the polypeptide sequence of SEQ ID NO:57, and a light chain variable region having the polypeptide sequence of SEQ ID NO:58;
(6) a heavy chain variable region having the polypeptide sequence of SEQ ID NO:71, and a light chain variable region having the polypeptide sequence of SEQ ID NO:72;
(7) a heavy chain variable region having the polypeptide sequence of SEQ ID NO:85, and a light chain variable region having the polypeptide sequence of SEQ ID NO:86;
(8) a heavy chain variable region having the polypeptide sequence of SEQ ID NO:99, and a light chain variable region having the polypeptide sequence of SEQ ID NO:100;
(9) a heavy chain variable region having the polypeptide sequence of SEQ ID NO:113, and a light chain variable region having the polypeptide sequence of SEQ ID NO:114;
(10) a heavy chain variable region having the polypeptide sequence of SEQ ID NO:127, and a light chain variable region having the polypeptide sequence of SEQ ID NO:128;
(11) a heavy chain variable region having the polypeptide sequence of SEQ ID NO:141, and a light chain variable region having the polypeptide sequence of SEQ ID NO:142;
(12) a heavy chain variable region having the polypeptide sequence of SEQ ID NO:155, and a light chain variable region having the polypeptide sequence of SEQ ID NO:156; or
(13) a heavy chain variable region having the polypeptide sequence of SEQ ID NO:169, and a light chain variable region having the polypeptide sequence of SEQ ID NO:170.

In certain embodiments, the antigen binding domain is humanized and comprises a heavy chain variable region having a polypeptide sequence at least 95%, at least 96%, at least 97%, at least 98%, or at least 99% identical to SEQ ID NO: 183, 184, 185, 186, 191, 192, 193, 197, 198, 199, 203, 204, 207, 208, 211, or 212, or a light chain variable region having a polypeptide sequence at least 95%, at least 96%, at least 97%, at least 98%, or at least 99% identical to SEQ ID NO: 187, 188, 189, 190, 194, 195, 196, 200, 201, 202, 205, 206, 209, 210, or 213.

In certain embodiments, the antigen binding domain is humanized and comprises:
(1) a heavy chain variable region having the polypeptide sequence of SEQ ID NO:183, and a light chain variable region having the polypeptide sequence of SEQ ID NO:187;
(2) a heavy chain variable region having the polypeptide sequence of SEQ ID NO:183, and a light chain variable region having the polypeptide sequence of SEQ ID NO:188;
(3) a heavy chain variable region having the polypeptide sequence of SEQ ID NO:183, and a light chain variable region having the polypeptide sequence of SEQ ID NO:189;
(4) a heavy chain variable region having the polypeptide sequence of SEQ ID NO:183, and a light chain variable region having the polypeptide sequence of SEQ ID NO:190;
(5) a heavy chain variable region having the polypeptide sequence of SEQ ID NO:184, and a light chain variable region having the polypeptide sequence of SEQ ID NO:187;
(6) a heavy chain variable region having the polypeptide sequence of SEQ ID NO:184, and a light chain variable region having the polypeptide sequence of SEQ ID NO:188;
(7) a heavy chain variable region having the polypeptide sequence of SEQ ID NO:184, and a light chain variable region having the polypeptide sequence of SEQ ID NO:189;
(8) a heavy chain variable region having the polypeptide sequence of SEQ ID NO:184, and a light chain variable region having the polypeptide sequence of SEQ ID NO:190;
(9) a heavy chain variable region having the polypeptide sequence of SEQ ID NO:185, and a light chain variable region having the polypeptide sequence of SEQ ID NO:187;
(10) a heavy chain variable region having the polypeptide sequence of SEQ ID NO:185, and a light chain variable region having the polypeptide sequence of SEQ ID NO:188;
(11) a heavy chain variable region having the polypeptide sequence of SEQ ID NO:185, and a light chain variable region having the polypeptide sequence of SEQ ID NO:189;
(12) a heavy chain variable region having the polypeptide sequence of SEQ ID NO:185, and a light chain variable region having the polypeptide sequence of SEQ ID NO:190;
(13) a heavy chain variable region having the polypeptide sequence of SEQ ID NO:186, and a light chain variable region having the polypeptide sequence of SEQ ID NO:187;
(14) a heavy chain variable region having the polypeptide sequence of SEQ ID NO:186, and a light chain variable region having the polypeptide sequence of SEQ ID NO:188;
(15) a heavy chain variable region having the polypeptide sequence of SEQ ID NO:186, and a light chain variable region having the polypeptide sequence of SEQ ID NO:189;
(16) a heavy chain variable region having the polypeptide sequence of SEQ ID NO:186, and a light chain variable region having the polypeptide sequence of SEQ ID NO:190;
(17) a heavy chain variable region having the polypeptide sequence of SEQ ID NO:191, and a light chain variable region having the polypeptide sequence of SEQ ID NO:194;
(18) a heavy chain variable region having the polypeptide sequence of SEQ ID NO:191, and a light chain variable region having the polypeptide sequence of SEQ ID NO:195;
(19) a heavy chain variable region having the polypeptide sequence of SEQ ID NO:191, and a light chain variable region having the polypeptide sequence of SEQ ID NO:196;
(20) a heavy chain variable region having the polypeptide sequence of SEQ ID NO:192, and a light chain variable region having the polypeptide sequence of SEQ ID NO:194;
(21) a heavy chain variable region having the polypeptide sequence of SEQ ID NO:192, and a light chain variable region having the polypeptide sequence of SEQ ID NO:195;
(22) a heavy chain variable region having the polypeptide sequence of SEQ ID NO:192, and a light chain variable region having the polypeptide sequence of SEQ ID NO:196;
(23) a heavy chain variable region having the polypeptide sequence of SEQ ID NO:193, and a light chain variable region having the polypeptide sequence of SEQ ID NO:194;
(24) a heavy chain variable region having the polypeptide sequence of SEQ ID NO:193, and a light chain variable region having the polypeptide sequence of SEQ ID NO:195;
(25) a heavy chain variable region having the polypeptide sequence of SEQ ID NO:193, and a light chain variable region having the polypeptide sequence of SEQ ID NO:196;
(26) a heavy chain variable region having the polypeptide sequence of SEQ ID NO:197, and a light chain variable region having the polypeptide sequence of SEQ ID NO:200;
(27) a heavy chain variable region having the polypeptide sequence of SEQ ID NO:197, and a light chain variable region having the polypeptide sequence of SEQ ID NO:201;
(28) a heavy chain variable region having the polypeptide sequence of SEQ ID NO:197, and a light chain variable region having the polypeptide sequence of SEQ ID NO:202;
(29) a heavy chain variable region having the polypeptide sequence of SEQ ID NO:198, and a light chain variable region having the polypeptide sequence of SEQ ID NO:200;
(30) a heavy chain variable region having the polypeptide sequence of SEQ ID NO:198, and a light chain variable region having the polypeptide sequence of SEQ ID NO:201;
(31) a heavy chain variable region having the polypeptide sequence of SEQ ID NO:198, and a light chain variable region having the polypeptide sequence of SEQ ID NO:202;
(32) a heavy chain variable region having the polypeptide sequence of SEQ ID NO:199, and a light chain variable region having the polypeptide sequence of SEQ ID NO:200;
(33) a heavy chain variable region having the polypeptide sequence of SEQ ID NO:199, and a light chain variable region having the polypeptide sequence of SEQ ID NO:201;
(34) a heavy chain variable region having the polypeptide sequence of SEQ ID NO:199, and a light chain variable region having the polypeptide sequence of SEQ ID NO:202;
(35) a heavy chain variable region having the polypeptide sequence of SEQ ID NO:203, and a light chain variable region having the polypeptide sequence of SEQ ID NO:205;
(36) a heavy chain variable region having the polypeptide sequence of SEQ ID NO:203, and a light chain variable region having the polypeptide sequence of SEQ ID NO:206;
(37) a heavy chain variable region having the polypeptide sequence of SEQ ID NO:204, and a light chain variable region having the polypeptide sequence of SEQ ID NO:205;
(38) a heavy chain variable region having the polypeptide sequence of SEQ ID NO:204, and a light chain variable region having the polypeptide sequence of SEQ ID NO:206;
(39) a heavy chain variable region having the polypeptide sequence of SEQ ID NO:207, and a light chain variable region having the polypeptide sequence of SEQ ID NO:209;
(40) a heavy chain variable region having the polypeptide sequence of SEQ ID NO:207, and a light chain variable region having the polypeptide sequence of SEQ ID NO:210;
(41) a heavy chain variable region having the polypeptide sequence of SEQ ID NO:208, and a light chain variable region having the polypeptide sequence of SEQ ID NO:209;
(42) a heavy chain variable region having the polypeptide sequence of SEQ ID NO:208, and a light chain variable region having the polypeptide sequence of SEQ ID NO:210;
(43) a heavy chain variable region having the polypeptide sequence of SEQ ID NO:211, and a light chain variable region having the polypeptide sequence of SEQ ID NO:213; or
(44) a heavy chain variable region having the polypeptide sequence of SEQ ID NO:212, and a light chain variable region having the polypeptide sequence of SEQ ID NO:213.

In certain embodiments, the antigen binding domain is a single chain variable fragment (scFv). In certain embodiments, the antigen binding domain is a humanized single chain variable fragment (scFv).

In certain embodiments, the chimeric antigen receptor (CAR) comprises one or more antigen binding domains.

In certain embodiments, the intracellular signaling domain comprises one or more costimulatory domains and one or more activating domains.

Also provided are chimeric antigen receptors (CARs) encoded by the isolated polynucleotides of the invention.

Also provided are vectors comprising the isolated polynucleotides comprising nucleic acids encoding the CARs of the invention.

Also provided are host cells comprising the vectors of the invention.

In certain embodiments, the host cell is a T cell, preferably a human T cell. In certain embodiments, the host cell is a NK cell, preferably a human NK cell. The T cell or NK cell can, for example, be engineered to express the CAR of the invention to treat diseases such as cancer.

Also provided are methods of making a host cell expressing a chimeric antigen receptor (CAR) of the invention. The methods comprise transducing a T cell or a NK cell with a vector comprising the isolated nucleic acids encoding the CARs of the invention.

Also provided are methods of producing a CAR-T cell or CAR-NK cell of the invention. The methods comprise culturing T cells or NK cells comprising the isolated polynucleotide comprising a nucleic acid encoding a chimeric antigen receptor (CAR) of the invention under conditions to produce the CAR-T cell or CAR-NK cell and recovering the CAR-T cell or CAR-NK cell.

Also provided are methods of generating a population of RNA-engineered cells comprising a chimeric antigen receptor (CAR) of the invention. The methods comprise contacting a cell with the isolated polynucleotide comprising a nucleic acid encoding a chimeric antigen receptor (CAR) of the invention, wherein the isolated polynucleotide is an in vitro transcribed RNA or synthetic RNA.

Also provided are methods of treating cancer in a subject in need thereof, comprising administering to the subject the CAR-T cells and/or CAR-NK cells of the invention. The cancer can be any liquid or solid cancer, for example, it can be selected from, but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In certain embodiments, the methods of treating cancer in a subject in need thereof further comprise administering to the subject in need thereof an agent that increases the efficacy of a cell expressing a CAR molecule.

In certain embodiments, the methods of treating cancer in a subject in need thereof further comprise administering to the subject in need thereof an agent that ameliorates one or more side effects associated with administration of a cell expressing a CAR molecule.

In certain embodiments, the methods of treating cancer in a subject in need thereof further comprise administering to the subject in need thereof an agent that treats the disease associated with IL13Ra2.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1E show the binding of purified chimeric anti-IL13Ra2 mAbs (VH and VL regions of mouse mAb fused to the constant regions of human IgG1 heavy chain and kappa light chain, respectively) to immobilized recombinant IL13Ra2 in an ELISA assay. The antigen was human IL13Ra2 (ACROBiosystems; Newark, DE; CAT #: IL2-H52H5). L3A6-C refers to the chimeric version of L3A6, other antibodies following the same naming rule; Isotype control (IgG1) was included as a negative control antibody.

FIGS. 2A-2B show the binding of anti-IL13Ra2 mAbs to A375 cells (ATCC; Manassas, VA; Cat #: CRL-1619) using FACS analysis. L3A6-C refers to the chimeric version of L3A6, other antibodies following the same naming rule; Isotype control (IgG1), a negative control antibody.

FIGS. 3A-3H show the binding of humanized anti-IL13Ra2 mAbs to immobilized recombinant IL13Ra2 in an ELISA assay. The antigen was human IL13Ra2 (ACROBiosystems; Newark, DE; CAT #: IL2-H52H5). L3A6-H1L1 refers to the humanized mAb constructed with L3A6-H1 heavy chain and L3A6-L1 light chain shown in Table 7; other humanized clones follow the same naming rule. Isotype control (IgG1) was included as a negative control antibody.

FIGS. 4A-4H show the binding of humanized anti-IL13Ra2 mAbs to A375 cells (ATCC; Manassas, VA; Cat #: CRL-1619) using FACS analysis. L3A6-H1L1 refers to the humanized mAb constructed with L3A6-H1 heavy chain and L3A6-L1 light chain shown in Table 7; other humanized clones follow the same naming rule. Isotype control (IgG1), a negative control antibody.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-IL13Ra2 antibodies and polynucleotides that encode them, chimeric antigen receptors (CARs) comprising antigen binding domains specific for IL13Ra2 and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 1981; 2:482, by the homology alignment algorithm of Needleman & Wunsch, J. Mol. Biol. 1970; 48:443, by the search for similarity method of Pearson & Lipman, Proc. Nat'l. Acad. Sci. USA 1988; 85:2444, by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215: 403-410 and Altschul et al., Nucleic Acids Res. 1997; 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Chimeric Antigen Receptor (CAR)

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

In one aspect, the CAR comprises an antigen binding domain, a hinge region, a costimulatory domain, an activating domain and a transmembrane region. In one aspect, the CAR comprises an antigen binding domain, a hinge region, two costimulatory domains, an activating domain and a transmembrane region. In one aspect, the CAR comprises two antigen binding domains, a hinge region, a costimulatory domain, an activating domain and a transmembrane region. In one aspect, the CAR comprises two antigen binding domains, a hinge region, two costimulatory domains, an activating domain and a transmembrane region.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane. It is sometimes referred to as “transmembrane region”.

### Costimulatory Domains

As used herein, chimeric antigen receptors can incorporate costimulatory (signaling) domains to increase their potency. A costimulatory (signaling) domain can be derived from a costimulatory molecule. Costimulatory molecules are cell surface molecules other than antigen receptors or their ligands that are required for an efficient immune response. Costimulatory domains can be derived from costimulatory molecules, which can include, but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CDiia and CD18), CD247, CD276 (B7-H3), LIGHT (tumor necrosis factor superfamily member 14; TNFSFi4), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CDia, CDib, CDic, CDid, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAMI, CRTAM, Ly9 (CD229), CD 160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, or fragments or any combination thereof.

### Activating Domains

As used herein, chimeric antigen receptors can comprise activating domains. Activating domains can include, but are not limited to, CD3. CD3 is an element of the T cell receptor on native T cells and has been shown to be an important intracellular activating element in CARs. In a preferred embodiment, the CD3 is CD3 zeta.

### Hinge Region

As described herein, the chimeric antigen receptor can comprise a hinge region. This is a portion of the extracellular domain, sometimes referred to as a “spacer” region. A variety of hinges can be employed in accordance with the invention, including costimulatory molecules, as discussed above, immunoglobulin (Ig) sequences, or other suitable molecules to achieve the desired special distance from the target cell. In some embodiments, the entire extracellular region comprises a hinge region.

### Transmembrane Region

As used herein, chimeric antigen receptors (CARs) can comprise a transmembrane region/domain. The CAR can be designed to comprise a transmembrane domain that is fused to the extracellular domain of the CAR. It can similarly be fused to the intracellular domain of the CAR. In one embodiment, the transmembrane domain that is naturally associated with one of the domains in a CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention can be derived from (i.e., comprise or engineered from), but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CDIIa and CD18), CD247, CD276 (B7-H3), LIGHT (tumor necrosis factor superfamily member 14; TNFSFi4), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CDia, CDib, CDic, CDid, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAMI, CRTAM, Ly9 (CD229), CD 160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, an immunoglobulin protein, or fragments or any combination thereof.

### Immune Cells

According to particular aspects, the invention provides cells that are immune cells that comprise the isolated polynucleotides or vectors comprising the isolated polynucleotides comprising the nucleotide sequences encoding the CARs are provided herein. The immune cells comprising the isolated polynucleotides and/or vectors of the invention can be referred to as “engineered immune cells.” Preferably, the engineered immune cells are derived from a human (are of human origin prior to being made recombinant).

The engineered immune cells can, for example, be cells of the lymphoid lineage. Non-limiting examples of cells of the lymphoid lineage can include T cells and Natural Killer (NK) cells. T cells express the T cell receptor (TCR), with most cells expressing α and β chains and a smaller population expressing γ and δ chains. T cells useful as engineered immune cells of the invention can be CD4⁺ or CD8⁺ and can include, but are not limited to, T helper cells (CD4⁺), cytotoxic T cells (also referred to as cytotoxic T lymphocytes, CTL; CD8⁺ cells), and memory T cells, including central memory T cells, stem-like memory T cells, and effector memory T cells, natural killer T cells, mucosal associated invariant T cells, and γδ T cells. Other exemplary immune cells include, but are not limited to, macrophages, antigen presenting cells (APCs), or any immune cell that expresses an inhibitor of a cell-mediated immune response, for example, an immune checkpoint inhibitor pathway receptor (e.g., PD-1). Precursor cells of immune cells that can be used according to the invention, include, hematopoietic stem and/or progenitor cells. Hematopoietic stem and/or progenitor cells can be derived from bone marrow, umbilical cord blood, adult peripheral blood after cytokine mobilization, and the like, by methods known in the art. The immune cells are engineered to recombinantly express the CARs of the invention.

Immune cells and precursor cells thereof can be isolated by methods known in the art, including commercially available methods (see, e.g., Rowland Jones et al., Lymphocytes: A Practical Approach, Oxford University Press, NY 1999). Sources for immune cells or precursors thereof include, but are not limited to, peripheral blood, umbilical cord blood, bone marrow, or other sources of hematopoietic cells. Various techniques can be employed to separate the cells to isolated or enrich desired immune cells. For instance, negative selection methods can be used to remove cells that are not the desired immune cells. Additionally, positive selection methods can be used to isolate or enrich for the desired immune cells or precursors thereof, or a combination of positive and negative selection methods can be employed. If a particular type of cell is to be isolated, e.g., a particular T cell, various cell surface markers or combinations of markers (e.g., CD3, CD4, CD8, CD34) can be used to separate the cells.

The immune cells or precursor cells thereof can be autologous or non-autologous to the subject to which they are administered in the methods of treatment of the invention. Autologous cells are isolated from the subject to which the engineered immune cells recombinantly expressing the CAR are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine the appropriate level of compatibility. For both autologous and non-autologous cells, the cells can optionally be cryopreserved until ready for use.

Various methods for isolating immune cells that can be used for recombinant expression of the CARs of the invention have been described previously, and can be used, including, but not limited to, using peripheral donor lymphocytes (Sadelain et al., Nat. Rev. Cancer 2003; 3:35-45; Morgan et al., Science 2006; 314:126-9), using lymphocyte cultures derived from tumor infiltrating lymphocytes (TILs) in tumor biopsies (Panelli et al., J. Immunol. 2000; 164:495-504; Panelli et al., J. Immunol. 2000; 164:4382-92) and using selectively in vitro expanded antigen-specific peripheral blood leukocytes employing artificial antigen-presenting cells (AAPCs) or dendritic cells (Dupont et al., Cancer Res. 2005; 65:5417-427; Papanicolaou et al., Blood 2003; 102:2498-505). In the case of using stem cells, the cells can be isolated by methods well known in the art (see, e.g., Klug et al., Hematopoietic Stem Cell Protocols, Humana Press, NJ 2002; Freshney et al., Culture of Human Stem Cells, John Wiley & Sons 2007).

According to particular embodiments, the method of making the engineered immune cells comprises transfecting or transducing immune effector cells isolated from an individual such that the immune effector cells express one or more CAR(s) according to embodiments of the invention. Methods of preparing immune cells for immunotherapy are described, e.g., in WO2014/130635, WO2013/176916 and WO2013/176915, which are incorporated herein by reference. Individual steps that can be used for preparing engineered immune cells are disclosed, e.g., in WO2014/039523, WO2014/184741, WO2014/191128, WO2014/184744 and WO2014/184143, which are incorporated herein by reference.

In a particular embodiment, the immune effector cells, such as T cells, are genetically modified with CARs of the invention (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694, 6,534,055, 6,905,680, 6,692,964, 5,858,358, 6,887,466, 6,905,681, 7,144,575, 7,067,318, 7,172,869, 7,232,566, 7,175,843, 5,883,223, 6,905,874, 6,797,514, 6,867,041, US2006/121005, which are incorporated herein by reference. T cells can be expanded in vitro or in vivo. Generally, the T cells of the invention can be expanded by contact with a surface having attached thereto an agent that stimulates a CD3/TCR complex-associated signal and a ligand that stimulates a co-stimulatory molecule on the surface of the T cells. As non-limiting examples, T cell populations can be stimulated as described herein, such as by contact with an anti-CD3 antibody, or antigen-binding fragment thereof, or an anti-CD3 antibody immobilized on a surface, or by contact with a protein kinase C activator (e.g., bryostatin) in conjunction with a calcium ionophore, or by activation of the CAR itself. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include, e.g., an appropriate media (e.g., Minimal Essential Media or RPMI Media 1640 or, X-vivo 5 (Lonza)) that can contain factors necessary for proliferation and viability, including serum (e.g., fetal bovine or human serum), cytokines, such as IL-2, IL-7, IL-15, and/or IL-21, insulin, IFN-g, GM-CSF, TGFβ and/or any other additives for the growth of cells known to the skilled artisan. In other embodiments, the T cells can be activated and stimulated to proliferate with feeder cells and appropriate antibodies and cytokines using methods such as those described in U.S. Pat. Nos. 6,040,177, 5,827,642, and WO2012129514, which are incorporated herein by reference.

### Antibodies and Antigen Binding Domains

The invention generally relates to isolated anti-IL13Ra2 antibodies; chimeric antigen receptors (CARs); nucleic acids and expression vectors encoding the antibodies and CARs; recombinant cells containing the vectors; and compositions comprising the antibodies, CARs, and recombinant cells expressing the CARs. The invention also generally relates to methods of making the antibodies and CARs, and methods of using the antibodies and CARs to treat diseases, which can include, but is not limited to, cancer, inflammatory diseases, and autoimmune diseases. The antibodies and antigen binding domains of the CARs of the invention possess one or more desirable functional properties, including, but not limited to, high-affinity binding to IL13Ra2, high specificity to IL13Ra2, the ability to stimulate complement-dependent cytotoxicity (CDC), antibody-dependent cellular phagocytosis (ADCP), and/or antibody-dependent cellular-mediated cytotoxicity (ADCC) against cells expressing IL13Ra2, and the ability to inhibit tumor growth in subjects and animal models when administered alone or in combination with other anti-cancer therapies.

In a general aspect, the invention relates to isolated monoclonal antibodies or antigen-binding fragments thereof that bind IL13Ra2.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

“Complementarity determining regions” (CDR) are antibody regions that bind an antigen. CDRs may be defined using various delineations such as Kabat (Wu et al. J Exp Med 132: 211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196: 901-17, 1987), IMGT (Lefranc et al. Dev Comp Immunol 27: 55-77, 2003) and AbM (Martin and Thornton J Bmol Biol 263: 800-15, 1996). The correspondence between the various delineations and variable region numbering are described (see e.g., Lefranc et al. Dev Comp Immunol 27: 55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g., Kabat; Chothia; Martin; Lefranc et al.).

TABLE A
CDR determinations by different methods.

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

As used herein, the term an “isolated antibody” refers to an antibody which is substantially free of other antibodies having different antigenic specificities (e.g., an isolated antibody that specifically binds to IL13Ra2 is substantially free of antibodies that do not bind to the same IL13Ra2). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” and/or “antigen binding domain” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′). An antigen binding domain is capable of binding to the same antigen to which the parent antibody binds. According to particular embodiments, the antigen binding domain comprises a single-chain antibody molecule (scFv).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” and/or “humanized antigen binding domain” refers to a non-human antibody and/or non-human antigen binding domain that is modified to increase the sequence homology to that of a human antibody and/or a human antigen binding domain, such that the antigen-binding properties of the antibody and/or antigen binding domain are retained, but its antigenicity in the human body is reduced.

As used herein, the term “chimeric antibody” and/or “chimeric antigen binding domain” refers to an antibody and/or antigen binding domain wherein the amino acid sequence of the immunoglobulin molecule is derived from two or more species. The variable region of both the light and heavy chains often corresponds to the variable region of an antibody and/or antigen binding domain derived from one species of mammal (e.g., mouse, rat, rabbit, etc.) having the desired specificity, affinity, and capability, while the constant regions correspond to the sequences of an antibody and/or antigen binding domain derived from another species of mammal (e.g., human) to avoid eliciting an immune response in that species.

As used herein, the term “multi-specific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a multi-specific antibody comprises a third, fourth, or fifth immunoglobulin variable domain. In an embodiment, a multi-specific antibody is a bispecific antibody molecule, a tri-specific antibody molecule, or a tetra-specific antibody molecule.

As used herein, the term “bispecific antibody” refers to a multi-specific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on a IL13Ra2 of this invention and the second epitope is located on PD-1, PD-L1, TIM-3, LAG-3, CTLA-4, EGFR, HER-2, CD19, CD20, CD33, CD3, CD73, CD47, TIP-1, GPC-3, apelin, DLL3, folate receptor alpha, Claudin 18.2 and/or other tumor associated immune suppressors or surface antigens. In an embodiment, the first and the second epitopes are located on the same IL13Ra2 of this invention.

As used herein, an antibody and/or antigen binding domain that “specifically binds to IL13Ra2” refers to an antibody and/or antigen binding domain that binds to the IL13Ra2, preferably the human IL13Ra2, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies and/or antigen binding domains can be determined using methods in the art in view of the present disclosure. For example, the KD of an antibody and/or antigen binding domains can be determined by using surface plasmon resonance, such as by using a biosensor system, e.g., a Biacore® system, or by using bio-layer interferometry technology, such as an Octet RED96 system.

The smaller the value of the KD of an antibody and/or antigen binding domain, the higher affinity that the antibody and/or antigen binding domain binds to a target antigen.

As used herein the term “IC₅₀” refers to the half maximal inhibitory concentration of a monoclonal or bispecific antibody or antigen-binding fragment thereof of the invention. IC₅₀ is a measure of the potency of the monoclonal or bispecific antibody or antigen-binding fragment thereof of the invention for inhibiting the binding of the target antigen (i.e., a receptor or ligand) with its natural ligand or receptor or inhibiting the function of the target antigen in a cell. In certain embodiments, the monoclonal antibody or antigen-binding fragment thereof or the bispecific antibody or antigen-binding fragment thereof has a KD of less than about 10⁻⁷ M, less than about 10⁻⁸ M, less than about 10⁻⁹ M, less than about 10⁻¹⁰ M, less than about 10⁻¹ M, less than about 10⁻¹² M, or less than about 10⁻¹³ M.

As used herein the term “EC₅₀” refers to the half maximal effective concentration of a monoclonal or bispecific antibody or antigen-binding fragment thereof of the invention. EC₅₀ refers to the concentration of a monoclonal or bispecific antibody or antigen-binding fragment thereof for inducing a biological response (i.e., cell death) halfway between the baseline and maximum over a specified exposure time. In certain embodiments, the monoclonal antibody or antigen-binding fragment thereof or the bispecific antibody or antigen-binding fragment thereof has an EC₅₀ of less than about 1 μM, about 1000 nM to about 100 nM, about 100 nM to about 10 nM, about 10 nM to about 1 nM, about 1000 pM to about 500 pM, about 500 pM to about 200 pM, less than about 200 pM, about 200 pM to about 150 pM, about 200 pM to about 100 pM, about 100 pM to about 10 pM, or about 10 pM to about 1 pM.

According to a particular aspect, the invention relates to an isolated monoclonal antibody or antigen-binding fragment thereof or a chimeric antigen receptor (CAR) comprising an antigen binding domain, wherein the monoclonal antibody or antigen-binding fragment thereof or antigen binding domain comprises a heavy chain complementarity determining region 1 (HCDR1), a HCDR2, a HCDR3, a light chain complementarity determining region 1 (LCDR1), a LCDR2, and a LCDR3, having the polypeptide sequences of:
(1) SEQ ID NOs: 3, 4, 5, 6, 7 and 8, respectively, or SEQ ID NOs: 9, 10, 11, 12, 13, and 14, respectively;
(2) SEQ ID NOs: 17, 18, 19, 20, 21, and 22, respectively, or SEQ ID NOs: 23, 24, 25, 26, 27, and 28, respectively;
(3) SEQ ID NOs: 31, 32, 33, 34, 35, and 36, respectively or SEQ ID NOs: 37, 38, 39, 40, 41, and 42, respectively;
(4) SEQ ID NOs: 45, 46, 47, 48, 49, and 50, respectively, or SEQ ID NOs: 51, 52, 53, 54, 55, and 56, respectively;
(5) SEQ ID NOs: 59, 60, 61, 62, 63, and 64, respectively, or SEQ ID NOs: 65, 66, 67, 68, 69, and 70, respectively;
(6) SEQ ID NOs: 73, 74, 75, 76, 77, and 78, respectively, or SEQ ID NOs: 79, 80, 81, 82, 83, and 84, respectively;
(7) SEQ ID NOs: 87, 88, 89, 90, 91, and 92, respectively, or SEQ ID NOs: 93, 94, 95, 96, 97, and 98, respectively;
(8) SEQ ID NOs: 101, 102, 103, 104, 105, and 106, respectively, or SEQ ID NOs: 107, 108, 109, 110, 111, and 112, respectively;
(9) SEQ ID NOs: 115, 116, 117, 118, 119, and 120, respectively, or SEQ ID NOs: 121, 122, 123, 124, 125, and 126, respectively;
(10) SEQ ID NOs: 129, 130, 131, 132, 133, and 134, respectively, or SEQ ID NOs: 135, 136, 137, 138, 139, and 140, respectively;
(11) SEQ ID NOs: 143, 144, 145, 146, 147, and 148, respectively, or SEQ ID NOs: 149, 150, 151, 152, 153, and 154, respectively;
(12) SEQ ID NOs: 157, 158, 159, 160, 161, and 162, respectively, or SEQ ID NOs: 163, 164, 165, 166, 167, and 168, respectively; or
(13) SEQ ID NOs: 171, 172, 173, 174, 175, and 176, respectively, or SEQ ID NOs: 177, 178, 179, 180, 181, and 182, respectively;
   wherein the antibody or antigen-binding fragment thereof or antigen binding domain thereof specifically binds IL13Ra2, preferably human IL13Ra2.

According to another particular aspect, the invention relates to an isolated monoclonal antibody or antigen-binding fragment thereof or a chimeric antigen receptor (CAR) comprising an antigen binding domain, wherein the monoclonal antibody or antigen-binding fragment thereof or antigen binding domain comprises a heavy chain variable region having a polypeptide sequence at least 85%, preferably 90%, more preferably 95% or more, such as 95%, 96%, 97%, 98%, or 99% identical to one of SEQ ID NOs: 1, 15, 29, 43, 57, 71, 85, 99, 113, 127, 141, 155, or 169, or a light chain variable region having a polypeptide sequence at least 85%, preferably 90%, more preferably 95% or more, such as 95%, 96%, 97%, 98%, or 99% identical to one of SEQ ID NOs: 2, 16, 30, 44, 58, 72, 86, 100, 114, 128, 142, 156, or 170.

According to another particular aspect, the invention relates to an isolated monoclonal antibody or antigen-binding fragment thereof or antigen binding domain thereof of the invention, comprising:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:15, and a light chain variable region having the polypeptide sequence of SEQ ID NO:16;
(3) a heavy chain variable region having the polypeptide sequence of SEQ ID NO:29, and a light chain variable region having the polypeptide sequence of SEQ ID NO:30;
(4) a heavy chain variable region having the polypeptide sequence of SEQ ID NO:43, and a light chain variable region having the polypeptide sequence of SEQ ID NO:44;
(5) a heavy chain variable region having the polypeptide sequence of SEQ ID NO:57, and a light chain variable region having the polypeptide sequence of SEQ ID NO:58;
(6) a heavy chain variable region having the polypeptide sequence of SEQ ID NO:71, and a light chain variable region having the polypeptide sequence of SEQ ID NO:72;
(7) a heavy chain variable region having the polypeptide sequence of SEQ ID NO:85, and a light chain variable region having the polypeptide sequence of SEQ ID NO:86;
(8) a heavy chain variable region having the polypeptide sequence of SEQ ID NO:99, and a light chain variable region having the polypeptide sequence of SEQ ID NO:100;
(9) a heavy chain variable region having the polypeptide sequence of SEQ ID NO:113, and a light chain variable region having the polypeptide sequence of SEQ ID NO:114;
(10) a heavy chain variable region having the polypeptide sequence of SEQ ID NO:127, and a light chain variable region having the polypeptide sequence of SEQ ID NO:128;
(11) a heavy chain variable region having the polypeptide sequence of SEQ ID NO:141, and a light chain variable region having the polypeptide sequence of SEQ ID NO:142;
(12) a heavy chain variable region having the polypeptide sequence of SEQ ID NO:155, and a light chain variable region having the polypeptide sequence of SEQ ID NO:156; or
(13) a heavy chain variable region having the polypeptide sequence of SEQ ID NO:169, and a light chain variable region having the polypeptide sequence of SEQ ID NO:170.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 3, 4, 5, 6, 7, and 8, respectively, or SEQ ID NOs: 9, 10, 11, 12, 13, and 14, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:1, and a light chain variable region having a polypeptide sequence at least 85%, preferably 90%, more preferably 95% or more, such as 95%, 96%, 97%, 98%, or 99% identical to SEQ ID NO:2. Preferably, the isolated monoclonal antibody or antigen-binding fragment thereof comprises a heavy chain variable region having the polypeptide sequence of SEQ ID NO:1; and a light chain variable region having the polypeptide sequence of SEQ ID NO:2.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 17, 18, 19, 20, 21, and 22, respectively, or SEQ ID NOs: 23, 24, 25, 26, 27, and 28, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:15, and a light chain variable region having a polypeptide sequence at least 85%, preferably 90%, more preferably 95% or more, such as 95%, 96%, 97%, 98%, or 99% identical to SEQ ID NO:16. Preferably, the isolated monoclonal antibody or antigen-binding fragment thereof comprises a heavy chain variable region having the polypeptide sequence of SEQ ID NO:15; and a light chain variable region having the polypeptide sequence of SEQ ID NO:16.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 31, 32, 33, 34, 35, and 36, respectively, or SEQ ID NOs: 37, 38, 39, 40, 41, and 42, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:29, and a light chain variable region having a polypeptide sequence at least 85%, preferably 90%, more preferably 95% or more, such as 95%, 96%, 97%, 98%, or 99% identical to SEQ ID NO:30. Preferably, the isolated monoclonal antibody or antigen-binding fragment thereof comprises a heavy chain variable region having the polypeptide sequence of SEQ ID NO:29; and a light chain variable region having the polypeptide sequence of SEQ ID NO:30.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 45, 46, 47, 48, 49, and 50, respectively, or SEQ ID NOs: 51, 52, 53, 54, 55, and 56, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 43, and a light chain variable region having a polypeptide sequence at least 85%, preferably 90%, more preferably 95% or more, such as 95%, 96%, 97%, 98%, or 99% identical to SEQ ID NO: 44. Preferably, the isolated monoclonal antibody or antigen-binding fragment thereof comprises a heavy chain variable region having the polypeptide sequence of SEQ ID NO:43; and a light chain variable region having the polypeptide sequence of SEQ ID NO:44.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 59, 60, 61, 62, 63, and 64, respectively, or SEQ ID NOs: 65, 66, 67, 68, 69, and 70, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 57, and a light chain variable region having a polypeptide sequence at least 85%, preferably 90%, more preferably 95% or more, such as 95%, 96%, 97%, 98%, or 99% identical to SEQ ID NO: 58. Preferably, the isolated monoclonal antibody or antigen-binding fragment thereof comprises a heavy chain variable region having the polypeptide sequence of SEQ ID NO:57; and a light chain variable region having the polypeptide sequence of SEQ ID NO:58.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 73, 74, 75, 76, 77, and 78, respectively, or SEQ ID NOs: 79, 80, 81, 82, 83, and 84, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 71, and a light chain variable region having a polypeptide sequence at least 85%, preferably 90%, more preferably 95% or more, such as 95%, 96%, 97%, 98%, or 99% identical to SEQ ID NO: 72. Preferably, the isolated monoclonal antibody or antigen-binding fragment thereof comprises a heavy chain variable region having the polypeptide sequence of SEQ ID NO:71; and a light chain variable region having the polypeptide sequence of SEQ ID NO:72.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 87, 88, 89, 90, 91, and 92, respectively, or SEQ ID NOs: 93, 94, 95, 96, 97, and 98, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 85, and a light chain variable region having a polypeptide sequence at least 85%, preferably 90%, more preferably 95% or more, such as 95%, 96%, 97%, 98%, or 99% identical to SEQ ID NO: 86. Preferably, the isolated monoclonal antibody or antigen-binding fragment thereof comprises a heavy chain variable region having the polypeptide sequence of SEQ ID NO:85; and a light chain variable region having the polypeptide sequence of SEQ ID NO:86.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 101, 102, 103, 104, 105, and 106, respectively, or SEQ ID NOs: 107, 108, 109, 110, 111, and 112, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 99, and a light chain variable region having a polypeptide sequence at least 85%, preferably 90%, more preferably 95% or more, such as 95%, 96%, 97%, 98%, or 99% identical to SEQ ID NO: 100. Preferably, the isolated monoclonal antibody or antigen-binding fragment thereof comprises a heavy chain variable region having the polypeptide sequence of SEQ ID NO:99; and a light chain variable region having the polypeptide sequence of SEQ ID NO:100.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 115, 116, 117, 118, 119, and 120, respectively, or SEQ ID NOs: 121, 122, 123, 124, 125, and 126, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 113, and a light chain variable region having a polypeptide sequence at least 85%, preferably 90%, more preferably 95% or more, such as 95%, 96%, 97%, 98%, or 99% identical to SEQ ID NO: 114. Preferably, the isolated monoclonal antibody or antigen-binding fragment thereof comprises a heavy chain variable region having the polypeptide sequence of SEQ ID NO:113; and a light chain variable region having the polypeptide sequence of SEQ ID NO:114.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 129, 130, 131, 132, 133, and 134, respectively, or SEQ ID NOs: 135, 136, 137, 138, 139, and 140, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 127, and a light chain variable region having a polypeptide sequence at least 85%, preferably 90%, more preferably 95% or more, such as 95%, 96%, 97%, 98%, or 99% identical to SEQ ID NO: 128. Preferably, the isolated monoclonal antibody or antigen-binding fragment thereof comprises a heavy chain variable region having the polypeptide sequence of SEQ ID NO:127; and a light chain variable region having the polypeptide sequence of SEQ ID NO:128.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 143, 144, 145, 146, 147, and 148, respectively, or SEQ ID NOs: 149, 150, 151, 152, 153, and 154, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 141, and a light chain variable region having a polypeptide sequence at least 85%, preferably 90%, more preferably 95% or more, such as 95%, 96%, 97%, 98%, or 99% identical to SEQ ID NO: 142. Preferably, the isolated monoclonal antibody or antigen-binding fragment thereof comprises a heavy chain variable region having the polypeptide sequence of SEQ ID NO:141; and a light chain variable region having the polypeptide sequence of SEQ ID NO:142.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 157, 158, 159, 160, 161, and 162, respectively, or SEQ ID NOs: 163, 164, 165, 166, 167, and 168, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 155, and a light chain variable region having a polypeptide sequence at least 85%, preferably 90%, more preferably 95% or more, such as 95%, 96%, 97%, 98%, or 99% identical to SEQ ID NO: 156. Preferably, the isolated monoclonal antibody or antigen-binding fragment thereof comprises a heavy chain variable region having the polypeptide sequence of SEQ ID NO:155; and a light chain variable region having the polypeptide sequence of SEQ ID NO:156.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 171, 172, 173, 174, 175, and 176, respectively, or SEQ ID NOs: 177, 178, 179, 180, 181, and 182, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 169, and a light chain variable region having a polypeptide sequence at least 85%, preferably 90%, more preferably 95% or more, such as 95%, 96%, 97%, 98%, or 99% identical to SEQ ID NO: 170. Preferably, the isolated monoclonal antibody or antigen-binding fragment thereof comprises a heavy chain variable region having the polypeptide sequence of SEQ ID NO:169; and a light chain variable region having the polypeptide sequence of SEQ ID NO:170.

According to another particular aspect, the invention relates to an isolated monoclonal antibody or antigen-binding fragment thereof of the invention, wherein the antibody or antigen-binding fragment thereof is chimeric.

According to another particular aspect, the invention relates to an isolated monoclonal antibody or antigen-binding fragment thereof of the invention, wherein the antibody or antigen-binding fragment thereof is human or humanized.

According to another particular aspect, the humanized monoclonal antibody or antigen-binding fragment thereof comprises a heavy chain variable region having a polypeptide sequence at least 95%, at least 96%, at least 97%, at least 98%, or at least 99% identical to any one of SEQ ID NOs: 183, 184, 185, 186, 191, 192, 193, 197, 198, 199, 203, 204, 207, 208, 211, or 212, or a light chain variable region having a polypeptide sequence at least 95%, at least 96%, at least 97%, at least 98%, or at least 99% identical to any one of SEQ ID NOs: 187, 188, 189, 190, 194, 195, 196, 200, 201, 202, 205, 206, 209, 210, or 213.

According to another particular aspect, the humanized monoclonal antibody or antigen-binding fragment thereof comprises:
(1) a heavy chain variable region having the polypeptide sequence of SEQ ID NO:183, and a light chain variable region having the polypeptide sequence of SEQ ID NO:187;
(2) a heavy chain variable region having the polypeptide sequence of SEQ ID NO:183, and a light chain variable region having the polypeptide sequence of SEQ ID NO:188;
(3) a heavy chain variable region having the polypeptide sequence of SEQ ID NO:183, and a light chain variable region having the polypeptide sequence of SEQ ID NO:189;
(4) a heavy chain variable region having the polypeptide sequence of SEQ ID NO:183, and a light chain variable region having the polypeptide sequence of SEQ ID NO:190;
(5) a heavy chain variable region having the polypeptide sequence of SEQ ID NO:184, and a light chain variable region having the polypeptide sequence of SEQ ID NO:187;
(6) a heavy chain variable region having the polypeptide sequence of SEQ ID NO:184, and a light chain variable region having the polypeptide sequence of SEQ ID NO:188;
(7) a heavy chain variable region having the polypeptide sequence of SEQ ID NO:184, and a light chain variable region having the polypeptide sequence of SEQ ID NO:189;
(8) a heavy chain variable region having the polypeptide sequence of SEQ ID NO:184, and a light chain variable region having the polypeptide sequence of SEQ ID NO:190;
(9) a heavy chain variable region having the polypeptide sequence of SEQ ID NO:185, and a light chain variable region having the polypeptide sequence of SEQ ID NO:187;
(10) a heavy chain variable region having the polypeptide sequence of SEQ ID NO:185, and a light chain variable region having the polypeptide sequence of SEQ ID NO:188;
(11) a heavy chain variable region having the polypeptide sequence of SEQ ID NO:185, and a light chain variable region having the polypeptide sequence of SEQ ID NO:189;
(12) a heavy chain variable region having the polypeptide sequence of SEQ ID NO:185, and a light chain variable region having the polypeptide sequence of SEQ ID NO:190;
(13) a heavy chain variable region having the polypeptide sequence of SEQ ID NO:186, and a light chain variable region having the polypeptide sequence of SEQ ID NO:187;
(14) a heavy chain variable region having the polypeptide sequence of SEQ ID NO:186, and a light chain variable region having the polypeptide sequence of SEQ ID NO:188;
(15) a heavy chain variable region having the polypeptide sequence of SEQ ID NO:186, and a light chain variable region having the polypeptide sequence of SEQ ID NO:189;
(16) a heavy chain variable region having the polypeptide sequence of SEQ ID NO:186, and a light chain variable region having the polypeptide sequence of SEQ ID NO:190;
(17) a heavy chain variable region having the polypeptide sequence of SEQ ID NO:191, and a light chain variable region having the polypeptide sequence of SEQ ID NO:194;
(18) a heavy chain variable region having the polypeptide sequence of SEQ ID NO:191, and a light chain variable region having the polypeptide sequence of SEQ ID NO:195;
(19) a heavy chain variable region having the polypeptide sequence of SEQ ID NO:191, and a light chain variable region having the polypeptide sequence of SEQ ID NO:196;
(20) a heavy chain variable region having the polypeptide sequence of SEQ ID NO:192, and a light chain variable region having the polypeptide sequence of SEQ ID NO:194;
(21) a heavy chain variable region having the polypeptide sequence of SEQ ID NO:192, and a light chain variable region having the polypeptide sequence of SEQ ID NO:195;
(22) a heavy chain variable region having the polypeptide sequence of SEQ ID NO:192, and a light chain variable region having the polypeptide sequence of SEQ ID NO:196;
(23) a heavy chain variable region having the polypeptide sequence of SEQ ID NO:193, and a light chain variable region having the polypeptide sequence of SEQ ID NO:194;
(24) a heavy chain variable region having the polypeptide sequence of SEQ ID NO:193, and a light chain variable region having the polypeptide sequence of SEQ ID NO:195;
(25) a heavy chain variable region having the polypeptide sequence of SEQ ID NO:193, and a light chain variable region having the polypeptide sequence of SEQ ID NO:196;
(26) a heavy chain variable region having the polypeptide sequence of SEQ ID NO:197, and a light chain variable region having the polypeptide sequence of SEQ ID NO:200;
(27) a heavy chain variable region having the polypeptide sequence of SEQ ID NO:197, and a light chain variable region having the polypeptide sequence of SEQ ID NO:201;
(28) a heavy chain variable region having the polypeptide sequence of SEQ ID NO:197, and a light chain variable region having the polypeptide sequence of SEQ ID NO:202;
(29) a heavy chain variable region having the polypeptide sequence of SEQ ID NO:198, and a light chain variable region having the polypeptide sequence of SEQ ID NO:200;
(30) a heavy chain variable region having the polypeptide sequence of SEQ ID NO:198, and a light chain variable region having the polypeptide sequence of SEQ ID NO:201;
(31) a heavy chain variable region having the polypeptide sequence of SEQ ID NO:198, and a light chain variable region having the polypeptide sequence of SEQ ID NO:202;
(32) a heavy chain variable region having the polypeptide sequence of SEQ ID NO:199, and a light chain variable region having the polypeptide sequence of SEQ ID NO:200;
(33) a heavy chain variable region having the polypeptide sequence of SEQ ID NO:199, and a light chain variable region having the polypeptide sequence of SEQ ID NO:201;
(34) a heavy chain variable region having the polypeptide sequence of SEQ ID NO:199, and a light chain variable region having the polypeptide sequence of SEQ ID NO:202;
(35) a heavy chain variable region having the polypeptide sequence of SEQ ID NO:203, and a light chain variable region having the polypeptide sequence of SEQ ID NO:205;
(36) a heavy chain variable region having the polypeptide sequence of SEQ ID NO:203, and a light chain variable region having the polypeptide sequence of SEQ ID NO:206;
(37) a heavy chain variable region having the polypeptide sequence of SEQ ID NO:204, and a light chain variable region having the polypeptide sequence of SEQ ID NO:205;
(38) a heavy chain variable region having the polypeptide sequence of SEQ ID NO:204, and a light chain variable region having the polypeptide sequence of SEQ ID NO:206;
(39) a heavy chain variable region having the polypeptide sequence of SEQ ID NO:207, and a light chain variable region having the polypeptide sequence of SEQ ID NO:209;
(40) a heavy chain variable region having the polypeptide sequence of SEQ ID NO:207, and a light chain variable region having the polypeptide sequence of SEQ ID NO:210;
(41) a heavy chain variable region having the polypeptide sequence of SEQ ID NO:208, and a light chain variable region having the polypeptide sequence of SEQ ID NO:209;
(42) a heavy chain variable region having the polypeptide sequence of SEQ ID NO:208, and a light chain variable region having the polypeptide sequence of SEQ ID NO:210;
(43) a heavy chain variable region having the polypeptide sequence of SEQ ID NO:211, and a light chain variable region having the polypeptide sequence of SEQ ID NO:213; or
(44) a heavy chain variable region having the polypeptide sequence of SEQ ID NO:212, and a light chain variable region having the polypeptide sequence of SEQ ID NO:213.

According to another particular aspect, the antigen binding domain is a single chain variable fragment (scFv) that specifically binds IL13Ra2, preferably human IL13Ra2.

In certain embodiments, the encoded antigen binding domain is a humanized single chain variable fragment (scFv) that specifically binds IL13Ra2, preferably human IL13Ra2. In certain embodiments, the antigen binding domain is a humanized single chain variable fragment (scFv) that specifically binds IL13Ra2, preferably human IL13Ra2.

In certain embodiments, the encoded antigen binding domain is a humanized single chain variable fragment (scFv) that specifically binds IL13Ra2, preferably human IL13Ra2. In certain embodiments, the antigen binding domain is a humanized single chain variable fragment (scFv) that specifically binds IL13Ra2, preferably human IL13Ra2.

According to another particular aspect, the chimeric antigen receptor comprises one or more antigen binding domains.

According to another particular aspect, the intracellular signaling domain comprises one or more costimulatory domains and one or more activating domains.

In another general aspect, the invention relates to an isolated nucleic acid encoding a monoclonal antibody or antigen-binding fragment thereof and/or a bispecific antibody or antigen-binding fragment thereof of the invention. In another general aspect, the invention relates to an isolated polynucleotide comprising a nucleic acid encoding a chimeric antigen receptor (CAR), wherein the CAR comprises an antigen binding domain thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies or antigen-binding fragments thereof of the invention can be altered without changing the amino acid sequences of the proteins.

In another general aspect, the invention relates to a vector comprising an isolated nucleic acid encoding a monoclonal antibody or antigen-binding fragment thereof, a bispecific antibody or antigen-binding fragment thereof, and/or a CAR of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In another general aspect, the invention relates to a host cell comprising an isolated nucleic acid encoding a monoclonal antibody or antigen-binding fragment thereof and/or a bispecific antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a monoclonal antibody or antigen-binding fragment thereof and/or a bispecific antibody or antigen-binding fragment thereof of the invention, comprising culturing a cell comprising a nucleic acid encoding the monoclonal antibody or antigen-binding fragment thereof or bispecific antibody or antigen-binding fragment thereof under conditions to produce a monoclonal antibody or antigen-binding fragment thereof or bispecific antibody or antigen-binding fragment thereof of the invention, and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

In another general aspect, the invention relates to a cell transduced with the vector comprising the isolated nucleic acids encoding the CARs of the invention. The term “transduced” or “transduction” refers to a process by which exogenous nucleic acid is transferred or introduced into the host cell. A “transduced” cell is one which has been transduced with exogenous nucleic acid. The cell includes the primary subject cell and its progeny. In certain embodiments, the cell is a CAR-T cell, preferably a human CAR-T cell, wherein the T cell is engineered to express the CAR of the invention to treat diseases such as cancer. In certain embodiments, the cell is a CAR-NK cell, preferably a human CAR-NK cell, wherein the NK cell engineered to express the CAR of the invention is used to treat diseases such as cancer.

In another general aspect, the invention relates to a method of making a CAR-T cell by transducing a T cell with a vector comprising the isolated nucleic acids encoding the CARs of the invention.

In another general aspect, the invention relates to a method of producing the CAR-T cell thereof of the invention, comprising culturing T cells comprising a nucleic acid encoding a chimeric antigen receptor (CAR) of the invention under conditions to produce the CAR-T cell, and recovering the CAR-T cell.

In another general aspect, the invention relates to a method of making a CAR-NK cell by transducing a NK cell with a vector comprising the isolated nucleic acids encoding the CARs of the invention.

In another general aspect, the invention relates to a method of producing a CAR-NK cell of the invention, comprising culturing NK cells comprising nucleic acids encoding the chimeric antigen receptor (CAR) thereof under conditions to produce the CAR-NK cell, and recovering the CAR-NK cell.

In another general aspect, the invention relates to a method of generating a population of RNA-engineered cells comprising a chimeric antigen receptor (CAR) of the invention. The methods comprise contacting a population of cells with isolated polynucleotides comprising a nucleic acid encoding a CAR of the invention, wherein the isolated polynucleotides are in vitro transcribed RNA or synthetic RNA.

### Pharmaceutical Compositions

In another general aspect, the invention relates to a pharmaceutical composition, comprising an isolated monoclonal antibody or antigen-binding fragment thereof, a bispecific antibody or antigen-binding fragment thereof, an isolated polynucleotide, an isolated polypeptide, a host cell, and/or an engineered immune cell of the invention and a pharmaceutically acceptable carrier.

The term “pharmaceutical composition” as used herein means a product comprising an isolated polynucleotide of the invention, an isolated polypeptide of the invention, a host cell of the invention, an engineered immune cell of the invention, an anti-IL13Ra2 monoclonal antibody or antigen-binding fragment thereof, and/or a bispecific antibody of the invention together with a pharmaceutically acceptable carrier. Polynucleotides, polypeptides, host cells, engineered immune cells of the invention, an anti-IL13Ra2 monoclonal antibody or antigen-binding fragment thereof, and/or a bispecific antibody of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

The formulation of pharmaceutically active ingredients with pharmaceutically acceptable carriers is known in the art, e.g., Remington: The Science and Practice of Pharmacy (e.g., 21st edition (2005), and any later editions). Non-limiting examples of additional ingredients include buffers, diluents, solvents, tonicity regulating agents, preservatives, stabilizers, and chelating agents. One or more pharmaceutically acceptable carrier(s) can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms may be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g., PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivatives thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a monoclonal antibody or antigen-binding fragment thereof and/or a bispecific antibody or antigen-binding fragment thereof of the invention, comprising combining a monoclonal antibody or antigen-binding fragment thereof and/or a bispecific antibody or antigen-binding fragment thereof with a pharmaceutically acceptable carrier to obtain the pharmaceutical composition.

### Methods of Use

In another general aspect, the invention relates to a method of targeting IL13Ra2 on a cancer cell surface in a subject to achieve cell killing, the method comprising administering to the subject an isolated monoclonal antibody or antigen binding fragment thereof and/or bispecific antibody or antigen-binding fragment thereof that specifically binds the IL13Ra2 or a pharmaceutical composition comprising the isolated monoclonal antibody or antigen binding fragment thereof and/or bispecific antibody or antigen-binding fragment thereof of the invention. Binding of the IL13Ra2 monoclonal or bispecific antibody or antigen-binding fragment to the IL13Ra2 can mediate complement-dependent cytotoxicity (CDC), antibody-dependent cellular phagocytosis (ADCP), and/or antibody-dependent cellular cytotoxicity (ADCC) or other effects that result in the death of the targeted cancer cell. The monoclonal or bispecific antibody or antigen binding fragment thereof can, for example, serve to recruit conjugated drugs, and/or can form a bispecific antibody with another monoclonal antibody to mediate the death of the targeted cancer cell.

The functional activity of antibodies and antigen-binding fragments thereof that bind IL13Ra2 can be characterized by methods known in the art and as described herein. Methods for characterizing antibodies and antigen-binding fragments thereof that bind a IL13Ra2 include, but are not limited to, affinity and specificity assays including Biacore, ELISA, and OctetRed analysis, and detection of the binding of antibodies and antigen-binding fragments to the IL13Ra2 on cells (either cells transfected with the IL13Ra2 or cells that naturally express the IL13Ra2) by FACS. According to particular embodiments, the methods for characterizing antibodies and antigen-binding fragments thereof that bind IL13Ra2 include those described below.

In another general aspect, the invention relates to a method of treating a cancer in a subject in need thereof, comprising administering to the subject an isolated monoclonal antibody or antigen-binding fragment thereof and/or bispecific antibody or antigen-binding fragment thereof that specifically binds a IL13Ra2 or a pharmaceutical composition of the invention. The cancer can, for example, be selected from but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In another general aspect, the invention relates to a method of treating an inflammatory disease in a subject in need thereof, comprising administering to the subject an isolated monoclonal antibody or antigen-binding fragment thereof and/or bispecific antibody or antigen-binding fragment thereof that specifically binds IL13Ra2 or a pharmaceutical composition of the invention.

In another general aspect, the invention relates to a method of treating an autoimmune disease in a subject in need thereof, comprising administering to the subject an isolated monoclonal antibody or antigen-binding fragment thereof and/or bispecific antibody or antigen-binding fragment thereof that specifically binds IL13Ra2 or a pharmaceutical composition of the invention.

In another general aspect, the invention relates to a method of treating a cancer in a subject in need thereof, comprising administering to the subject the CAR-T cells and/or CAR-NK cells of the invention. The cancer can, for example, be selected from but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the CAR-T cell or CAR-NK cells comprise a therapeutically effective amount of the expressed CARs of the invention and the pharmaceutical composition comprises a therapeutically effective amount of an anti-IL13Ra2 antibody or antigen-binding fragment thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to CARs, a therapeutically effective amount means an amount of the CAR molecule expressed in the transduced T cell or NK cell that modulates an immune response in a subject in need thereof. Also, as used herein with reference to CARs, a therapeutically effective amount means an amount of the CAR molecule expressed in the transduced T cell or NK cell that results in treatment of a disease, disorder, or condition; prevents or slows the progression of the disease, disorder, or condition; or reduces or completely alleviates symptoms associated with the disease, disorder, or condition.

As used herein with reference to CAR-T cell or CAR-NK cell, a therapeutically effective amount means an amount of the CAR-T cells or CAR-NK cells that modulates an immune response in a subject in need thereof. Also, as used herein with reference to CAR-T cell or CAR-NK cell, a therapeutically effective amount means an amount of the CAR-T cells or CAR-NK cells that results in treatment of a disease, disorder, or condition; prevents or slows the progression of the disease, disorder, or condition; or reduces or completely alleviates symptoms associated with the disease, disorder, or condition.

As used herein with reference to anti-IL13Ra2 antibodies or antigen-binding fragments thereof, a therapeutically effective amount means an amount of the anti-IL13Ra2 antibody or antigen-binding fragment thereof that modulates an immune response in a subject in need thereof. Also, as used herein with reference to anti-IL13Ra2 antibodies or antigen-binding fragments thereof, a therapeutically effective amount means an amount of the anti-IL13Ra2 antibody or antigen-binding fragment thereof that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to other particular embodiments, the disease, disorder or condition to be treated is an inflammatory disease. According to other particular embodiments, the disease, disorder, or condition to be treated is an autoimmune disease.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the invention can be administered in any convenient manner known to those skilled in the art. For example, the cells of the invention can be administered to the subject by aerosol inhalation, injection, ingestion, transfusion, implantation, and/or transplantation. The compositions comprising the cells of the invention can be administered transarterially, subcutaneously, intradermally, intratumorally, intranodally, intramedullary, intramuscularly, intrapleurally, by intravenous (i.v.) injection, or intraperitoneally. In certain embodiments, the cells of the invention can be administered with or without lymphodepletion of the subject.

The pharmaceutical compositions comprising cells of the invention expressing CARs of the invention can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the invention.

The cells of the invention can be administered in any physiologically acceptable vehicle. A cell population comprising cells of the invention can comprise a purified population of cells. Those skilled in the art can readily determine the cells in a cell population using various well-known methods. The ranges in purity in cell populations comprising genetically modified cells of the invention can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the invention are generally administered as a dose based on cells per kilogram (cells/kg) of body weight of the subject to which the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the invention are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer and/or an inflammatory or autoimmune disease, disorder or condition, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer and/or an inflammatory disease, disorder or condition. For cancer therapy, the provided compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-DLL-3 mAb, an anti-apelin mAb, an anti-FOLR1 mAb, an anti-CTLA-4 mAb, an anti-PD-L1 mAb, an anti-PD-1 mAb, an anti-Claudin 18.2 mAb, other immuno-oncology drugs, an antiangiogenic agent, a radiation therapy, an antibody-drug conjugate (ADC), a targeted therapy, or other anticancer drugs. Antibodies against IL13Ra2 can be used to construct bispecific antibodies with partner mAbs against PD-1, PD-L1, LAG3, TIM-3, CTLA-4, EGFR, HER-2, CD19, CD20, CD33, CD73, CD47, CD3, apelin, DLL-3, TIP-1, GPC3, Claudin 18.2, folate receptor alpha (FOLR1), and/or any other TAA to treat cancers/tumors that express both antigens. Two antibodies that recognize two different epitopes on IL13Ra2 can also be used to construct a bispecific antibody to treat cancers/tumors that express the IL13Ra2.

According to particular embodiments, the methods of treating cancer in a subject in need thereof comprise administering to the subject the CAR-T cells and/or CAR-NK cells of the invention in combination with an agent that increases the efficacy of a cell expressing a CAR molecule. Such agents include, but are not limited to, an antibody fragment that binds to CD73, CD39, PD1, PD-L1, PD-L2, CTLA4, TIM3 or LAG3, or an adenosine A2a receptor antagonist.

According to particular embodiments, the methods of treating cancer in a subject in need thereof comprise administering to the subject the CAR-T cells and/or CAR-NK cells of the invention in combination with an agent that ameliorates one or more side effects associated with administration of a cell expressing a CAR molecule. Such agents include, but are not limited to, a steroid, an inhibitor of TNFα, or an inhibitor of IL-6.

According to particular embodiments, the methods of treating cancer in a subject in need thereof comprise administering to the subject the CAR-T cells and/or CAR-NK cells of the invention in combination with an agent that treats the disease associated with IL13Ra2. Such agents include, but are not limited to, an anti-IL13Ra2 monoclonal antibody or bispecific antibody.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

In another general aspect, the invention relates to a method of determining a level of IL13Ra2 in a subject. The methods comprise (a) obtaining a sample from the subject; (b) contacting the sample with a monoclonal antibody or antigen-binding fragment thereof of the invention; and (c) determining a level of the IL13Ra2 in the subject.

As used herein, “sample” refers to a biological sample isolated from a subject and can include, but is not limited to, whole blood, serum, plasma, blood cells, endothelial cells, tissue biopsies (e.g., a cancer tissue), lymphatic fluid, ascites fluid, interstitial fluid, bone marrow, cerebrospinal fluid, saliva, mucous, sputum, sweat, urine, or any other secretion, excretion, or other bodily fluids. A “blood sample” refers to whole blood or any fraction thereof, including blood cells, serum, and plasma.

In certain embodiments, the level of IL13Ra2 in the subject can be determined utilizing assays selected from, but not limited to, a Western blot assay, immunohistochemistry (IHC) and an ELISA assay. Relative protein levels can be determined by utilizing Western blot analysis and IHC, and absolute protein levels can be determined by utilizing an ELISA assay. When determining the relative levels of a IL13Ra2, the levels of the IL13Ra2 can be determined between at least two samples, e.g., between samples from the same subject at different time points, between samples from different tissues in the same subject, and/or between samples from different subjects. Alternatively, when determining absolute levels of the IL13Ra2, such as by an ELISA assay, the absolute level of the IL13Ra2 in the sample can be determined by creating a standard for the ELISA assay prior to testing the sample. A person skilled in the art would understand which analytical techniques to utilize to determine the level of IL13Ra2 in a sample from the subject utilizing the antibodies or antigen-binding fragments thereof of the invention.

Utilizing methods of determining a level of a IL13Ra2 in a sample from a subject can lead to the diagnosis of abnormal (elevated, reduced, or insufficient) IL13Ra2 levels in a disease and making appropriate therapeutic decisions. Such a disease can be selected from, but not limited to, a cancer, an inflammatory disease, and an autoimmune disease. Additionally, by monitoring the levels of IL13Ra2 in a subject, the risk of developing a disease as indicated above can be determined based on the knowledge of the level of the IL13Ra2 in a particular disease and/or during the progression of the particular disease.

### EMBODIMENTS

The invention provides also the following non-limiting embodiments.

Embodiment 1 is an isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 3, 4, 5, 6, 7, and 8, respectively; or SEQ ID NOs: 9, 10, 11, 12, 13, and 14, respectively;
(2) SEQ ID NOs: 17, 18, 19, 20, 21, and 22, respectively, or SEQ ID NOs: 23, 24, 25, 26, 27, and 28, respectively;
(3) SEQ ID NOs: 31, 32, 33, 34, 35, and 36, respectively or SEQ ID NOs: 37, 38, 39, 40, 41, and 42, respectively;
(4) SEQ ID NOs: 45, 46, 47, 48, 49, and 50, respectively, or SEQ ID NOs: 51, 52, 53, 54, 55, and 56, respectively;
(5) SEQ ID NOs: 59, 60, 61, 62, 63, and 64, respectively, or SEQ ID NOs: 65, 66, 67, 68, 69, and 70, respectively;
(6) SEQ ID NOs: 73, 74, 75, 76, 77, and 78, respectively, or SEQ ID NOs: 79, 80, 81, 82, 83, and 84, respectively;
(7) SEQ ID NOs: 87, 88, 89, 90, 91, and 92, respectively, or SEQ ID NOs: 93, 94, 95, 96, 97, and 98, respectively;
(8) SEQ ID NOs: 101, 102, 103, 104, 105, and 106, respectively, or SEQ ID NOs: 107, 108, 109, 110, 111, and 112, respectively;
(9) SEQ ID NOs: 115, 116, 117, 118, 119, and 120, respectively, or SEQ ID NOs: 121, 122, 123, 124, 125, and 126, respectively;
(10) SEQ ID NOs: 129, 130, 131, 132, 133, and 134, respectively, or SEQ ID NOs: 135, 136, 137, 138, 139, and 140, respectively;
(11) SEQ ID NOs: 143, 144, 145, 146, 147, and 148, respectively, or SEQ ID NOs: 149, 150, 151, 152, 153, and 154, respectively;
(12) SEQ ID NOs: 157, 158, 159, 160, 161, and 162, respectively, or SEQ ID NOs: 163, 164, 165, 166, 167, and 168, respectively; or
(13) SEQ ID NOs: 171, 172, 173, 174, 175, and 176, respectively, or SEQ ID NOs: 177, 178, 179, 180, 181, and 182, respectively;
   wherein the antibody or antigen-binding fragment thereof specifically binds IL13Ra2, preferably specifically binds human IL13Ra2.

Embodiment 2 is the isolated monoclonal antibody or antigen-binding fragment of embodiment 1, comprising a heavy chain variable region having a polypeptide sequence at least 95% identical to SEQ ID NO: 1, 15, 29, 43, 57, 71, 85, 99, 113, 127, 141, 155, or 169, or a light chain variable region having a polypeptide sequence at least 95% identical to SEQ ID NO: 2, 16, 30, 44, 58, 72, 86, 100, 114, 128, 142, 156, or 170.

Embodiment 3 is the isolated monoclonal antibody or antigen-binding fragment of embodiment 1 or 2, comprising
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:15, and a light chain variable region having the polypeptide sequence of SEQ ID NO:16;
(3) a heavy chain variable region having the polypeptide sequence of SEQ ID NO:29, and a light chain variable region having the polypeptide sequence of SEQ ID NO:30;
(4) a heavy chain variable region having the polypeptide sequence of SEQ ID NO:43, and a light chain variable region having the polypeptide sequence of SEQ ID NO:44;
(5) a heavy chain variable region having the polypeptide sequence of SEQ ID NO:57, and a light chain variable region having the polypeptide sequence of SEQ ID NO:58;
(6) a heavy chain variable region having the polypeptide sequence of SEQ ID NO:71, and a light chain variable region having the polypeptide sequence of SEQ ID NO:72;
(7) a heavy chain variable region having the polypeptide sequence of SEQ ID NO:85, and a light chain variable region having the polypeptide sequence of SEQ ID NO:86;
(8) a heavy chain variable region having the polypeptide sequence of SEQ ID NO:99, and a light chain variable region having the polypeptide sequence of SEQ ID NO:100;
(9) a heavy chain variable region having the polypeptide sequence of SEQ ID NO:113, and a light chain variable region having the polypeptide sequence of SEQ ID NO:114;
(10) a heavy chain variable region having the polypeptide sequence of SEQ ID NO:127, and a light chain variable region having the polypeptide sequence of SEQ ID NO:128;
(11) a heavy chain variable region having the polypeptide sequence of SEQ ID NO:141, and a light chain variable region having the polypeptide sequence of SEQ ID NO:142;
(12) a heavy chain variable region having the polypeptide sequence of SEQ ID NO:155, and a light chain variable region having the polypeptide sequence of SEQ ID NO:156; or
(13) a heavy chain variable region having the polypeptide sequence of SEQ ID NO:169, and a light chain variable region having the polypeptide sequence of SEQ ID NO:170.

Embodiment 4 is the isolated monoclonal antibody or antigen-binding fragment of any one of embodiments 1-3, wherein the antibody or antigen-binding fragment thereof is chimeric or human or humanized.

Embodiment 5 is the isolated monoclonal antibody or antigen-binding fragment of embodiment 4, wherein the humanized monoclonal antibody or antigen-binding fragment thereof comprises a heavy chain variable region having a polypeptide sequence at least 95%, at least 96%, at least 97%, at least 98%, or at least 99% identical to any one of SEQ ID NOs:183, 184, 185, 186, 191, 192, 193, 197, 198, 199, 203, 204, 207, 208, 211, or 212, or a light chain variable region having a polypeptide sequence at least 95%, at least 96%, at least 97%, at least 98%, or at least 99% identical to any one of SEQ ID NOs: 187, 188, 189, 190, 194, 195, 196, 200, 201, 202, 205, 206, 209, 210, or 213.

Embodiment 6 is the isolated monoclonal antibody or antigen-binding fragment thereof of embodiment 5, wherein the humanized monoclonal antibody or antigen-binding fragment thereof comprises:
(1) a heavy chain variable region having the polypeptide sequence of SEQ ID NO:183, and a light chain variable region having the polypeptide sequence of SEQ ID NO:187;
(2) a heavy chain variable region having the polypeptide sequence of SEQ ID NO:183, and a light chain variable region having the polypeptide sequence of SEQ ID NO:188;
(3) a heavy chain variable region having the polypeptide sequence of SEQ ID NO:183, and a light chain variable region having the polypeptide sequence of SEQ ID NO:189;
(4) a heavy chain variable region having the polypeptide sequence of SEQ ID NO:183, and a light chain variable region having the polypeptide sequence of SEQ ID NO:190;
(5) a heavy chain variable region having the polypeptide sequence of SEQ ID NO:184, and a light chain variable region having the polypeptide sequence of SEQ ID NO:187;
(6) a heavy chain variable region having the polypeptide sequence of SEQ ID NO:184, and a light chain variable region having the polypeptide sequence of SEQ ID NO:188;
(7) a heavy chain variable region having the polypeptide sequence of SEQ ID NO:184, and a light chain variable region having the polypeptide sequence of SEQ ID NO:189;
(8) a heavy chain variable region having the polypeptide sequence of SEQ ID NO:184, and a light chain variable region having the polypeptide sequence of SEQ ID NO:190;
(9) a heavy chain variable region having the polypeptide sequence of SEQ ID NO:185, and a light chain variable region having the polypeptide sequence of SEQ ID NO:187;
(10) a heavy chain variable region having the polypeptide sequence of SEQ ID NO:185, and a light chain variable region having the polypeptide sequence of SEQ ID NO:188;
(11) a heavy chain variable region having the polypeptide sequence of SEQ ID NO:185, and a light chain variable region having the polypeptide sequence of SEQ ID NO:189;
(12) a heavy chain variable region having the polypeptide sequence of SEQ ID NO:185, and a light chain variable region having the polypeptide sequence of SEQ ID NO:190;
(13) a heavy chain variable region having the polypeptide sequence of SEQ ID NO:186, and a light chain variable region having the polypeptide sequence of SEQ ID NO:187;
(14) a heavy chain variable region having the polypeptide sequence of SEQ ID NO:186, and a light chain variable region having the polypeptide sequence of SEQ ID NO:188;
(15) a heavy chain variable region having the polypeptide sequence of SEQ ID NO:186, and a light chain variable region having the polypeptide sequence of SEQ ID NO:189;
(16) a heavy chain variable region having the polypeptide sequence of SEQ ID NO:186, and a light chain variable region having the polypeptide sequence of SEQ ID NO:190;
(17) a heavy chain variable region having the polypeptide sequence of SEQ ID NO:191, and a light chain variable region having the polypeptide sequence of SEQ ID NO:194;
(18) a heavy chain variable region having the polypeptide sequence of SEQ ID NO:191, and a light chain variable region having the polypeptide sequence of SEQ ID NO:195;
(19) a heavy chain variable region having the polypeptide sequence of SEQ ID NO:191, and a light chain variable region having the polypeptide sequence of SEQ ID NO:196;
(20) a heavy chain variable region having the polypeptide sequence of SEQ ID NO:192, and a light chain variable region having the polypeptide sequence of SEQ ID NO:194;
(21) a heavy chain variable region having the polypeptide sequence of SEQ ID NO:192, and a light chain variable region having the polypeptide sequence of SEQ ID NO:195;
(22) a heavy chain variable region having the polypeptide sequence of SEQ ID NO:192, and a light chain variable region having the polypeptide sequence of SEQ ID NO:196;
(23) a heavy chain variable region having the polypeptide sequence of SEQ ID NO:193, and a light chain variable region having the polypeptide sequence of SEQ ID NO:194;
(24) a heavy chain variable region having the polypeptide sequence of SEQ ID NO:193, and a light chain variable region having the polypeptide sequence of SEQ ID NO:195;
(25) a heavy chain variable region having the polypeptide sequence of SEQ ID NO:193, and a light chain variable region having the polypeptide sequence of SEQ ID NO:196;
(26) a heavy chain variable region having the polypeptide sequence of SEQ ID NO:197, and a light chain variable region having the polypeptide sequence of SEQ ID NO:200;
(27) a heavy chain variable region having the polypeptide sequence of SEQ ID NO:197, and a light chain variable region having the polypeptide sequence of SEQ ID NO:201;
(28) a heavy chain variable region having the polypeptide sequence of SEQ ID NO:197, and a light chain variable region having the polypeptide sequence of SEQ ID NO:202;
(29) a heavy chain variable region having the polypeptide sequence of SEQ ID NO:198, and a light chain variable region having the polypeptide sequence of SEQ ID NO:200;
(30) a heavy chain variable region having the polypeptide sequence of SEQ ID NO:198, and a light chain variable region having the polypeptide sequence of SEQ ID NO:201;
(31) a heavy chain variable region having the polypeptide sequence of SEQ ID NO:198, and a light chain variable region having the polypeptide sequence of SEQ ID NO:202;
(32) a heavy chain variable region having the polypeptide sequence of SEQ ID NO:199, and a light chain variable region having the polypeptide sequence of SEQ ID NO:200;
(33) a heavy chain variable region having the polypeptide sequence of SEQ ID NO:199, and a light chain variable region having the polypeptide sequence of SEQ ID NO:201;
(34) a heavy chain variable region having the polypeptide sequence of SEQ ID NO:199, and a light chain variable region having the polypeptide sequence of SEQ ID NO:202;
(35) a heavy chain variable region having the polypeptide sequence of SEQ ID NO:203, and a light chain variable region having the polypeptide sequence of SEQ ID NO:205;
(36) a heavy chain variable region having the polypeptide sequence of SEQ ID NO:203, and a light chain variable region having the polypeptide sequence of SEQ ID NO:206;
(37) a heavy chain variable region having the polypeptide sequence of SEQ ID NO:204, and a light chain variable region having the polypeptide sequence of SEQ ID NO:205;
(38) a heavy chain variable region having the polypeptide sequence of SEQ ID NO:204, and a light chain variable region having the polypeptide sequence of SEQ ID NO:206;
(39) a heavy chain variable region having the polypeptide sequence of SEQ ID NO:207, and a light chain variable region having the polypeptide sequence of SEQ ID NO:209;
(40) a heavy chain variable region having the polypeptide sequence of SEQ ID NO:207, and a light chain variable region having the polypeptide sequence of SEQ ID NO:210;
(41) a heavy chain variable region having the polypeptide sequence of SEQ ID NO:208, and a light chain variable region having the polypeptide sequence of SEQ ID NO:209;
(42) a heavy chain variable region having the polypeptide sequence of SEQ ID NO:208, and a light chain variable region having the polypeptide sequence of SEQ ID NO:210;
(43) a heavy chain variable region having the polypeptide sequence of SEQ ID NO:211, and a light chain variable region having the polypeptide sequence of SEQ ID NO:213; or
(44) a heavy chain variable region having the polypeptide sequence of SEQ ID NO:212, and a light chain variable region having the polypeptide sequence of SEQ ID NO:213.

Embodiment 7 is the isolated monoclonal antibody or antigen-binding fragment of any one of embodiments 1-6, wherein the isolated antibody or antigen-binding fragment thereof is capable of inducing effector-mediated tumor cell lysis through antibody-dependent cellular cytotoxicity (ADCC), antibody-dependent cellular phagocytosis (ADCP), and/or complement-dependent cytotoxicity (CDC); and/or mediating the recruitment of conjugated drugs; and/or forming a bispecific antibody with another mAb or antigen-binding fragment thereof with cancer-killing effect.

Embodiment 8 is an isolated bispecific antibody or antigen-binding fragment thereof comprising the monoclonal antibody or antigen-binding fragment thereof of any one of embodiments 1-7.

Embodiment 9 is an isolated nucleic acid encoding the monoclonal antibody or antigen-binding fragment of any one of embodiments 1-7 or the bispecific antibody or antigen-binding fragment of embodiment 8.

Embodiment 10 is a vector comprising the isolated nucleic acid of embodiment 9.

Embodiment 11 is a host cell comprising the vector of embodiment 10.

Embodiment 12 is a pharmaceutical composition, comprising the isolated monoclonal antibody or antigen-binding fragment of any one of embodiments 1-7 or the bispecific antibody or antigen-binding fragment thereof of embodiment 8 and a pharmaceutically acceptable carrier.

Embodiment 13 is a method of targeting IL13Ra2 on a cancer cell surface, and/or treating a cancer, treating an inflammatory disease, and/or treating an autoimmune disease, in a subject in need thereof, comprising administering to the subject the pharmaceutical composition of embodiment 12, optionally, wherein the cancer is selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a hon-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 14 is a method of producing the monoclonal antibody or antigen-binding fragment of any one of embodiments 1-7 or the bispecific antibody or antigen-binding fragment thereof of embodiment 8, comprising culturing a cell comprising a nucleic acid encoding the monoclonal antibody or antigen-binding fragment thereof or bispecific antibody or antigen-binding fragment thereof under conditions to produce the monoclonal antibody or antigen-binding fragment thereof or bispecific antibody or antigen-binding fragment thereof and recovering the monoclonal antibody or antigen-binding fragment thereof or bispecific antibody or antigen-binding fragment thereof from the cell or culture.

Embodiment 15 is a method of producing a pharmaceutical composition comprising the monoclonal antibody or antigen-binding fragment thereof of any one of embodiments 1-7 or the bispecific antibody or antigen-binding fragment thereof of embodiment 8, comprising combining the monoclonal antibody or antigen-binding fragment thereof or bispecific antibody or antigen-binding fragment thereof with a pharmaceutically acceptable carrier to obtain the pharmaceutical composition.

Embodiment 16 is a method of determining the level of IL13Ra2 in a subject, the method comprising:
(1) obtaining a sample from the subject;
(2) contacting the sample with the isolated monoclonal antibody or antigen-binding fragment thereof of any one of embodiments 1-7; and
(3) determining the level of IL13Ra2 in the subject.

Embodiment 17 is the method of embodiment 16, wherein the sample is a tissue sample or a blood sample, optionally wherein the tissue sample is a cancer tissue sample.

Embodiment 18 is an isolated polynucleotide comprising a nucleic acid sequence encoding a chimeric antigen receptor (CAR), wherein the CAR comprises:
(a) an extracellular domain comprising at least one antigen binding domain that specifically binds IL13Ra2;
(b) a hinge region;
(c) a transmembrane region; and
(d) an intracellular signaling domain.

Embodiment 19 is the isolated polynucleotide of embodiment 18, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 3, 4, 5, 6, 7, and 8, respectively; or SEQ ID NOs: 9, 10, 11, 12, 13, and 14, respectively;
(2) SEQ ID NOs: 17, 18, 19, 20, 21, and 22, respectively, or SEQ ID NOs: 23, 24, 25, 26, 27, and 28, respectively;
(3) SEQ ID NOs: 31, 32, 33, 34, 35, and 36, respectively or SEQ ID NOs: 37, 38, 39, 40, 41, and 42, respectively;
(4) SEQ ID NOs: 45, 46, 47, 48, 49, and 50, respectively, or SEQ ID NOs: 51, 52, 53, 54, 55, and 56, respectively;
(5) SEQ ID NOs: 59, 60, 61, 62, 63, and 64, respectively, or SEQ ID NOs: 65, 66, 67, 68, 69, and 70, respectively;
(6) SEQ ID NOs: 73, 74, 75, 76, 77, and 78, respectively, or SEQ ID NOs: 79, 80, 81, 82, 83, and 84, respectively;
(7) SEQ ID NOs: 87, 88, 89, 90, 91, and 92, respectively, or SEQ ID NOs: 93, 94, 95, 96, 97, and 98, respectively;
(8) SEQ ID NOs: 101, 102, 103, 104, 105, and 106, respectively, or SEQ ID NOs: 107, 108, 109, 110, 111, and 112, respectively;
(9) SEQ ID NOs: 115, 116, 117, 118, 119, and 120, respectively, or SEQ ID NOs: 121, 122, 123, 124, 125, and 126, respectively;
(10) SEQ ID NOs: 129, 130, 131, 132, 133, and 134, respectively, or SEQ ID NOs: 135, 136, 137, 138, 139, and 140, respectively;
(11) SEQ ID NOs: 143, 144, 145, 146, 147, and 148, respectively, or SEQ ID NOs: 149, 150, 151, 152, 153, and 154, respectively;
(12) SEQ ID NOs: 157, 158, 159, 160, 161, and 162, respectively, or SEQ ID NOs: 163, 164, 165, 166, 167, and 168, respectively; or
(13) SEQ ID NOs: 171, 172, 173, 174, 175, and 176, respectively, or SEQ ID NOs: 177, 178, 179, 180, 181, and 182, respectively;
   wherein the antigen binding domain specifically binds IL13Ra2, preferably human IL13Ra2.

Embodiment 20 is the isolated polynucleotide of embodiment 18 or 19, wherein the antigen binding domain comprises a heavy chain variable region having a polypeptide sequence at least 95% identical to SEQ ID NO: 1, 15, 29, 43, 57, 71, 85, 99, 113, 127, 141, 155, 169, 183, 184, 185, 186, 191, 192, 193, 197, 198, 199, 203, 204, 207, 208, 211, or 212, or a light chain variable region having a polypeptide sequence at least 95% identical to SEQ ID NO: 2, 16, 30, 44, 58, 72, 86, 100, 114, 128, 142, 156, 170, 187, 188, 189, 190, 194, 195, 196, 200, 201, 202, 205, 206, 209, 210, or 213.

Embodiment 21 is the isolated polynucleotide of embodiment 20, wherein the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:15, and a light chain variable region having the polypeptide sequence of SEQ ID NO:16;
(3) a heavy chain variable region having the polypeptide sequence of SEQ ID NO:29, and a light chain variable region having the polypeptide sequence of SEQ ID NO:30;
(4) a heavy chain variable region having the polypeptide sequence of SEQ ID NO:43, and a light chain variable region having the polypeptide sequence of SEQ ID NO:44;
(5) a heavy chain variable region having the polypeptide sequence of SEQ ID NO:57, and a light chain variable region having the polypeptide sequence of SEQ ID NO:58;
(6) a heavy chain variable region having the polypeptide sequence of SEQ ID NO:71, and a light chain variable region having the polypeptide sequence of SEQ ID NO:72;
(7) a heavy chain variable region having the polypeptide sequence of SEQ ID NO:85, and a light chain variable region having the polypeptide sequence of SEQ ID NO:86;
(8) a heavy chain variable region having the polypeptide sequence of SEQ ID NO:99, and a light chain variable region having the polypeptide sequence of SEQ ID NO:100;
(9) a heavy chain variable region having the polypeptide sequence of SEQ ID NO:113, and a light chain variable region having the polypeptide sequence of SEQ ID NO:114;
(10) a heavy chain variable region having the polypeptide sequence of SEQ ID NO:127, and a light chain variable region having the polypeptide sequence of SEQ ID NO:128;
(11) a heavy chain variable region having the polypeptide sequence of SEQ ID NO:141, and a light chain variable region having the polypeptide sequence of SEQ ID NO:142;
(12) a heavy chain variable region having the polypeptide sequence of SEQ ID NO:155, and a light chain variable region having the polypeptide sequence of SEQ ID NO:156;
(13) a heavy chain variable region having the polypeptide sequence of SEQ ID NO:169, and a light chain variable region having the polypeptide sequence of SEQ ID NO:170;
(14) a heavy chain variable region having the polypeptide sequence of SEQ ID NO:183, and a light chain variable region having the polypeptide sequence of SEQ ID NO:187;
(15) a heavy chain variable region having the polypeptide sequence of SEQ ID NO:183, and a light chain variable region having the polypeptide sequence of SEQ ID NO:188;
(16) a heavy chain variable region having the polypeptide sequence of SEQ ID NO:183, and a light chain variable region having the polypeptide sequence of SEQ ID NO:189;
(17) a heavy chain variable region having the polypeptide sequence of SEQ ID NO:183, and a light chain variable region having the polypeptide sequence of SEQ ID NO:190;
(18) a heavy chain variable region having the polypeptide sequence of SEQ ID NO:184, and a light chain variable region having the polypeptide sequence of SEQ ID NO:187;
(19) a heavy chain variable region having the polypeptide sequence of SEQ ID NO:184, and a light chain variable region having the polypeptide sequence of SEQ ID NO:188;
(20) a heavy chain variable region having the polypeptide sequence of SEQ ID NO:184, and a light chain variable region having the polypeptide sequence of SEQ ID NO:189;
(21) a heavy chain variable region having the polypeptide sequence of SEQ ID NO:184, and a light chain variable region having the polypeptide sequence of SEQ ID NO:190;
(22) a heavy chain variable region having the polypeptide sequence of SEQ ID NO:185, and a light chain variable region having the polypeptide sequence of SEQ ID NO:187;
(23) a heavy chain variable region having the polypeptide sequence of SEQ ID NO:185, and a light chain variable region having the polypeptide sequence of SEQ ID NO:188;
(24) a heavy chain variable region having the polypeptide sequence of SEQ ID NO:185, and a light chain variable region having the polypeptide sequence of SEQ ID NO:189;
(25) a heavy chain variable region having the polypeptide sequence of SEQ ID NO:185, and a light chain variable region having the polypeptide sequence of SEQ ID NO:190;
(26) a heavy chain variable region having the polypeptide sequence of SEQ ID NO:186, and a light chain variable region having the polypeptide sequence of SEQ ID NO:187;
(27) a heavy chain variable region having the polypeptide sequence of SEQ ID NO:186, and a light chain variable region having the polypeptide sequence of SEQ ID NO:188;
(28) a heavy chain variable region having the polypeptide sequence of SEQ ID NO:186, and a light chain variable region having the polypeptide sequence of SEQ ID NO:189;
(29) a heavy chain variable region having the polypeptide sequence of SEQ ID NO:186, and a light chain variable region having the polypeptide sequence of SEQ ID NO:190;
(30) a heavy chain variable region having the polypeptide sequence of SEQ ID NO:191, and a light chain variable region having the polypeptide sequence of SEQ ID NO:194;
(31) a heavy chain variable region having the polypeptide sequence of SEQ ID NO:191, and a light chain variable region having the polypeptide sequence of SEQ ID NO:195;
(32) a heavy chain variable region having the polypeptide sequence of SEQ ID NO:191, and a light chain variable region having the polypeptide sequence of SEQ ID NO:196;
(33) a heavy chain variable region having the polypeptide sequence of SEQ ID NO:192, and a light chain variable region having the polypeptide sequence of SEQ ID NO:194;
(34) a heavy chain variable region having the polypeptide sequence of SEQ ID NO:192, and a light chain variable region having the polypeptide sequence of SEQ ID NO:195;
(35) a heavy chain variable region having the polypeptide sequence of SEQ ID NO:192, and a light chain variable region having the polypeptide sequence of SEQ ID NO:196;
(36) a heavy chain variable region having the polypeptide sequence of SEQ ID NO:193, and a light chain variable region having the polypeptide sequence of SEQ ID NO:194;
(37) a heavy chain variable region having the polypeptide sequence of SEQ ID NO:193, and a light chain variable region having the polypeptide sequence of SEQ ID NO:195;
(38) a heavy chain variable region having the polypeptide sequence of SEQ ID NO:193, and a light chain variable region having the polypeptide sequence of SEQ ID NO:196;
(39) a heavy chain variable region having the polypeptide sequence of SEQ ID NO:197, and a light chain variable region having the polypeptide sequence of SEQ ID NO:200;
(40) a heavy chain variable region having the polypeptide sequence of SEQ ID NO:197, and a light chain variable region having the polypeptide sequence of SEQ ID NO:201;
(41) a heavy chain variable region having the polypeptide sequence of SEQ ID NO:197, and a light chain variable region having the polypeptide sequence of SEQ ID NO:202;
(42) a heavy chain variable region having the polypeptide sequence of SEQ ID NO:198, and a light chain variable region having the polypeptide sequence of SEQ ID NO:200;
(43) a heavy chain variable region having the polypeptide sequence of SEQ ID NO:198, and a light chain variable region having the polypeptide sequence of SEQ ID NO:201;
(44) a heavy chain variable region having the polypeptide sequence of SEQ ID NO:198, and a light chain variable region having the polypeptide sequence of SEQ ID NO:202;
(45) a heavy chain variable region having the polypeptide sequence of SEQ ID NO:199, and a light chain variable region having the polypeptide sequence of SEQ ID NO:200;
(46) a heavy chain variable region having the polypeptide sequence of SEQ ID NO:199, and a light chain variable region having the polypeptide sequence of SEQ ID NO:201;
(47) a heavy chain variable region having the polypeptide sequence of SEQ ID NO:199, and a light chain variable region having the polypeptide sequence of SEQ ID NO:202;
(48) a heavy chain variable region having the polypeptide sequence of SEQ ID NO:203, and a light chain variable region having the polypeptide sequence of SEQ ID NO:205;
(49) a heavy chain variable region having the polypeptide sequence of SEQ ID NO:203, and a light chain variable region having the polypeptide sequence of SEQ ID NO:206;
(50) a heavy chain variable region having the polypeptide sequence of SEQ ID NO:204, and a light chain variable region having the polypeptide sequence of SEQ ID NO:205;
(51) a heavy chain variable region having the polypeptide sequence of SEQ ID NO:204, and a light chain variable region having the polypeptide sequence of SEQ ID NO:206;
(52) a heavy chain variable region having the polypeptide sequence of SEQ ID NO:207, and a light chain variable region having the polypeptide sequence of SEQ ID NO:209;
(53) a heavy chain variable region having the polypeptide sequence of SEQ ID NO:207, and a light chain variable region having the polypeptide sequence of SEQ ID NO:210;
(54) a heavy chain variable region having the polypeptide sequence of SEQ ID NO:208, and a light chain variable region having the polypeptide sequence of SEQ ID NO:209;
(55) a heavy chain variable region having the polypeptide sequence of SEQ ID NO:208, and a light chain variable region having the polypeptide sequence of SEQ ID NO:210;
(56) a heavy chain variable region having the polypeptide sequence of SEQ ID NO:211, and a light chain variable region having the polypeptide sequence of SEQ ID NO:213; or
(57) a heavy chain variable region having the polypeptide sequence of SEQ ID NO:212, and a light chain variable region having the polypeptide sequence of SEQ ID NO:213.

Embodiment 22 is the isolated polynucleotide of any one of embodiments 18-21, wherein the antigen binding domain is a single chain variable fragment (scFv).

Embodiment 23 is the isolated polynucleotide of embodiment 22, wherein the single chain variable fragment (scFv) is humanized.

Embodiment 24 is the isolated polynucleotide of any one of embodiments 18-23, wherein the chimeric antigen receptor (CAR) comprises one or more antigen binding domains, and/or wherein the intracellular signaling domain comprises one or more costimulatory domains and one or more activating domains.

Embodiment 25 is a chimeric antigen receptor (CAR) encoded by the isolated polynucleotide of any one of embodiments 18-24.

Embodiment 26 is a vector comprising the isolated polynucleotide of any one of embodiments 18-24.

Embodiment 27 is a host cell comprising the vector of embodiment 26, optionally, wherein the cell is a T cell or a NK cell, preferably a human T cell or a human NK cell.

Embodiment 28 is a method of making a host cell expressing a chimeric antigen receptor (CAR), the method comprising transducing a T cell or a NK cell with the vector of embodiment 26.

Embodiment 29 is a method of producing a chimeric antigen receptor (CAR)-T cell or a chimeric antigen receptor (CAR)-NK cell, the method comprising culturing T cells or NK cells comprising the isolated polynucleotide comprising a nucleic acid encoding a chimeric antigen receptor (CAR) of any one of embodiments 18-24 under conditions to produce the CAR-T cell or CAR-NK cell and recovering the CAR-T cell or CAR-NK cell.

Embodiment 30 is a method of generating a cell comprising a chimeric antigen receptor (CAR), the method comprising contacting a cell with the isolated polynucleotide comprising a nucleic acid encoding a chimeric antigen receptor (CAR) of any one of embodiments 18-24, wherein the isolated polynucleotide is an in vitro transcribed RNA or synthetic RNA.

Embodiment 31 is a method of treating cancer in a subject in need thereof, comprising administering to the subject in need thereof the host cell of embodiment 27, optionally wherein the cancer is selected from a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 32 is the method of embodiment 31, further comprising administering to the subject in need thereof an agent that increases the efficacy of a cell expressing a CAR, or an agent that ameliorates one or more side effects associated with administration of a cell expressing a CAR, or an agent that treats the disease associated with IL13Ra2.

### EXAMPLES

### Example 1: Identification of Anti-IL13Ra2 Monoclonal Antibodies

Mice were immunized with recombinant human IL13Ra2 (ACROBiosystems; Cat #: IL2-H52H5, Lot #: C333P1-97HF1-Q5) and hybridomas were produced and screened by ELISA and/or FACS. Positive clones were isolated and sequenced.

Sequences of heavy and light chain variable regions (VH and VL regions, respectively) for the anti-IL13Ra2 monoclonal antibodies are provided in Tables 1 and 2, and the CDR regions for the anti-IL13Ra2 monoclonal antibodies are provided in Tables 3-6.

TABLE 1
Sequences of heavy chain variable region for mAbs

| | | SEQ |
|---|---|---|
| mAb | | ID |
| clones | VH | NO: |
| L3A6 | QVQLQQPGAELVKPGAPVKLSCKASGYTFTNY | 1 |
| | WMNWVKQRPGRGLEWIGRIDPSDTETHYNQEV | |
| | KDKATLTVDKSSSTAYIQLSSLTSEDSAVYYC | |
| | ARGYGTYYGMDYWGQGTSVTVSS | |
| L1E1 | QVQLQQSGPELVKPGASVRLSCKASGYSFTSY | 15 |
| | YIHWMKQRPGQGLEWIGYIFPGDGSSNYNEKF | |
| | KGKATLTADTSSSTVYMQLSSLTSEDSAVYFC | |
| | ARPYYYSGRGFAYWGQGTLVTVSA | |
| L2E3 | QVQLQQSGPELVKPGASVKISCKASGYVFSRS | 29 |
| | WMNWVKQRPGKGLEWIGRIYPGDGDTNYNGKF | |
| | KGKATLTADKSSSTAYMQLSSLTSEDSAVYFC | |
| | ARGYYGNYFDYWGQGTTLTVSS | |
| L4B11 | QAQLQQSGAELVKPGASVKISCKASGYAFSDS | 43 |
| | WMSWVKQRPGKGLEWIGQIYPGDGDTNFNGNF | |
| | KGKATLTADKSSSTAYMHLSSLTSEDSAVYFC | |
| | ARRYYPVWFAYWGQGTLVTVSA | |
| L1A11 | QVTLKESGPGILQSSQTLSLTCSFSGFSLSTS | 57 |
| | GMGVSWIRQPSGMGLEWLAHIYWDDDKRYNPS | |
| | LKSRLTISKDTSRDQIFLKIASVDTSDTATYY | |
| | CARRAYDNYPYYYFDVWGAGTTVTVSS | |
| L3E3 | EVQLQQSGPALVKPGASVKISCKASGFTFTDY | 71 |
| | YMDWVKQSHGKSLEWIGYIYPNNGGTNYNRKF | |
| | KDKASLTVDKSSSTAYMELRSLTSEDSAVYYC | |
| | ARRYGNYIWFAYWGQGTLVTVSA | |
| L2A2 | QVQLQQSGPELVKPGASVKISCKASGYSFTSY | 85 |
| | YIHWVKQRPGQGLEWIGWIYPGSDNTKYNEKF | |
| | KDKATLTADTSSSTAYMQLSSLTSEDSAVYYC | |
| | ARDWENRPYFGYWGQGTTLTVSS | |
| L2F7 | QVQLKQSGAELVRPGASVKLSCKASGYTFTDS | 99 |
| | YINWVRQRPGQGLEWIARIYPKNNNTYYNEKF | |
| | KDKATLTAEKSSSTAYMQLSSLTSEDSAVYFC | |
| | ARTGGSSWAWFAYWGQGTLVTVSA | |
| L4D10 | EVKVLESGGGLVQPGGSLKLSCAASGFDFSKA | 113 |
| | WMSWVRQATGKGLEWIGEISPGSSTINYTPSL | |
| | KDKFIISRDNAKNTLYLQMSKVRSEDTALYYC | |
| | SSDYDPFAFWGQGILVTVSA | |
| S1B3 | EVQLQQSGAELVKPGASVKLSCTASGFNIKDY | 127 |
| | YMNWVKQRTEQGLEWIGRIDPEDGEIKYAPKF | |
| | QGKATITADTSSNTAYLQVSSLTSEDTAVYYC | |
| | ARPRDSSGYVGWFAYWGQGTLVTVSA | |
| L3F8 | QVQLQQSGAELMKPGASVKLSCKATGYTFTGY | 141 |
| | WIEWVKERPGHGLEWIGEILPGNGNTYYNEKF | |
| | KGKATFTADTSSNTAYMLLSSLTTEDSAIYYC | |
| | ASERRNRYAWFAYWGQGTLVTVSA | |
| L1D8 | EVQLQQSGAELVKPGASVKLSCTASGFNIKDY | 155 |
| | YMNWVKQRTEQGLEWIGRIDPEDGETKYAPKF | |
| | QGRATITADTSSNTAYLQLGSLTSEDTAVYYC | |
| | ARPLASSGYVGWFAYWGQGTLVTVSS | |
| L2H3 | QVQLQQSGAELMKPGASVKLSCKATGYTFTGY | 169 |
| | WIEWLKQRPGHGLEWIGEILPGNGSTYYNEKF | |
| | KGKATFTADTSSNTAYMQLSSLTTEDSAIYYC | |
| | ATEEYGNYEAWFSYWGQGTLVTVSA | |

VH: heavy chain variable region

TABLE 2
Sequences of light chain variable region for mAbs

| | | SEQ |
|---|---|---|
| mAb | | ID |
| clones | VL | NO: |
| L3A6 | DIVLTQSPASLAVSLGQRATISCRASESVDNYGIS | 2 |
| | FMNWFQQKPGQPPKLLIYAASNQGSGVPARFSGSG | |
| | SGTDFSLNIHPLEEDDTALYFCQQSKEVPWTFGGG | |
| | TKLEIK | |
| L1E1 | DIVLTQSPATLSVTPGDRVSLSCRASQSISNYLHW | 16 |
| | YQQKSHESPRLLIKSASQSISGIPSRFSGSGSGTD | |
| | FTLSINSVETEDFGMYFCQQSNSWPLTFGAGTKLE | |
| | LK | |
| L2E3 | DIVMTQAAFSNPVTLGTSASISCRSSKSLLHTDGI | 30 |
| | TYLYWYLQRPGQSPQLLIYWMSNLASGVPDRFSGS | |
| | GSGTDFTLRISRVEAEDVGVYYCAQILEFPRTFGG | |
| | GTKLEIK | |
| L4B11 | DVLMTQTPLSLPVSLGDQASISCRSSQSIVHSNGN | 44 |
| | TYLEWYLQKPGQSPKPLIYKVSNRFSGVPDRFSGS | |
| | GSGTDFTLKISRVEAEDLGVYYCFQSSHVPFTFGS | |
| | GTKLEIK | |
| L1A11 | DIQMTQTTSSLSASLGDRVTISCRASQDISNYLNW | 58 |
| | YQQKPDGTVKLLIYYTSRLHSGVPSRFSGSGSGTD | |
| | YSLTISNLEPEDIATYYCQQYGKLFTFGTGTKLEI | |
| | K | |
| L3E3 | DVVMTQTPLSLPVSLGDQASISCRSSQSLVHSNGN | 72 |
| | TYLHWYLQKPGQSPKLLIYKVSNRFSGVPDRFSGS | |
| | GSGTDFTLKISRVEAEDLGVYFCSQSTHVPTFGGG | |
| | TKLEIK | |
| L2A2 | DIVLIQSPATLSVTPGDRVSLSCRASQSISSYLHW | 86 |
| | YQQKSHESPRLLIKYASQSISGIPSRFSGSGSGTD | |
| | FTLSINSVETEDFGMYFCQQSNSWPLTFGAGTKLV | |
| | LK | |
| L2F7 | DIQMTQSPASLAASVGETITITCQASENIYFSLAW | 100 |
| | YQQKQGKSPQLLIYNANSLEDGVPSRFSGSGSGTQ | |
| | FSMKINSMQPEDTATYFCKQAYDFPYTFGGGTKLE | |
| | IK | |
| L4D10 | DVLMTQTPLSLPVSLGDHASISCRSSQTIVHSNGN | 114 |
| | TYLEWYLQKPGQSPKLLIYKVSNRFSGVPDRFSGS | |
| | GSGTDFTLKISRVEAEDLGVYYCFQGSHVPFTFGS | |
| | GTKLEIK | |
| S1B3 | QIVLTQSPAIMSASPGEKVTMTCSASSTVSSSYLH | 128 |
| | WYQQKSGASPKLWIYGTSNLASGVPARFSGSGSGT | |
| | SYSLTISGVEAEDAATYYCQQYHSDPPTFGGGTKL | |
| | EIK | |
| L3F8 | DVVMTQTPLSLSVTIGQPASISCKSSQSLLYSDGK | 142 |
| | TYLTWLQQRPGQSPKRLMYQVSKLDPGIPDRFSGS | |
| | GSETDFSLKISRVEAEDLGVYYCLQGTYYPRTFGG | |
| | GTKLEIK | |
| L1D8 | QIVLTQSPAIMSASPGEKVTMTCSASSSVSSRYLH | 156 |
| | WYQQKSGASPKLWIYVTSNLASGVPARFSGSGSGT | |
| | SYSLTISSVEAEDAATYYCQQYHSAPPTFGGGTKL | |
| | EIK | |
| L2H3 | DVVMTQTPLSLSVTIGQPASISCKSSQSLLYSDGK | 170 |
| | TYLNWLQQRPGQSPKRLMYQVSKLDPGIPDRFSGS | |
| | GSETDFTLKISRVEAEDLGVYYCLQGTYYPQTFGS | |
| | GTKLEIK | |

VL: light chain variable region

TABLE 3
CDR regions 1-3 of heavy chain for mAbs

| mAb | | | | | | |
|---|---|---|---|---|---|---|
| clones | HC CDR1 | NO | HC CDR2 | NO | HC CDR3 | NO |
| L3A6 | GYTFTNYW | 3 | IDPSDTET | 4 | ARGYGTYY | 5 |
| | | | | | GMDY | |
| L1E1 | GYSFTSYY | 17 | IFPGDGSS | 18 | ARPYYYSG | 19 |
| | | | | | RGFAY | |
| L2E3 | GYVFSRSW | 31 | IYPGDGDT | 32 | ARGYYGNY | 33 |
| | | | | | FDY | |
| L4B11 | GYAFSDSW | 45 | IYPGDGDT | 46 | ARRYYPVW | 47 |
| | | | | | FAY | |
| L1A11 | GFSLSTSGMG | 59 | IYWDDDK | 60 | ARRAYDNY | 61 |
| | | | | | PYYYFDV | |
| L3E3 | GFTFTDYY | 73 | IYPNNGGT | 74 | ARRYGNYI | 75 |
| | | | | | WFAY | |
| L2A2 | GYSFTSYY | 87 | IYPGSDNT | 88 | ARDWENRP | 89 |
| | | | | | YFGY | |
| L2F7 | GYTFTDSY | 101 | IYPKNNNT | 102 | ARTGGSSW | 103 |
| | | | | | AWFAY | |
| L4D10 | GFDFSKAW | 115 | ISPGSSTI | 116 | SSDYDPFA | 117 |
| | | | | | F | |
| S1B3 | GFNIKDYY | 129 | IDPEDGEI | 130 | ARPRDSSG | 131 |
| | | | | | YVGWFAY | |
| L3F8 | GYTFTGYW | 143 | ILPGNGNT | 144 | ASERRNRY | 145 |
| | | | | | AWFAY | |
| L1D8 | GFNIKDYY | 157 | IDPEDGET | 158 | ARPLASSG | 159 |
| | | | | | YVGWFAY | |
| L2H3 | GYTFTGYW | 171 | ILPGNGST | 172 | ATEEYGNY | 173 |
| | | | | | EAWFSY | |

HC: heavy chain;
CDR: complementarity determining region;
NO: SEQ ID NO
The HC CDRs for the anti-IL13Ra2 mAbs were determined utilizing the IMGT method (Lefranc, M.-P. et al., Nucleic Acids Res. 1999; 27:209-212).

TABLE 4
CDR regions 1-3 of light chain for mAbs

| mAb | | | | | | |
|---|---|---|---|---|---|---|
| clones | LC CDR1 | NO | LC CDR2 | NO | LC CDR3 | NO |
| L3A6 | ESVDNYGISF | 6 | AAS | 7 | QQSKEVPWT | 8 |
| L1E1 | QSISNY | 20 | SAS | 21 | QQSNSWPLT | 22 |
| L2E3 | KSLLHTDGITY | 34 | WMS | 35 | AQILEFPRT | 36 |
| L4B11 | QSIVHSNGNTY | 48 | KVS | 49 | FQSSHVPFT | 50 |
| L1A11 | QDISNY | 62 | YTS | 63 | QQYGKLFT | 64 |
| L3E3 | QSLVHSNGNTY | 76 | KVS | 77 | SQSTHVPT | 78 |
| L2A2 | QSISSY | 90 | YAS | 91 | QQSNSWPLT | 92 |
| L2F7 | ENIYFS | 104 | NAN | 105 | KQAYDFPYT | 106 |
| L4D10 | QTIVHSNGNTY | 118 | KVS | 119 | FQGSHVPFT | 120 |
| S1B3 | STVSSSY | 132 | GTS | 133 | QQYHSDPPT | 134 |
| L3F8 | QSLLYSDGKTY | 146 | QVS | 147 | LQGTYYPRT | 148 |
| L1D8 | SSVSSRY | 160 | VTS | 161 | QQYHSAPPT | 162 |
| L2H3 | QSLLYSDGKTY | 174 | QVS | 175 | LQGTYYPQT | 176 |

LC: light chain;
CDR: complementarity determining region;
NO: SEQ ID NO
The LC CDRs for the anti-IL13Ra2 mAbs were determined utilizing the IMGT method (Lefranc, M.-P. et al., Nucleic Acids Res. 1999; 27:209-212).

TABLE 5
CDR regions 1-3 of heavy chain for mAbs

| mAb | | | | | | |
|---|---|---|---|---|---|---|
| clones | HC CDR1 | NO | HC CDR2 | NO | HC CDR3 | NO |
| L3A6 | NYWMN | 9 | RIDPSDTET | 10 | GYGTYYGMDY | 11 |
| | | | HYNQEVKD | | | |
| L1E1 | SYYIH | 23 | YIFPGDGSS | 24 | PYYYSGRGFAY | 25 |
| | | | NYNEKFKG | | | |
| L2E3 | RSWMN | 37 | RIYPGDGDT | 38 | GYYGNYFDY | 39 |
| | | | NYNGKFKG | | | |
| L4B11 | DSWMS | 51 | QIYPGDGDT | 52 | RYYPVWFAY | 53 |
| | | | NFNGNFKG | | | |
| L1A11 | TSGMGVS | 65 | HIYWDDDKR | 66 | RAYDNYPYYYFDV | 67 |
| | | | YNPSLKS | | | |
| L3E3 | DYYMD | 79 | YIYPNNGGT | 80 | RYGNYIWFAY | 81 |
| | | | NYNRKFKD | | | |
| L2A2 | SYYIH | 93 | WIYPGSDNT | 94 | DWENRPYFGY | 95 |
| | | | KYNEKFKD | | | |
| L2F7 | DSYIN | 107 | RIYPKNNNT | 108 | TGGSSWAWFAY | 109 |
| | | | YYNEKFKD | | | |
| L4D10 | KAWMS | 121 | EISPGSSTI | 122 | DYDPFAF | 123 |
| | | | NYTPSLKD | | | |
| S1B3 | DYYMN | 135 | RIDPEDGEI | 136 | PRDSSGYVGWFAY | 137 |
| | | | KYAPKFQG | | | |
| L3F8 | GYWIE | 149 | EILPGNGNT | 150 | ERRNRYAWFAY | 151 |
| | | | YYNEKFKG | | | |
| L1D8 | DYYMN | 163 | RIDPEDGET | 164 | PLASSGYVGWFAY | 165 |
| | | | KYAPKFQG | | | |
| L2H3 | GYWIE | 177 | EILPGNGST | 178 | EEYGNYEAWFSY | 179 |
| | | | YYNEKFKG | | | |

HC: heavy chain;
CDR: complementarity determining region;
NO: SEQ ID NO
The HC CDRs for the anti-IL13Ra2 mAbs were determined utilizing the Kabat method (Elvin A. Kabat et al, Sequences of Proteins of Immunological Interest 5th ed. 1991).

TABLE 6
CDR regions 1-3 of light chain for mAbs

| mAb | | | | | | |
|---|---|---|---|---|---|---|
| clones | LC CDR1 | NC | LC CDR2 | NO | LC CDR3 | NO |
| L3A6 | RASESVDN | 12 | AASNQGS | 13 | QQSKEVPWT | 14 |
| | YGISFMN | | | | | |
| L1E1 | RASQSISN | 26 | SASQSIS | 27 | QQSNSWPLT | 28 |
| | YLH | | | | | |
| L2E3 | RSSKSLLH | 40 | WMSNLAS | 41 | AQILEFPRT | 42 |
| | TDGITYLY | | | | | |
| L4B11 | RSSQSIVH | 54 | KVSNRFS | 55 | FQSSHVPFT | 56 |
| | SNGNTYLE | | | | | |
| L1A11 | RASQDISN | 68 | YTSRLHS | 69 | QQYGKLFT | 70 |
| | YLN | | | | | |
| L3E3 | RSSQSLVH | 82 | KVSNRFS | 83 | SQSTHVPT | 84 |
| | SNGNTYLH | | | | | |
| L2A2 | RASQSISS | 96 | YASQSIS | 97 | QQSNSWPLT | 98 |
| | YLH | | | | | |
| L2F7 | QASENIYF | 110 | NANSLED | 111 | KQAYDFPYT | 112 |
| | SLA | | | | | |
| L4D10 | RSSQTIVH | 124 | KVSNRFS | 125 | FQGSHVPFT | 126 |
| | SNGNTYLE | | | | | |
| S1B3 | SASSTVSS | 138 | GTSNLAS | 139 | QQYHSDPPT | 140 |
| | SYLH | | | | | |
| L3F8 | KSSQSLLY | 152 | QVSKLDP | 153 | LQGTYYPRT | 154 |
| | SDGKTYLT | | | | | |
| L1D8 | SASSSVSS | 166 | VTSNLAS | 167 | QQYHSAPPT | 168 |
| | RYLH | | | | | |
| L2H3 | KSSQSLLY | 180 | QVSKLDP | 181 | LQGTYYPQT | 182 |
| | SDGKTYLN | | | | | |

LC: light chain;
CDR: complementarity determining region;
NO: SEQ ID NO
The LC CDRs for the anti-IL13Ra2 mAbs were determined utilizing the Kabat method (Elvin A. Kabat et al, Sequences of Proteins of Immunological Interest 5th ed. 1991).

### Example 2: Production and Purification of mAbs from Culture Media of Transfected Cells

To obtain recombinant anti-IL13Ra2 chimeric mAbs, the expression vectors containing the mouse variable regions (VH and VL) fused to the constant regions of human IgG1 heavy chain and kappa light chain, respectively, were transiently transfected into ExpiCHO-S cells. The recombinant antibodies produced in the suspension of the ExpiCHO-S cells were purified using Protein A affinity chromatography.

### Example 3: ELISA Binding Analysis of Purified Chimeric mAbs

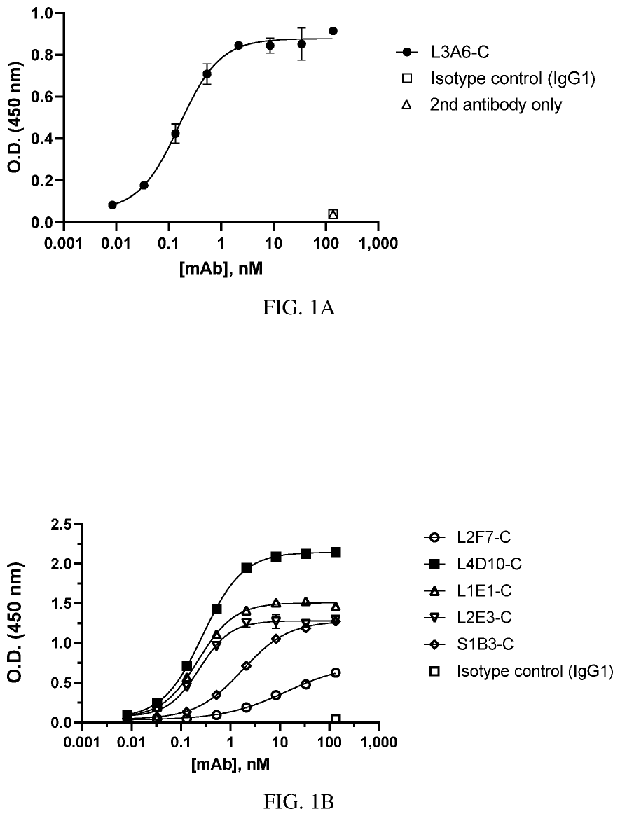

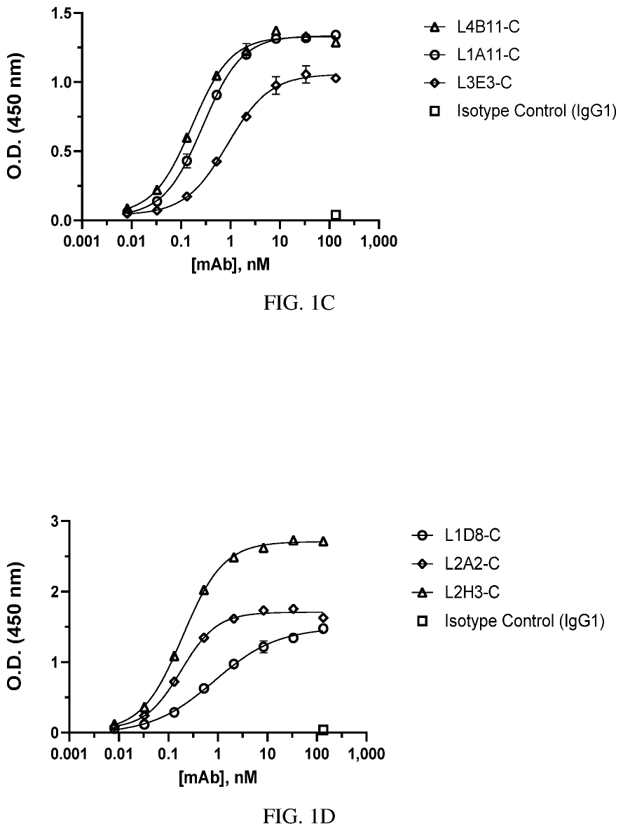

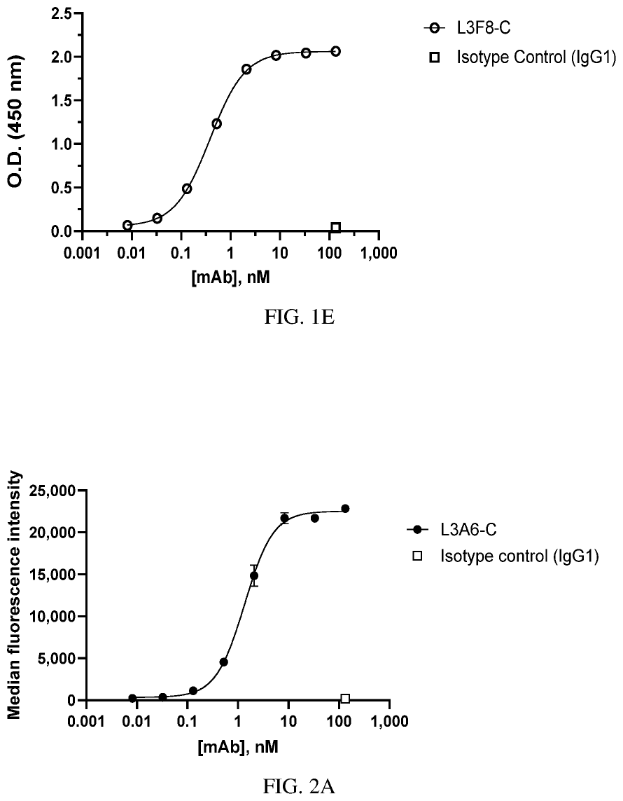

Purified chimeric mAbs were tested in an ELISA assay for their ability to bind to immobilized IL13Ra2. Recombinant human IL13Ra2 in Carbonate-Bicarbonate Buffer (Sigma; St. Louis, MO; Cat #: C3041-100CAP) was coated on a 96-well plate at room temperature for 1 hour. The plate is blocked by 5% BSA in TBST for 1 hour at room temperature. In each well of an individual plate, mAbs at various concentrations were incubated for 1 hour at room temperature. The plate was washed and the binding to IL13Ra2 was detected by a mouse anti-human IgG Fc antibody conjugated to horseradish peroxidase (Genescript; Piscataway, NJ; Cat #: A01854) with incubation for 1 hour at room temperature. Then after washing, the ELISA was developed using One-step Detection Solution (ThermoFisher Scientific; Waltham, MA; Cat #: 34028) and measured as the absorbance at 450 nm. The result is shown in FIGS. 1A-1E. L3A6-C is the chimeric version of L3A6, with other antibodies following the same naming rule.

### Example 4: FACS Binding Analysis of Purified Chimeric mAbs

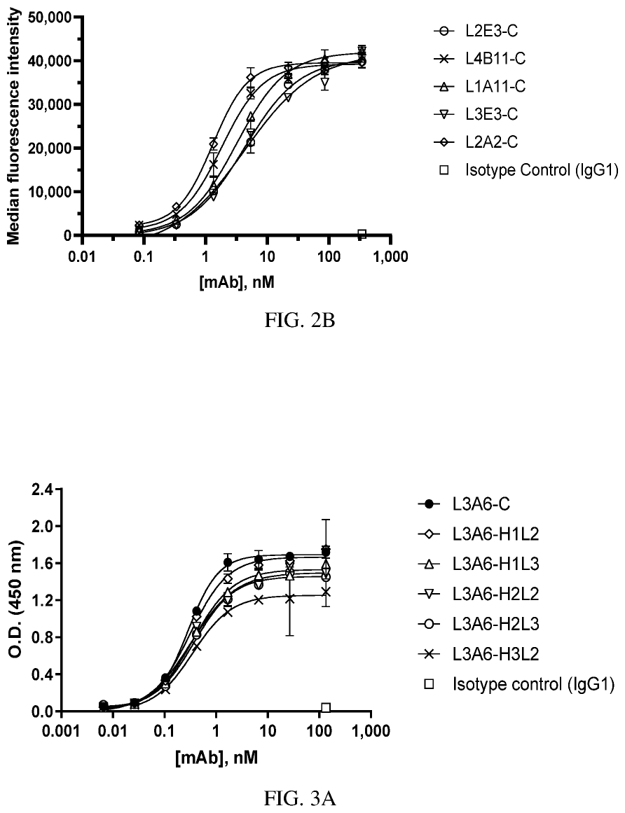

A375 cells were transferred to a 96-well plate. Around 50,000 cells were incubated with purified chimeric anti-IL13Ra2 mAbs (variable regions of mouse mAbs fused to the constant regions of human IgG1 heavy chain and kappa light chain, respectively) at various concentrations for 15 minutes at 4° C. Cells were then centrifuged for 5 minutes and washed once with FACS buffer (HBSS supplemented with 5% BSA and 0.05% sodium azide). The cells were then incubated with PE/Cyanine7 labeled anti-human IgG Fc recombinant antibody (Biolegend; San Diego, CA; Cat #366907) or F(ab′)2-Goat anti-Human IgG Fc Secondary Antibody, Alexa Fluor™ 488 (ThermoFisher; Cat #H10120) and incubated on ice for another 15 minutes. Cells were then washed with FACS buffer once and resuspended in FACS buffer. Cells were then run through the Attune N×T and the data were analyzed by the Attune N×T software. The result is shown in FIGS. 2A-2B. L3A6-C is the chimeric version of L3A6, with other antibodies following the same naming rule.

### Example 5: Humanization of Anti-IL13Ra2 mAbs

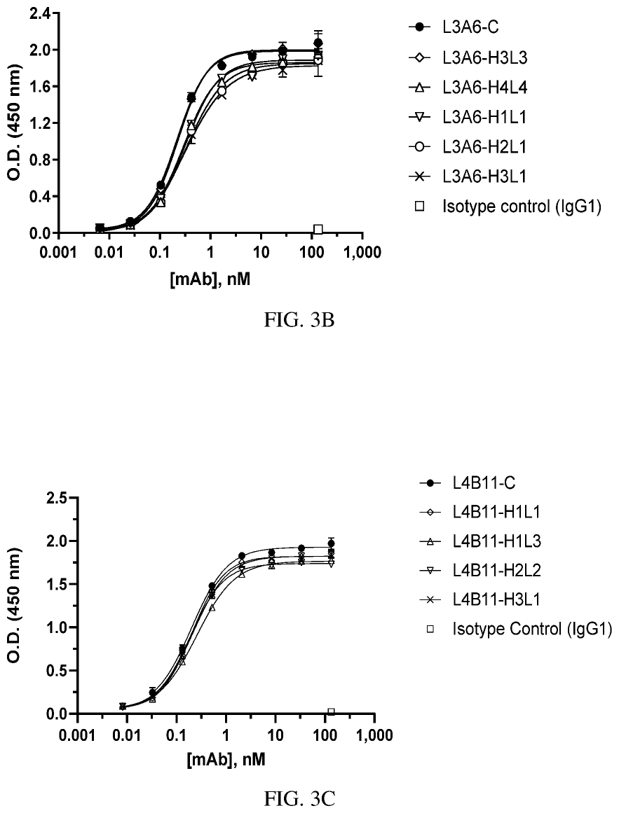

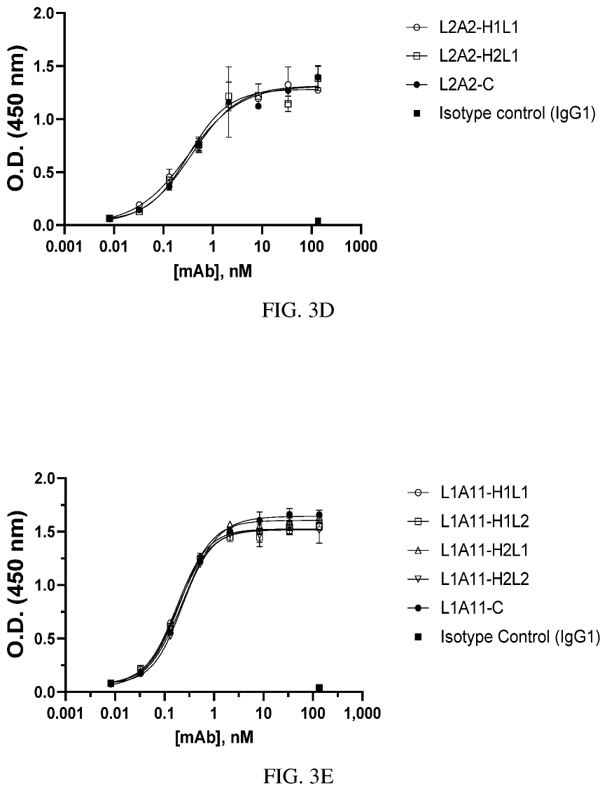

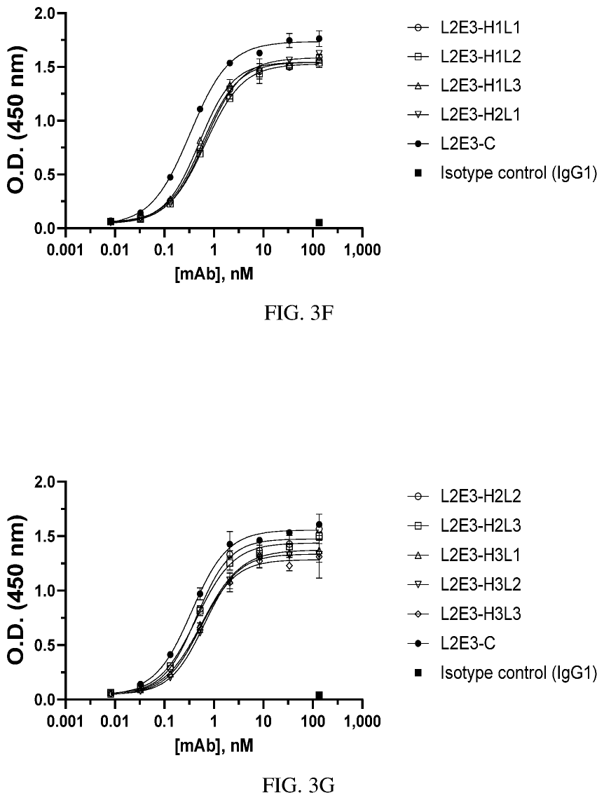

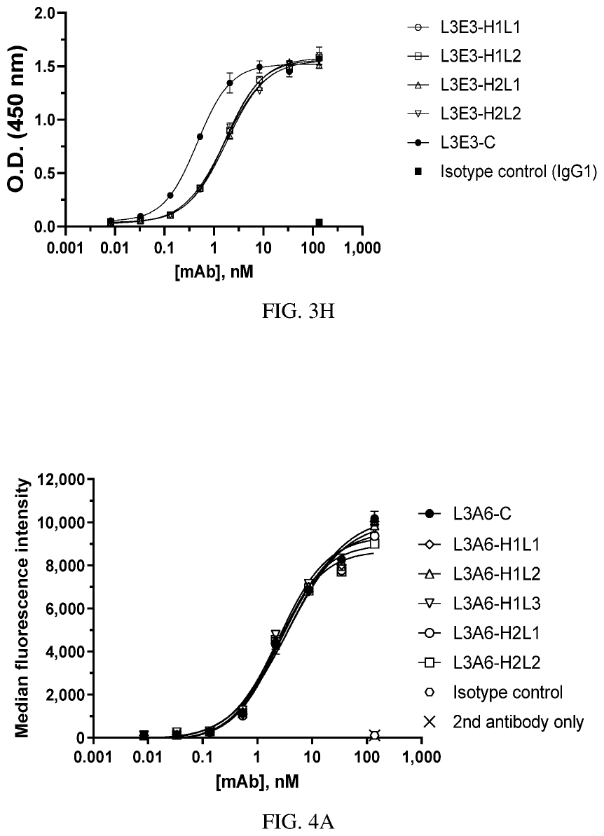

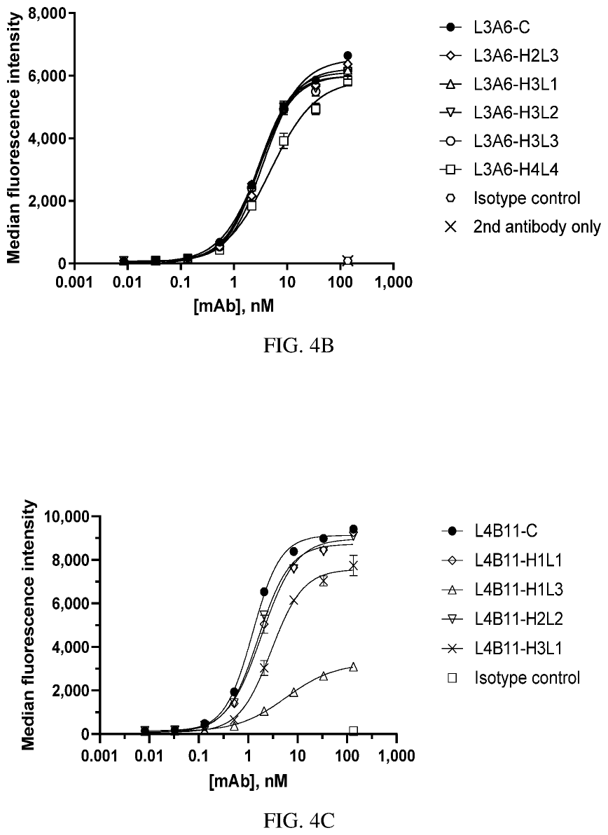

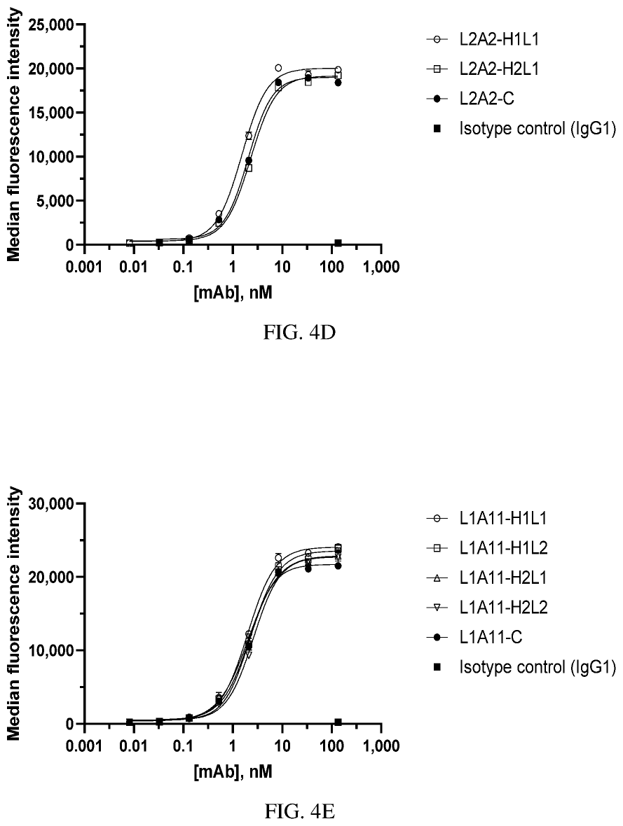

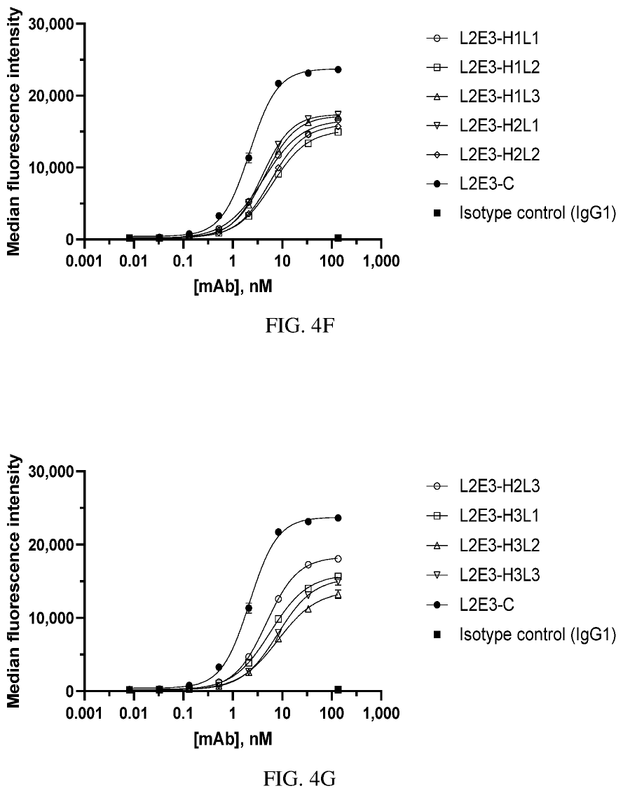

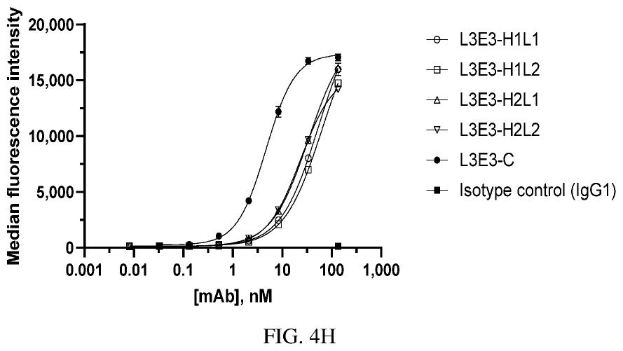

The mouse anti-IL13Ra2 mAbs were humanized to reduce the potential of immunogenicity when used in human patients. The sequences of the variable regions of the heavy and light chains (VH and VL) were compared with the human antibody sequences in the Protein Data Bank (PDB) database and homology models were built. The CDRs in both the heavy and light chains of the mouse mAbs were grafted into human frameworks that have the highest possibility of maintaining the proper structure likely required for antigen binding. Backmutations from human residues to mouse residue or other mutations were designed when necessary. The sequences of the humanized VH and VL regions are shown in Table 7. The humanized VH and VL regions were fused to the constant regions of human IgG1 heavy chain and kappa light chain, respectively. The binding of the humanized mAbs to IL13Ra2 were assessed using ELISA assay (FIGS. 3A-3H) and FACS assay (FIGS. 4A-4H). L3A6-H1L1 refers to the humanized mAb constructed with L3A6-H1 heavy chain and L3A6-L1 light chain shown in Table 7; other humanized clones follow the same naming rule.

TABLE 7
Sequences of heavy chain and light chain variable
regions of humanized anti-IL13Ra2 mAbs

| VH/VL | SEQUENCE | NO |
|---|---|---|
| L3A6-H1 | QVTLRESGPALVKPTQTLTLTCTASGYTFTNYWMNWVRQPPGKALEWIGRIDP | 183 |
| | SDTETHYNQEVKDRATLSVDKSKNQAVLTMTNMDPVDTATYYCARGYGTYY | |
| | GMDYWGQGSLVTVSS | |
| L3A6-H2 | EVTLKESGPTLVKPTQTLTLTCTASGYTFTNYWMNWVRQPPGKALEWIGRIDP | 184 |
| | SDTETHYNQEVKDRATLTVDKSKNQAVLTMTNMDPVDTATYYCARGYGTYY | |
| | GMDYWGQGTLVTVSS | |
| L3A6-H3 | EVTLKESGPTLVKPTQTLTLTCKASGYTFTNYWMNWVRQPPGKALEWIGRIDP | 185 |
| | SDTETHYNQEVKDRATLTVDTSKNQAVLTMTNMDPVDTATYYCARGYGTYY | |
| | GMDYWGQGTLVTVSS | |
| L3A6-H4 | EVQLVQSGAELKKPGSSVKVSCKASGYTFTNYWMNWVKQAPGQGLEWIGRID | 186 |
| | PSDTETHYNQEVKDKATLTVDKSTNTAYMELSSLRSEDTAVYYCARGYGTYY | |
| | GMDYWGQGTLVTVSS | |
| L3A6-L1 | DIVLTQSPDSLSVSLGERATINCRASESVDNYGISFMNWFQQKPGQPPKLLIYAA | 187 |
| | SNQGSGVPDRFSGSGSGTDFTLTISSLQAEDVAVYFCQQSKEVPWTFGGGTKVE | |
| | IK | |
| L3A6-L2 | DIVLTQSPDSLAVSLGERATINCRASESVDNYGISFMNWFQQKPGQPPKLLIYA | 188 |
| | ASNQGSGVPDRFSGSGSGTDFTLTISSLQAEDVAVYFCQQSKEVPWTFGQGTK | |
| | VEIK | |
| L3A6-L3 | DIVLTQSPASLAVSLGERATINCRASESVDNYGISFMNWFQQKPGQPPKLLIYA | 189 |
| | ASNQGSGVPDRFSGSGSGTDFTLTISSLQAEDVAVYFCQQSKEVPWTFGQGTK | |
| | VEIK | |
| L3A6-L4 | DIQLTQSPSTLSASVGDRVTITCRASESVDNYGISFMNWFQQKPGKAPKLLIYA | 190 |
| | ASNQGSGVPSRFSGSGSGTDFTLTISSLQPDDFATYFCQQSKEVPWTFGQGTKV | |
| | EVK | |
| L4B11-H1 | QAQLVQSGPEVKKPGTSVRVSCKASGYAFSDSWMSWVRQARGQRLEWIGQIY | 191 |
| | PGDGDTNFNGNFKGRATLTADKSTSTAYMELSSLRSEDTAVYFCARRYYPVWF | |
| | AYWGQGTMVTVSS | |
| L4B11-H2 | EVQLLESGGGLVQPGGSLRLSCAASGYAFSDSWMSWVRQAPGKGLEWIGQIYP | 192 |
| | GDGDTNFNGNFKGRATLSADKSKNTAYLQMNSLRAEDTAVYFCARRYYPVW | |
| | FAYWGQGTMVTVSS | |
| L4B11-H3 | QVQLEQSGAEVKKPGSSVKVSCKASGYAFSDSWMSWVRQAPGQGLEWIGQIY | 193 |
| | PGDGDTNFNQRFQGRATLTADKSMTTAYMELSSLRSEDTAMYFCARRYYPVW | |
| | FAYWGQGTMVTVSS | |
| L4B11-L1 | DVVMTQTPGTLSLSPGERATLSCRSSQSIVHSNGNTYLEWYQQKPGQAPRPLIY | 194 |
| | KVSNRFSGIPDRFSGSGSGTDFTLTISRLEPEDFAVYYCFQSSHVPFTFGQGTKVE | |
| | IK | |
| L4B11-L2 | DVQMTQSPSTLSASVGDRVTITCRSSQSIVHSNGNTYLEWYQQKPGKAPKPLIY | 195 |
| | KVSNRFSGVPSRFSGSGSGTEFTLTISSLQPDDFATYYCFQSSHVPFTFGQGTKV | |
| | EIK | |
| L4B11-L3 | EVEMTQSPATLSVSPGESATLSCRSSQSIVHSNGNTYLEWYQQKPGQAPRPLIY | 196 |
| | KVSNRAPAIPGRFSGSGSGTDFTLTISRLEPEDLAVYYCFQSSHVPFTFGQGTKV | |
| | EIK | |
| L2E3-H1 | QVQLQESGPGLVKPSETLSLTCTASGYVFSRSWMNWVRQSPGKGLEWIGRIYP | 197 |
| | GDGDTNYNGKFKGRATLSADKSKTQASLKLSSVTAADTAIYFCARGYYGNYF | |
| | DYWGQGTMVTVSS | |
| L2E3-H2 | QVQLVESGPGLVKPSQTLSLTCTASGYVFSRSWMNWVRQPAGKGLEWIGRIYP | 198 |
| | GDGDTNYNGKFKGRATLSADKSKNQASLKLSSVTAADTAVYFCARGYYGNYF | |
| | DYWGKGTLVTVSS | |
| L2E3-H3 | EVTLKESGPVLVKPTETLTLTCTASGYVFSRSWMNWVRQPPGKALEWIGRIYP | 199 |
| | GDGDTNYNGKFKGRATLSADKSKSQAVLTMTNMDPVDTATYFCARGYYGNY | |
| | FDYWGQGTLVTVSS | |
| L2E3-L1 | DIQMTQSPSSLSASVGDRVTITCRSSKSLLHTDGITYLYWYQQKPGKAPKLLIY | 200 |
| | WMSNLASGVPSRFSGSGSGTDFTFTISSLQPEDIATYYCAQILEFPRTFGGGTKV | |
| | EIK | |
| L2E3-L2 | DIVMTQSPSSLSASVGDRVTITCRSSKSLLHTDGITYLYWYQQKPGRAPDLLIY | 201 |
| | WMSNLASGVPSRFSGSGSGTDFTLTISSLQPEDFAVYYCAQILEFPRTFGQGTKL | |
| | QIK | |
| L2E3-L3 | EIVMTQSPATLSLSPGERATLSCRSSKSLLHTDGITYLYWYQQKPGQAPRLLIY | 202 |
| | WMSNLASGIPARFSGSGSGTDFTLTISSLEPEDFAVYYCAQILEFPRTFGQGTKL | |
| | EIK | |
| L1A11-H1 | QVQLQESGPGLVKPSETLSLTCTFSGFSLSTSGMGVSWIRQSPGKGLEWLAHIY | 203 |
| | WDDDKRYNPSLKSRLTISKDTSKTQISLKLSSVTAADTAIYYCARRAYDNYPYY | |
| | YFDVWGQGTMVTVSS | |
| L1A11-H2 | QVTLRESGPALVKPTQTLTLTCTFSGFSLSTSGMGVSWIRQPPGKALEWLAHIY | 204 |
| | WDDDKRYNPSLKSRLTISKDTSKNQIVLTMTNMDPVDTATYYCARRAYDNYP | |
| | YYYFDVWGQGSLVTVSS | |
| L1A11-L1 | DIQMTQSPSSLSASVGDRVTITCRASQDISNYLNWYQQKPGKAVKLLIYYTSRL | 205 |
| | HSGVPSRFSGSGSGTDYTFTISSLQPEDIATYYCQQYGKLFTFGGGTKVEIK | |
| L1A11-L2 | DIVMTQSPDSLSVSLGERATINCRASQDISNYLNWYQQKPGQPVKLLIYYTSRL | 206 |
| | HSGVPDRFSGSGSGTDYTLTISSLQAEDVAVYYCQQYGKLFTFGGGTKVEIK | |
| L3E3-H1 | EVTLKESGPTLVKPTQTLTLTCTASGFTFTDYYMDWVRQPPGKALEWIGYIYPN | 207 |
| | NGGTNYNRKFKDRATLTVDKSKNQAVLTMTNMDPVDTATYYCARRYGNYIW | |
| | FAYWGQGTLVTVSS | |
| L3E3-H2 | QVQLQESGPGLVKPSQTLSLTCTASGFTFTDYYMDWVRQLPGKGLEWIGYIYP | 208 |
| | NNGGTNYNRKFKDRATLSVDKSKKQASLRLSSVTAADTAVYYCARRYGNYIW | |
| | FAYWGQGTTVTVSS | |
| L3E3-L1 | DVVMTQSPDSLAVSLGERATINCRSSQSLVHSNGNTYLHWYQQKPGQPPKLLI | 209 |
| | YKVSNRFSGVPDRFSGSGSGTDFTLTISSLQAEDVAVYFCSQSTHVPTFGQGTK | |
| | VEIK | |
| L3E3-L2 | EVVMTQSPGTLSLSPGERATLSCRSSQSLVHSNGNTYLHWYQQKPGQAPSLLIY | 210 |
| | KVSNRFSGIPDRFSGSGSGTDFTLTISRLEPEDFAVYFCSQSTHVPTFGQGTKVEI | |
| | K | |
| L2A2-H1 | QVQLVQSGAEVKKPGSSVKVSCKASGYSFTSYYIHWVRQAPGQGLEWIGWIYP | 211 |
| | GSDNTKYNEKFKDRATLTADTSTSTAYMELSSLRSEDTAVYYCARDWENRPYF | |
| | GYWGQGTLVTVSS | |
| L2A2-H2 | QVQLEESGPGLVKPSETLSLTCSASGYSFTSYYIHWVRQPPGKGLEWIGWIYPG | 212 |
| | SDNTKYNEKFKDRATLSADTSKNEASLRLTSVTAADTAVYYCARDWENRPYF | |
| | GYWGQGTLVTVSS | |
| L2A2-L1 | DIQLTQSPSSVSASVGDRVTITCRASQSISSYLHWYQQKPGKAPKLLIKYASQSIS | 213 |
| | GVPSRFSGSGSGTDFTLTISSLQPEDFATYFCQQSNSWPLTFGQGTKVEIK | |

NO: SEQ ID NO.

### Example 6: Construction of Chimeric Antigen Receptor Constructs Comprising Anti-IL13Ra2 Antigen Binding Domains

To construct CAR constructs, the mAbs are converted into scFvs using the VH, VL, and a (G4S)n linker, and the scFv is fused to the N-terminus of the hinge and transmembrane domains derived from human CD8a (aa 114-118, Boursier J P et al., J. Biol. Chem. 268(3):2013-20 (1993)). The C-terminal intracellular signaling domain of the CAR is constructed by fusing the intracellular costimulatory domain of CD28 (aa 162-202, Aruffo A and Seed B, Proc. Natl. Acad. Sci. USA 84(23):8573-7 (1987)) followed by the activation domain from CD3 zeta chain (aa 52-162, Letourneur F and Klausner R D, Proc. Natl. Acad. Sci. USA 88(20):8905-9 (1991)). The DNA sequence encoding the CAR is assembled and cloned into an expression vector (either retroviral, lentiviral, extrachromosomal or integrated) to generate the CAR construct using standard molecule biology cloning techniques.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the scope of the present invention as defined by the present description.

1. An isolated monoclonal antibody or antigen-binding fragment or an antigen-binding domain thereof comprising a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 3, 4, 5, 6, 7 and 8, respectively; or SEQ ID NOs: 9, 10, 11, 12, 13 and 14, respectively;
(2) SEQ ID NOs: 17, 18, 19, 20, 21, and 22, respectively, or SEQ ID NOs: 23, 24, 25, 26, 27, and 28, respectively;
(3) SEQ ID NOs: 31, 32, 33, 34, 35, and 36, respectively or SEQ ID NOs: 37, 38, 39, 40, 41, and 42, respectively;
(4) SEQ ID NOs: 45, 46, 47, 48, 49, and 50, respectively, or SEQ ID NOs: 51, 52, 53, 54, 55, and 56, respectively;
(5) SEQ ID NOs: 59, 60, 61, 62, 63, and 64, respectively, or SEQ ID NOs: 65, 66, 67, 68, 69, and 70, respectively;
(6) SEQ ID NOs: 73, 74, 75, 76, 77, and 78, respectively, or SEQ ID NOs: 79, 80, 81, 82, 83, and 84, respectively;
(7) SEQ ID NOs: 87, 88, 89, 90, 91, and 92, respectively, or SEQ ID NOs: 93, 94, 95, 96, 97, and 98, respectively;
(8) SEQ ID NOs: 101, 102, 103, 104, 105, and 106, respectively, or SEQ ID NOs: 107, 108, 109, 110, 111, and 112, respectively;
(9) SEQ ID NOs: 115, 116, 117, 118, 119, and 120, respectively, or SEQ ID NOs: 121, 122, 123, 124, 125, and 126, respectively;
(10) SEQ ID NOs: 129, 130, 131, 132, 133, and 134, respectively, or SEQ ID NOs: 135, 136, 137, 138, 139, and 140, respectively;
(11) SEQ ID NOs: 143, 144, 145, 146, 147, and 148, respectively, or SEQ ID NOs: 149, 150, 151, 152, 153, and 154, respectively;
(12) SEQ ID NOs: 157, 158, 159, 160, 161, and 162, respectively, or SEQ ID NOs: 163, 164, 165, 166, 167, and 168, respectively; or
(13) SEQ ID NOs: 171, 172, 173, 174, 175, and 176, respectively, or SEQ ID NOs: 177, 178, 179, 180, 181, and 182, respectively;
wherein the antibody or antigen-binding fragment or antigen-binding domain thereof specifically binds IL13Ra2, preferably human IL13Ra2.

2. The isolated monoclonal antibody or antigen-binding fragment or the antigen-binding domain thereof of claim 1, comprising a heavy chain variable region having a polypeptide sequence at least 95% identical to SEQ ID NO: 1, 15, 29, 43, 57, 71, 85, 99, 113, 127, 141, 155, 169, 183, 184, 185, 186, 191, 192, 193, 197, 198, 199, 203, 204, 207, 208, 211, or 212, or a light chain variable region having a polypeptide sequence at least 95% identical to SEQ ID NO: 2, 16, 30, 44, 58, 72, 86, 100, 114, 128, 142, 156, 170, 187, 188, 189, 190, 194, 195, 196, 200, 201, 202, 205, 206, 209, 210, or 213.

3. The isolated monoclonal antibody or antigen-binding fragment or the antigen-binding domain thereof of claim 1, comprising:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:15, and a light chain variable region having the polypeptide sequence of SEQ ID NO:16;
(3) a heavy chain variable region having the polypeptide sequence of SEQ ID NO:29, and a light chain variable region having the polypeptide sequence of SEQ ID NO:30;
(4) a heavy chain variable region having the polypeptide sequence of SEQ ID NO:43, and a light chain variable region having the polypeptide sequence of SEQ ID NO:44;
(5) a heavy chain variable region having the polypeptide sequence of SEQ ID NO:57, and a light chain variable region having the polypeptide sequence of SEQ ID NO:58;
(6) a heavy chain variable region having the polypeptide sequence of SEQ ID NO:71, and a light chain variable region having the polypeptide sequence of SEQ ID NO:72;
(7) a heavy chain variable region having the polypeptide sequence of SEQ ID NO:85, and a light chain variable region having the polypeptide sequence of SEQ ID NO:86;
(8) a heavy chain variable region having the polypeptide sequence of SEQ ID NO:99, and a light chain variable region having the polypeptide sequence of SEQ ID NO:100;
(9) a heavy chain variable region having the polypeptide sequence of SEQ ID NO:113, and a light chain variable region having the polypeptide sequence of SEQ ID NO:114;
(10) a heavy chain variable region having the polypeptide sequence of SEQ ID NO:127, and a light chain variable region having the polypeptide sequence of SEQ ID NO:128;
(11) a heavy chain variable region having the polypeptide sequence of SEQ ID NO:141, and a light chain variable region having the polypeptide sequence of SEQ ID NO:142;
(12) a heavy chain variable region having the polypeptide sequence of SEQ ID NO:155, and a light chain variable region having the polypeptide sequence of SEQ ID NO:156;
(13) a heavy chain variable region having the polypeptide sequence of SEQ ID NO:169, and a light chain variable region having the polypeptide sequence of SEQ ID NO:170;
(14) a heavy chain variable region having the polypeptide sequence of SEQ ID NO:183, and a light chain variable region having the polypeptide sequence of SEQ ID NO:187;
(15) a heavy chain variable region having the polypeptide sequence of SEQ ID NO:183, and a light chain variable region having the polypeptide sequence of SEQ ID NO:188;
(16) a heavy chain variable region having the polypeptide sequence of SEQ ID NO:183, and a light chain variable region having the polypeptide sequence of SEQ ID NO:189;
(17) a heavy chain variable region having the polypeptide sequence of SEQ ID NO:183, and a light chain variable region having the polypeptide sequence of SEQ ID NO:190;
(18) a heavy chain variable region having the polypeptide sequence of SEQ ID NO:184, and a light chain variable region having the polypeptide sequence of SEQ ID NO:187;
(19) a heavy chain variable region having the polypeptide sequence of SEQ ID NO:184, and a light chain variable region having the polypeptide sequence of SEQ ID NO:188;
(20) a heavy chain variable region having the polypeptide sequence of SEQ ID NO:184, and a light chain variable region having the polypeptide sequence of SEQ ID NO:189;
(21) a heavy chain variable region having the polypeptide sequence of SEQ ID NO:184, and a light chain variable region having the polypeptide sequence of SEQ ID NO:190;
(22) a heavy chain variable region having the polypeptide sequence of SEQ ID NO:185, and a light chain variable region having the polypeptide sequence of SEQ ID NO:187;
(23) a heavy chain variable region having the polypeptide sequence of SEQ ID NO:185, and a light chain variable region having the polypeptide sequence of SEQ ID NO:188;
(24) a heavy chain variable region having the polypeptide sequence of SEQ ID NO:185, and a light chain variable region having the polypeptide sequence of SEQ ID NO:189;
(25) a heavy chain variable region having the polypeptide sequence of SEQ ID NO:185, and a light chain variable region having the polypeptide sequence of SEQ ID NO:190;
(26) a heavy chain variable region having the polypeptide sequence of SEQ ID NO:186, and a light chain variable region having the polypeptide sequence of SEQ ID NO:187;
(27) a heavy chain variable region having the polypeptide sequence of SEQ ID NO:186, and a light chain variable region having the polypeptide sequence of SEQ ID NO:188;
(28) a heavy chain variable region having the polypeptide sequence of SEQ ID NO:186, and a light chain variable region having the polypeptide sequence of SEQ ID NO:189;
(29) a heavy chain variable region having the polypeptide sequence of SEQ ID NO:186, and a light chain variable region having the polypeptide sequence of SEQ ID NO:190;
(30) a heavy chain variable region having the polypeptide sequence of SEQ ID NO:191, and a light chain variable region having the polypeptide sequence of SEQ ID NO:194;
(31) a heavy chain variable region having the polypeptide sequence of SEQ ID NO:191, and a light chain variable region having the polypeptide sequence of SEQ ID NO:195;
(32) a heavy chain variable region having the polypeptide sequence of SEQ ID NO:191, and a light chain variable region having the polypeptide sequence of SEQ ID NO:196;
(33) a heavy chain variable region having the polypeptide sequence of SEQ ID NO:192, and a light chain variable region having the polypeptide sequence of SEQ ID NO:194;
(34) a heavy chain variable region having the polypeptide sequence of SEQ ID NO:192, and a light chain variable region having the polypeptide sequence of SEQ ID NO:195;
(35) a heavy chain variable region having the polypeptide sequence of SEQ ID NO:192, and a light chain variable region having the polypeptide sequence of SEQ ID NO:196;
(36) a heavy chain variable region having the polypeptide sequence of SEQ ID NO:193, and a light chain variable region having the polypeptide sequence of SEQ ID NO:194;
(37) a heavy chain variable region having the polypeptide sequence of SEQ ID NO:193, and a light chain variable region having the polypeptide sequence of SEQ ID NO:195;
(38) a heavy chain variable region having the polypeptide sequence of SEQ ID NO:193, and a light chain variable region having the polypeptide sequence of SEQ ID NO:196;
(39) a heavy chain variable region having the polypeptide sequence of SEQ ID NO:197, and a light chain variable region having the polypeptide sequence of SEQ ID NO:200;
(40) a heavy chain variable region having the polypeptide sequence of SEQ ID NO:197, and a light chain variable region having the polypeptide sequence of SEQ ID NO:201;
(41) a heavy chain variable region having the polypeptide sequence of SEQ ID NO:197, and a light chain variable region having the polypeptide sequence of SEQ ID NO:202;
(42) a heavy chain variable region having the polypeptide sequence of SEQ ID NO:198, and a light chain variable region having the polypeptide sequence of SEQ ID NO:200;
(43) a heavy chain variable region having the polypeptide sequence of SEQ ID NO:198, and a light chain variable region having the polypeptide sequence of SEQ ID NO:201;
(44) a heavy chain variable region having the polypeptide sequence of SEQ ID NO:198, and a light chain variable region having the polypeptide sequence of SEQ ID NO:202;
(45) a heavy chain variable region having the polypeptide sequence of SEQ ID NO:199, and a light chain variable region having the polypeptide sequence of SEQ ID NO:200;
(46) a heavy chain variable region having the polypeptide sequence of SEQ ID NO:199, and a light chain variable region having the polypeptide sequence of SEQ ID NO:201;
(47) a heavy chain variable region having the polypeptide sequence of SEQ ID NO:199, and a light chain variable region having the polypeptide sequence of SEQ ID NO:202;
(48) a heavy chain variable region having the polypeptide sequence of SEQ ID NO:203, and a light chain variable region having the polypeptide sequence of SEQ ID NO:205;
(49) a heavy chain variable region having the polypeptide sequence of SEQ ID NO:203, and a light chain variable region having the polypeptide sequence of SEQ ID NO:206;
(50) a heavy chain variable region having the polypeptide sequence of SEQ ID NO:204, and a light chain variable region having the polypeptide sequence of SEQ ID NO:205;
(51) a heavy chain variable region having the polypeptide sequence of SEQ ID NO:204, and a light chain variable region having the polypeptide sequence of SEQ ID NO:206;
(52) a heavy chain variable region having the polypeptide sequence of SEQ ID NO:207, and a light chain variable region having the polypeptide sequence of SEQ ID NO:209;
(53) a heavy chain variable region having the polypeptide sequence of SEQ ID NO:207, and a light chain variable region having the polypeptide sequence of SEQ ID NO:210;
(54) a heavy chain variable region having the polypeptide sequence of SEQ ID NO:208, and a light chain variable region having the polypeptide sequence of SEQ ID NO:209;
(55) a heavy chain variable region having the polypeptide sequence of SEQ ID NO:208, and a light chain variable region having the polypeptide sequence of SEQ ID NO:210;
(56) a heavy chain variable region having the polypeptide sequence of SEQ ID NO:211, and a light chain variable region having the polypeptide sequence of SEQ ID NO:213; or
(57) a heavy chain variable region having the polypeptide sequence of SEQ ID NO:212, and a light chain variable region having the polypeptide sequence of SEQ ID NO:213.

4. The isolated monoclonal antibody or antigen-binding fragment or the antigen-binding domain thereof of claim 1, wherein the antibody or antigen-binding fragment or the antigen-binding domain thereof is chimeric or human or humanized.

5.-6. (canceled)

7. The isolated monoclonal antibody or antigen-binding fragment or the antigen-binding domain thereof of claim 1, wherein the antibody or antigen-binding fragment or the antigen-binding domain thereof is capable of inducing effector-mediated tumor cell lysis through antibody-dependent cellular cytotoxicity (ADCC), antibody-dependent cellular phagocytosis (ADCP), and/or complement-dependent cytotoxicity (CDC), and/or mediating the recruitment of conjugated drugs, and/or forming a bispecific antibody with another mAb or antigen-binding fragment thereof with cancer-killing effect.

8. A bispecific antibody or antigen-binding fragment or a bispecific antigen-binding domain thereof comprising the monoclonal antibody or antigen-binding fragment or the antigen-binding domain thereof of claim 1.

9. An isolated nucleic acid encoding the monoclonal antibody or antigen-binding fragment or the antigen-binding domain thereof of claim 1.

10. A vector comprising the isolated nucleic acid of claim 9.

11. A host cell comprising the vector of claim 10.

12. A pharmaceutical composition, comprising the isolated monoclonal antibody or antigen-binding fragment or the antigen-binding domain thereof of claim 1.

13. A method of targeting IL13Ra2 on a cancer cell surface, and/or treating a cancer, and/or treating an inflammatory disease, and/or treating an autoimmune disease in a subject in need thereof, comprising administering to the subject the pharmaceutical composition of claim 12.

14. A method of producing the monoclonal antibody or antigen-binding fragment or the antigen-binding domain thereof of claim 1, comprising culturing a cell comprising a nucleic acid encoding the monoclonal antibody or antigen-binding fragment or the antigen-binding domain thereof under conditions to produce the monoclonal antibody or antigen-binding fragment or the antigen-binding domain thereof and recovering the monoclonal antibody or antigen-binding fragment or the antigen-binding domain thereof from the cell or culture.

15. A method of producing a pharmaceutical composition comprising the monoclonal antibody or antigen-binding fragment or the antigen-binding domain thereof of claim 1, comprising combining the monoclonal antibody or antigen-binding fragment or the antigen-binding domain thereof with a pharmaceutically acceptable carrier to obtain the pharmaceutical composition.

16. A method of determining the level of IL13Ra2 in a subject, the method comprising:
(a) obtaining a sample from the subject;
(b) contacting the sample with an isolated monoclonal antibody or antigen-binding fragment or the antigen-binding domain thereof of claim 1; and
(c) determining the level of a IL13Ra2 in the subject.

17. The method of claim 16, wherein the sample is a tissue sample or a blood sample.

18. An isolated polynucleotide comprising a nucleic acid sequence encoding a chimeric antigen receptor (CAR), wherein the CAR comprises:
(a) an extracellular domain comprising at least one antigen binding domain thereof of claim 1;
(b) a hinge region;
(c) a transmembrane region; and
(d) an intracellular signaling domain.

19.-24. (canceled)

25. A chimeric antigen receptor (CAR) encoded by the isolated polynucleotide of claim 18.

26. (canceled)

27. A host cell comprising a vector comprising the isolated polynucleotide of claim 18.

28.-32. (canceled)

33. The method of claim 13, wherein the cancer is selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a mesothelioma, and other solid tumors, and a hon-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

34. The host cell of claim 27, wherein the host cell is a T cell or a NK cell.


SEQUENCE LISTING (213 sequences)

SEQ ID NO: 1 - protein, 119 aa, synthetic construct
QVQLQQPGAELVKPGAPVKLSCKASGYTFTNYWMNWVKQRPGRGLEWIGRIDPSDTETHYNQEVKDKATLTVDKSSSTAYIQLSSLTSEDSAVYYCARGYGTYYGMDYWGQGTSVTVSS

SEQ ID NO: 2 - protein, 111 aa, synthetic construct
DIVLTQSPASLAVSLGQRATISCRASESVDNYGISFMNWFQQKPGQPPKLLIYAASNQGSGVPARFSGSGSGTDFSLNIHPLEEDDTALYFCQQSKEVPWTFGGGTKLEIK

SEQ ID NO: 3 - protein, 8 aa, synthetic construct
GYTFTNYW

SEQ ID NO: 4 - protein, 8 aa, synthetic construct
IDPSDTET

SEQ ID NO: 5 - protein, 12 aa, synthetic construct
ARGYGTYYGMDY

SEQ ID NO: 6 - protein, 10 aa, synthetic construct
ESVDNYGISF

SEQ ID NO: 7 - skipped

SEQ ID NO: 8 - protein, 9 aa, synthetic construct
QQSKEVPWT

SEQ ID NO: 9 - protein, 5 aa, synthetic construct
NYWMN

SEQ ID NO: 10 - protein, 17 aa, synthetic construct
RIDPSDTETHYNQEVKD

SEQ ID NO: 11 - protein, 10 aa, synthetic construct
GYGTYYGMDY

SEQ ID NO: 12 - protein, 15 aa, synthetic construct
RASESVDNYGISFMN

SEQ ID NO: 13 - protein, 7 aa, synthetic construct
AASNQGS

SEQ ID NO: 14 - protein, 9 aa, synthetic construct
QQSKEVPWT

SEQ ID NO: 15 - protein, 120 aa, synthetic construct
QVQLQQSGPELVKPGASVRLSCKASGYSFTSYYIHWMKQRPGQGLEWIGYIFPGDGSSNYNEKFKGKATLTADTSSSTVYMQLSSLTSEDSAVYFCARPYYYSGRGFAYWGQGTLVTVSA

SEQ ID NO: 16 - protein, 107 aa, synthetic construct
DIVLTQSPATLSVTPGDRVSLSCRASQSISNYLHWYQQKSHESPRLLIKSASQSISGIPSRFSGSGSGTDFTLSINSVETEDFGMYFCQQSNSWPLTFGAGTKLELK

SEQ ID NO: 17 - protein, 8 aa, synthetic construct
GYSFTSYY

SEQ ID NO: 18 - protein, 8 aa, synthetic construct
IFPGDGSS

SEQ ID NO: 19 - protein, 13 aa, synthetic construct
ARPYYYSGRGFAY

SEQ ID NO: 20 - protein, 6 aa, synthetic construct
QSISNY

SEQ ID NO: 21 - skipped

SEQ ID NO: 22 - protein, 9 aa, synthetic construct
QQSNSWPLT

SEQ ID NO: 23 - protein, 5 aa, synthetic construct
SYYIH

SEQ ID NO: 24 - protein, 17 aa, synthetic construct
YIFPGDGSSNYNEKFKG

SEQ ID NO: 25 - protein, 11 aa, synthetic construct
PYYYSGRGFAY

SEQ ID NO: 26 - protein, 11 aa, synthetic construct
RASQSISNYLH

SEQ ID NO: 27 - protein, 7 aa, synthetic construct
SASQSIS

SEQ ID NO: 28 - protein, 9 aa, synthetic construct
QQSNSWPLT

SEQ ID NO: 29 - protein, 118 aa, synthetic construct
QVQLQQSGPELVKPGASVKISCKASGYVFSRSWMNWVKQRPGKGLEWIGRIYPGDGDTNYNGKFKGKATLTADKSSSTAYMQLSSLTSEDSAVYFCARGYYGNYFDYWGQGTTLTVSS

SEQ ID NO: 30 - protein, 112 aa, synthetic construct
DIVMTQAAFSNPVTLGTSASISCRSSKSLLHTDGITYLYWYLQRPGQSPQLLIYWMSNLASGVPDRFSGSGSGTDFTLRISRVEAEDVGVYYCAQILEFPRTFGGGTKLEIK

SEQ ID NO: 31 - protein, 8 aa, synthetic construct
GYVFSRSW

SEQ ID NO: 32 - protein, 8 aa, synthetic construct
IYPGDGDT

SEQ ID NO: 33 - protein, 11 aa, synthetic construct
ARGYYGNYFDY

SEQ ID NO: 34 - protein, 11 aa, synthetic construct
KSLLHTDGITY

SEQ ID NO: 35 - skipped

SEQ ID NO: 36 - protein, 9 aa, synthetic construct
AQILEFPRT

SEQ ID NO: 37 - protein, 5 aa, synthetic construct
RSWMN

SEQ ID NO: 38 - protein, 17 aa, synthetic construct
RIYPGDGDTNYNGKFKG

SEQ ID NO: 39 - protein, 9 aa, synthetic construct
GYYGNYFDY

SEQ ID NO: 40 - protein, 16 aa, synthetic construct
RSSKSLLHTDGITYLY

SEQ ID NO: 41 - protein, 7 aa, synthetic construct
WMSNLAS

SEQ ID NO: 42 - protein, 9 aa, synthetic construct
AQILEFPRT

SEQ ID NO: 43 - protein, 118 aa, synthetic construct
QAQLQQSGAELVKPGASVKISCKASGYAFSDSWMSWVKQRPGKGLEWIGQIYPGDGDTNFNGNFKGKATLTADKSSSTAYMHLSSLTSEDSAVYFCARRYYPVWFAYWGQGTLVTVSA

SEQ ID NO: 44 - protein, 112 aa, synthetic construct
DVLMTQTPLSLPVSLGDQASISCRSSQSIVHSNGNTYLEWYLQKPGQSPKPLIYKVSNRFSGVPDRFSGSGSGTDFTLKISRVEAEDLGVYYCFQSSHVPFTFGSGTKLEIK

SEQ ID NO: 45 - protein, 8 aa, synthetic construct
GYAFSDSW

SEQ ID NO: 46 - protein, 8 aa, synthetic construct
IYPGDGDT

SEQ ID NO: 47 - protein, 11 aa, synthetic construct
ARRYYPVWFAY

SEQ ID NO: 48 - protein, 11 aa, synthetic construct
QSIVHSNGNTY

SEQ ID NO: 49 - skipped

SEQ ID NO: 50 - protein, 9 aa, synthetic construct
FQSSHVPFT

SEQ ID NO: 51 - protein, 5 aa, synthetic construct
DSWMS

SEQ ID NO: 52 - protein, 17 aa, synthetic construct
QIYPGDGDTNFNGNFKG

SEQ ID NO: 53 - protein, 9 aa, synthetic construct
RYYPVWFAY

SEQ ID NO: 54 - protein, 16 aa, synthetic construct
RSSQSIVHSNGNTYLE

SEQ ID NO: 55 - protein, 7 aa, synthetic construct
KVSNRFS

SEQ ID NO: 56 - protein, 9 aa, synthetic construct
FQSSHVPFT

SEQ ID NO: 57 - protein, 123 aa, synthetic construct
QVTLKESGPGILQSSQTLSLTCSFSGFSLSTSGMGVSWIRQPSGMGLEWLAHIYWDDDKRYNPSLKSRLTISKDTSRDQIFLKIASVDTSDTATYYCARRAYDNYPYYYFDVWGAGTTVTVSS

SEQ ID NO: 58 - protein, 106 aa, synthetic construct
DIQMTQTTSSLSASLGDRVTISCRASQDISNYLNWYQQKPDGTVKLLIYYTSRLHSGVPSRFSGSGSGTDYSLTISNLEPEDIATYYCQQYGKLFTFGTGTKLEIK

SEQ ID NO: 59 - protein, 10 aa, synthetic construct
GFSLSTSGMG

SEQ ID NO: 60 - protein, 7 aa, synthetic construct
IYWDDDK

SEQ ID NO: 61 - protein, 15 aa, synthetic construct
ARRAYDNYPYYYFDV

SEQ ID NO: 62 - protein, 6 aa, synthetic construct
QDISNY

SEQ ID NO: 63 - skipped

SEQ ID NO: 64 - protein, 8 aa, synthetic construct
QQYGKLFT

SEQ ID NO: 65 - protein, 7 aa, synthetic construct
TSGMGVS

SEQ ID NO: 66 - protein, 16 aa, synthetic construct
HIYWDDDKRYNPSLKS

SEQ ID NO: 67 - protein, 13 aa, synthetic construct
RAYDNYPYYYFDV

SEQ ID NO: 68 - protein, 11 aa, synthetic construct
RASQDISNYLN

SEQ ID NO: 69 - protein, 7 aa, synthetic construct
YTSRLHS

SEQ ID NO: 70 - protein, 8 aa, synthetic construct
QQYGKLFT

SEQ ID NO: 71 - protein, 119 aa, synthetic construct
EVQLQQSGPALVKPGASVKISCKASGFTFTDYYMDWVKQSHGKSLEWIGYIYPNNGGTNYNRKFKDKASLTVDKSSSTAYMELRSLTSEDSAVYYCARRYGNYIWFAYWGQGTLVTVSA

SEQ ID NO: 72 - protein, 111 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPTFGGGTKLEIK

SEQ ID NO: 73 - protein, 8 aa, synthetic construct
GFTFTDYY

SEQ ID NO: 74 - protein, 8 aa, synthetic construct
IYPNNGGT

SEQ ID NO: 75 - protein, 12 aa, synthetic construct
ARRYGNYIWFAY

SEQ ID NO: 76 - protein, 11 aa, synthetic construct
QSLVHSNGNTY

SEQ ID NO: 77 - skipped

SEQ ID NO: 78 - protein, 8 aa, synthetic construct
SQSTHVPT

SEQ ID NO: 79 - protein, 5 aa, synthetic construct
DYYMD

SEQ ID NO: 80 - protein, 17 aa, synthetic construct
YIYPNNGGTNYNRKFKD

SEQ ID NO: 81 - protein, 10 aa, synthetic construct
RYGNYIWFAY

SEQ ID NO: 82 - protein, 16 aa, synthetic construct
RSSQSLVHSNGNTYLH

SEQ ID NO: 83 - protein, 7 aa, synthetic construct
KVSNRFS

SEQ ID NO: 84 - protein, 8 aa, synthetic construct
SQSTHVPT

SEQ ID NO: 85 - protein, 119 aa, synthetic construct
QVQLQQSGPELVKPGASVKISCKASGYSFTSYYIHWVKQRPGQGLEWIGWIYPGSDNTKYNEKFKDKATLTADTSSSTAYMQLSSLTSEDSAVYYCARDWENRPYFGYWGQGTTLTVSS

SEQ ID NO: 86 - protein, 107 aa, synthetic construct
DIVLIQSPATLSVTPGDRVSLSCRASQSISSYLHWYQQKSHESPRLLIKYASQSISGIPSRFSGSGSGTDFTLSINSVETEDFGMYFCQQSNSWPLTFGAGTKLVLK

SEQ ID NO: 87 - protein, 8 aa, synthetic construct
GYSFTSYY

SEQ ID NO: 88 - protein, 8 aa, synthetic construct
IYPGSDNT

SEQ ID NO: 89 - protein, 12 aa, synthetic construct
ARDWENRPYFGY

SEQ ID NO: 90 - protein, 6 aa, synthetic construct
QSISSY

SEQ ID NO: 91 - skipped

SEQ ID NO: 92 - protein, 9 aa, synthetic construct
QQSNSWPLT

SEQ ID NO: 93 - protein, 5 aa, synthetic construct
SYYIH

SEQ ID NO: 94 - protein, 17 aa, synthetic construct
WIYPGSDNTKYNEKFKD

SEQ ID NO: 95 - protein, 10 aa, synthetic construct
DWENRPYFGY

SEQ ID NO: 96 - protein, 11 aa, synthetic construct
RASQSISSYLH

SEQ ID NO: 97 - protein, 7 aa, synthetic construct
YASQSIS

SEQ ID NO: 98 - protein, 9 aa, synthetic construct
QQSNSWPLT

SEQ ID NO: 99 - protein, 120 aa, synthetic construct
QVQLKQSGAELVRPGASVKLSCKASGYTFTDSYINWVRQRPGQGLEWIARIYPKNNNTYYNEKFKDKATLTAEKSSSTAYMQLSSLTSEDSAVYFCARTGGSSWAWFAYWGQGTLVTVSA

SEQ ID NO: 100 - protein, 107 aa, synthetic construct
DIQMTQSPASLAASVGETITITCQASENIYFSLAWYQQKQGKSPQLLIYNANSLEDGVPSRFSGSGSGTQFSMKINSMQPEDTATYFCKQAYDFPYTFGGGTKLEIK

SEQ ID NO: 101 - protein, 8 aa, synthetic construct
GYTFTDSY

SEQ ID NO: 102 - protein, 8 aa, synthetic construct
IYPKNNNT

SEQ ID NO: 103 - protein, 13 aa, synthetic construct
ARTGGSSWAWFAY

SEQ ID NO: 104 - protein, 6 aa, synthetic construct
ENIYFS

SEQ ID NO: 105 - skipped

SEQ ID NO: 106 - protein, 9 aa, synthetic construct
KQAYDFPYT

SEQ ID NO: 107 - protein, 5 aa, synthetic construct
DSYIN

SEQ ID NO: 108 - protein, 17 aa, synthetic construct
RIYPKNNNTYYNEKFKD

SEQ ID NO: 109 - protein, 11 aa, synthetic construct
TGGSSWAWFAY

SEQ ID NO: 110 - protein, 11 aa, synthetic construct
QASENIYFSLA

SEQ ID NO: 111 - protein, 7 aa, synthetic construct
NANSLED

SEQ ID NO: 112 - protein, 9 aa, synthetic construct
KQAYDFPYT

SEQ ID NO: 113 - protein, 116 aa, synthetic construct
EVKVLESGGGLVQPGGSLKLSCAASGFDFSKAWMSWVRQATGKGLEWIGEISPGSSTINYTPSLKDKFIISRDNAKNTLYLQMSKVRSEDTALYYCSSDYDPFAFWGQGILVTVSA

SEQ ID NO: 114 - protein, 112 aa, synthetic construct
DVLMTQTPLSLPVSLGDHASISCRSSQTIVHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 115 - protein, 8 aa, synthetic construct
GFDFSKAW

SEQ ID NO: 116 - protein, 8 aa, synthetic construct
ISPGSSTI

SEQ ID NO: 117 - protein, 9 aa, synthetic construct
SSDYDPFAF

SEQ ID NO: 118 - protein, 11 aa, synthetic construct
QTIVHSNGNTY

SEQ ID NO: 119 - skipped

SEQ ID NO: 120 - protein, 9 aa, synthetic construct
FQGSHVPFT

SEQ ID NO: 121 - protein, 5 aa, synthetic construct
KAWMS

SEQ ID NO: 122 - protein, 17 aa, synthetic construct
EISPGSSTINYTPSLKD

SEQ ID NO: 123 - protein, 7 aa, synthetic construct
DYDPFAF

SEQ ID NO: 124 - protein, 16 aa, synthetic construct
RSSQTIVHSNGNTYLE

SEQ ID NO: 125 - protein, 7 aa, synthetic construct
KVSNRFS

SEQ ID NO: 126 - protein, 9 aa, synthetic construct
FQGSHVPFT

SEQ ID NO: 127 - protein, 122 aa, synthetic construct
EVQLQQSGAELVKPGASVKLSCTASGFNIKDYYMNWVKQRTEQGLEWIGRIDPEDGEIKYAPKFQGKATITADTSSNTAYLQVSSLTSEDTAVYYCARPRDSSGYVGWFAYWGQGTLVTVSA

SEQ ID NO: 128 - protein, 108 aa, synthetic construct
QIVLTQSPAIMSASPGEKVTMTCSASSTVSSSYLHWYQQKSGASPKLWIYGTSNLASGVPARFSGSGSGTSYSLTISGVEAEDAATYYCQQYHSDPPTFGGGTKLEIK

SEQ ID NO: 129 - protein, 8 aa, synthetic construct
GFNIKDYY

SEQ ID NO: 130 - protein, 8 aa, synthetic construct
IDPEDGEI

SEQ ID NO: 131 - protein, 15 aa, synthetic construct
ARPRDSSGYVGWFAY

SEQ ID NO: 132 - protein, 7 aa, synthetic construct
STVSSSY

SEQ ID NO: 133 - skipped

SEQ ID NO: 134 - protein, 9 aa, synthetic construct
QQYHSDPPT

SEQ ID NO: 135 - protein, 5 aa, synthetic construct
DYYMN

SEQ ID NO: 136 - protein, 17 aa, synthetic construct
RIDPEDGEIKYAPKFQG

SEQ ID NO: 137 - protein, 13 aa, synthetic construct
PRDSSGYVGWFAY

SEQ ID NO: 138 - protein, 12 aa, synthetic construct
SASSTVSSSYLH

SEQ ID NO: 139 - protein, 7 aa, synthetic construct
GTSNLAS

SEQ ID NO: 140 - protein, 9 aa, synthetic construct
QQYHSDPPT

SEQ ID NO: 141 - protein, 120 aa, synthetic construct
QVQLQQSGAELMKPGASVKLSCKATGYTFTGYWIEWVKERPGHGLEWIGEILPGNGNTYYNEKFKGKATFTADTSSNTAYMLLSSLTTEDSAIYYCASERRNRYAWFAYWGQGTLVTVSA

SEQ ID NO: 142 - protein, 112 aa, synthetic construct
DVVMTQTPLSLSVTIGQPASISCKSSQSLLYSDGKTYLTWLQQRPGQSPKRLMYQVSKLDPGIPDRFSGSGSETDFSLKISRVEAEDLGVYYCLQGTYYPRTFGGGTKLEIK

SEQ ID NO: 143 - protein, 8 aa, synthetic construct
GYTFTGYW

SEQ ID NO: 144 - protein, 8 aa, synthetic construct
ILPGNGNT

SEQ ID NO: 145 - protein, 13 aa, synthetic construct
ASERRNRYAWFAY

SEQ ID NO: 146 - protein, 11 aa, synthetic construct
QSLLYSDGKTY

SEQ ID NO: 147 - skipped

SEQ ID NO: 148 - protein, 9 aa, synthetic construct
LQGTYYPRT

SEQ ID NO: 149 - protein, 5 aa, synthetic construct
GYWIE

SEQ ID NO: 150 - protein, 17 aa, synthetic construct
EILPGNGNTYYNEKFKG

SEQ ID NO: 151 - protein, 11 aa, synthetic construct
ERRNRYAWFAY

SEQ ID NO: 152 - protein, 16 aa, synthetic construct
KSSQSLLYSDGKTYLT

SEQ ID NO: 153 - protein, 7 aa, synthetic construct
QVSKLDP

SEQ ID NO: 154 - protein, 9 aa, synthetic construct
LQGTYYPRT

SEQ ID NO: 155 - protein, 122 aa, synthetic construct
EVQLQQSGAELVKPGASVKLSCTASGFNIKDYYMNWVKQRTEQGLEWIGRIDPEDGETKYAPKFQGRATITADTSSNTAYLQLGSLTSEDTAVYYCARPLASSGYVGWFAYWGQGTLVTVSS

SEQ ID NO: 156 - protein, 108 aa, synthetic construct
QIVLTQSPAIMSASPGEKVTMTCSASSSVSSRYLHWYQQKSGASPKLWIYVTSNLASGVPARFSGSGSGTSYSLTISSVEAEDAATYYCQQYHSAPPTFGGGTKLEIK

SEQ ID NO: 157 - protein, 8 aa, synthetic construct
GFNIKDYY

SEQ ID NO: 158 - protein, 8 aa, synthetic construct
IDPEDGET

SEQ ID NO: 159 - protein, 15 aa, synthetic construct
ARPLASSGYVGWFAY

SEQ ID NO: 160 - protein, 7 aa, synthetic construct
SSVSSRY

SEQ ID NO: 161 - skipped

SEQ ID NO: 162 - protein, 9 aa, synthetic construct
QQYHSAPPT

SEQ ID NO: 163 - protein, 5 aa, synthetic construct
DYYMN

SEQ ID NO: 164 - protein, 17 aa, synthetic construct
RIDPEDGETKYAPKFQG

SEQ ID NO: 165 - protein, 13 aa, synthetic construct
PLASSGYVGWFAY

SEQ ID NO: 166 - protein, 12 aa, synthetic construct
SASSSVSSRYLH

SEQ ID NO: 167 - protein, 7 aa, synthetic construct
VTSNLAS

SEQ ID NO: 168 - protein, 9 aa, synthetic construct
QQYHSAPPT

SEQ ID NO: 169 - protein, 121 aa, synthetic construct
QVQLQQSGAELMKPGASVKLSCKATGYTFTGYWIEWLKQRPGHGLEWIGEILPGNGSTYYNEKFKGKATFTADTSSNTAYMQLSSLTTEDSAIYYCATEEYGNYEAWFSYWGQGTLVTVSA

SEQ ID NO: 170 - protein, 112 aa, synthetic construct
DVVMTQTPLSLSVTIGQPASISCKSSQSLLYSDGKTYLNWLQQRPGQSPKRLMYQVSKLDPGIPDRFSGSGSETDFTLKISRVEAEDLGVYYCLQGTYYPQTFGSGTKLEIK

SEQ ID NO: 171 - protein, 8 aa, synthetic construct
GYTFTGYW

SEQ ID NO: 172 - protein, 8 aa, synthetic construct
ILPGNGST

SEQ ID NO: 173 - protein, 14 aa, synthetic construct
ATEEYGNYEAWFSY

SEQ ID NO: 174 - protein, 11 aa, synthetic construct
QSLLYSDGKTY

SEQ ID NO: 175 - skipped

SEQ ID NO: 176 - protein, 9 aa, synthetic construct
LQGTYYPQT

SEQ ID NO: 177 - protein, 5 aa, synthetic construct
GYWIE

SEQ ID NO: 178 - protein, 17 aa, synthetic construct
EILPGNGSTYYNEKFKG

SEQ ID NO: 179 - protein, 12 aa, synthetic construct
EEYGNYEAWFSY

SEQ ID NO: 180 - protein, 16 aa, synthetic construct
KSSQSLLYSDGKTYLN

SEQ ID NO: 181 - protein, 7 aa, synthetic construct
QVSKLDP

SEQ ID NO: 182 - protein, 9 aa, synthetic construct
LQGTYYPQT

SEQ ID NO: 183 - protein, 119 aa, synthetic construct
QVTLRESGPALVKPTQTLTLTCTASGYTFTNYWMNWVRQPPGKALEWIGRIDPSDTETHYNQEVKDRATLSVDKSKNQAVLTMTNMDPVDTATYYCARGYGTYYGMDYWGQGSLVTVSS

SEQ ID NO: 184 - protein, 119 aa, synthetic construct
EVTLKESGPTLVKPTQTLTLTCTASGYTFTNYWMNWVRQPPGKALEWIGRIDPSDTETHYNQEVKDRATLTVDKSKNQAVLTMTNMDPVDTATYYCARGYGTYYGMDYWGQGTLVTVSS

SEQ ID NO: 185 - protein, 119 aa, synthetic construct
EVTLKESGPTLVKPTQTLTLTCKASGYTFTNYWMNWVRQPPGKALEWIGRIDPSDTETHYNQEVKDRATLTVDTSKNQAVLTMTNMDPVDTATYYCARGYGTYYGMDYWGQGTLVTVSS

SEQ ID NO: 186 - protein, 119 aa, synthetic construct
EVQLVQSGAELKKPGSSVKVSCKASGYTFTNYWMNWVKQAPGQGLEWIGRIDPSDTETHYNQEVKDKATLTVDKSTNTAYMELSSLRSEDTAVYYCARGYGTYYGMDYWGQGTLVTVSS

SEQ ID NO: 187 - protein, 111 aa, synthetic construct
DIVLTQSPDSLSVSLGERATINCRASESVDNYGISFMNWFQQKPGQPPKLLIYAASNQGSGVPDRFSGSGSGTDFTLTISSLQAEDVAVYFCQQSKEVPWTFGGGTKVEIK

SEQ ID NO: 188 - protein, 111 aa, synthetic construct
DIVLTQSPDSLAVSLGERATINCRASESVDNYGISFMNWFQQKPGQPPKLLIYAASNQGSGVPDRFSGSGSGTDFTLTISSLQAEDVAVYFCQQSKEVPWTFGQGTKVEIK

SEQ ID NO: 189 - protein, 111 aa, synthetic construct
DIVLTQSPASLAVSLGERATINCRASESVDNYGISFMNWFQQKPGQPPKLLIYAASNQGSGVPDRFSGSGSGTDFTLTISSLQAEDVAVYFCQQSKEVPWTFGQGTKVEIK

SEQ ID NO: 190 - protein, 111 aa, synthetic construct
DIQLTQSPSTLSASVGDRVTITCRASESVDNYGISFMNWFQQKPGKAPKLLIYAASNQGSGVPSRFSGSGSGTDFTLTISSLQPDDFATYFCQQSKEVPWTFGQGTKVEVK

SEQ ID NO: 191 - protein, 118 aa, synthetic construct
QAQLVQSGPEVKKPGTSVRVSCKASGYAFSDSWMSWVRQARGQRLEWIGQIYPGDGDTNFNGNFKGRATLTADKSTSTAYMELSSLRSEDTAVYFCARRYYPVWFAYWGQGTMVTVSS

SEQ ID NO: 192 - protein, 118 aa, synthetic construct
EVQLLESGGGLVQPGGSLRLSCAASGYAFSDSWMSWVRQAPGKGLEWIGQIYPGDGDTNFNGNFKGRATLSADKSKNTAYLQMNSLRAEDTAVYFCARRYYPVWFAYWGQGTMVTVSS

SEQ ID NO: 193 - protein, 118 aa, synthetic construct
QVQLEQSGAEVKKPGSSVKVSCKASGYAFSDSWMSWVRQAPGQGLEWIGQIYPGDGDTNFNQRFQGRATLTADKSMTTAYMELSSLRSEDTAMYFCARRYYPVWFAYWGQGTMVTVSS

SEQ ID NO: 194 - protein, 112 aa, synthetic construct
DVVMTQTPGTLSLSPGERATLSCRSSQSIVHSNGNTYLEWYQQKPGQAPRPLIYKVSNRFSGIPDRFSGSGSGTDFTLTISRLEPEDFAVYYCFQSSHVPFTFGQGTKVEIK

SEQ ID NO: 195 - protein, 112 aa, synthetic construct
DVQMTQSPSTLSASVGDRVTITCRSSQSIVHSNGNTYLEWYQQKPGKAPKPLIYKVSNRFSGVPSRFSGSGSGTEFTLTISSLQPDDFATYYCFQSSHVPFTFGQGTKVEIK

SEQ ID NO: 196 - protein, 112 aa, synthetic construct
EVEMTQSPATLSVSPGESATLSCRSSQSIVHSNGNTYLEWYQQKPGQAPRPLIYKVSNRAPAIPGRFSGSGSGTDFTLTISRLEPEDLAVYYCFQSSHVPFTFGQGTKVEIK

SEQ ID NO: 197 - protein, 118 aa, synthetic construct
QVQLQESGPGLVKPSETLSLTCTASGYVFSRSWMNWVRQSPGKGLEWIGRIYPGDGDTNYNGKFKGRATLSADKSKTQASLKLSSVTAADTAIYFCARGYYGNYFDYWGQGTMVTVSS

SEQ ID NO: 198 - protein, 118 aa, synthetic construct
QVQLVESGPGLVKPSQTLSLTCTASGYVFSRSWMNWVRQPAGKGLEWIGRIYPGDGDTNYNGKFKGRATLSADKSKNQASLKLSSVTAADTAVYFCARGYYGNYFDYWGKGTLVTVSS

SEQ ID NO: 199 - protein, 118 aa, synthetic construct
EVTLKESGPVLVKPTETLTLTCTASGYVFSRSWMNWVRQPPGKALEWIGRIYPGDGDTNYNGKFKGRATLSADKSKSQAVLTMTNMDPVDTATYFCARGYYGNYFDYWGQGTLVTVSS

SEQ ID NO: 200 - protein, 112 aa, synthetic construct
DIQMTQSPSSLSASVGDRVTITCRSSKSLLHTDGITYLYWYQQKPGKAPKLLIYWMSNLASGVPSRFSGSGSGTDFTFTISSLQPEDIATYYCAQILEFPRTFGGGTKVEIK

SEQ ID NO: 201 - protein, 112 aa, synthetic construct
DIVMTQSPSSLSASVGDRVTITCRSSKSLLHTDGITYLYWYQQKPGRAPDLLIYWMSNLASGVPSRFSGSGSGTDFTLTISSLQPEDFAVYYCAQILEFPRTFGQGTKLQIK

SEQ ID NO: 202 - protein, 112 aa, synthetic construct
EIVMTQSPATLSLSPGERATLSCRSSKSLLHTDGITYLYWYQQKPGQAPRLLIYWMSNLASGIPARFSGSGSGTDFTLTISSLEPEDFAVYYCAQILEFPRTFGQGTKLEIK

SEQ ID NO: 203 - protein, 123 aa, synthetic construct
QVQLQESGPGLVKPSETLSLTCTFSGFSLSTSGMGVSWIRQSPGKGLEWLAHIYWDDDKRYNPSLKSRLTISKDTSKTQISLKLSSVTAADTAIYYCARRAYDNYPYYYFDVWGQGTMVTVSS

SEQ ID NO: 204 - protein, 123 aa, synthetic construct
QVTLRESGPALVKPTQTLTLTCTFSGFSLSTSGMGVSWIRQPPGKALEWLAHIYWDDDKRYNPSLKSRLTISKDTSKNQIVLTMTNMDPVDTATYYCARRAYDNYPYYYFDVWGQGSLVTVSS

SEQ ID NO: 205 - protein, 106 aa, synthetic construct
DIQMTQSPSSLSASVGDRVTITCRASQDISNYLNWYQQKPGKAVKLLIYYTSRLHSGVPSRFSGSGSGTDYTFTISSLQPEDIATYYCQQYGKLFTFGGGTKVEIK

SEQ ID NO: 206 - protein, 106 aa, synthetic construct
DIVMTQSPDSLSVSLGERATINCRASQDISNYLNWYQQKPGQPVKLLIYYTSRLHSGVPDRFSGSGSGTDYTLTISSLQAEDVAVYYCQQYGKLFTFGGGTKVEIK

SEQ ID NO: 207 - protein, 119 aa, synthetic construct
EVTLKESGPTLVKPTQTLTLTCTASGFTFTDYYMDWVRQPPGKALEWIGYIYPNNGGTNYNRKFKDRATLTVDKSKNQAVLTMTNMDPVDTATYYCARRYGNYIWFAYWGQGTLVTVSS

SEQ ID NO: 208 - protein, 119 aa, synthetic construct
QVQLQESGPGLVKPSQTLSLTCTASGFTFTDYYMDWVRQLPGKGLEWIGYIYPNNGGTNYNRKFKDRATLSVDKSKKQASLRLSSVTAADTAVYYCARRYGNYIWFAYWGQGTTVTVSS

SEQ ID NO: 209 - protein, 111 aa, synthetic construct
DVVMTQSPDSLAVSLGERATINCRSSQSLVHSNGNTYLHWYQQKPGQPPKLLIYKVSNRFSGVPDRFSGSGSGTDFTLTISSLQAEDVAVYFCSQSTHVPTFGQGTKVEIK

SEQ ID NO: 210 - protein, 111 aa, synthetic construct
EVVMTQSPGTLSLSPGERATLSCRSSQSLVHSNGNTYLHWYQQKPGQAPSLLIYKVSNRFSGIPDRFSGSGSGTDFTLTISRLEPEDFAVYFCSQSTHVPTFGQGTKVEIK

SEQ ID NO: 211 - protein, 119 aa, synthetic construct
QVQLVQSGAEVKKPGSSVKVSCKASGYSFTSYYIHWVRQAPGQGLEWIGWIYPGSDNTKYNEKFKDRATLTADTSTSTAYMELSSLRSEDTAVYYCARDWENRPYFGYWGQGTLVTVSS

SEQ ID NO: 212 - protein, 119 aa, synthetic construct
QVQLEESGPGLVKPSETLSLTCSASGYSFTSYYIHWVRQPPGKGLEWIGWIYPGSDNTKYNEKFKDRATLSADTSKNEASLRLTSVTAADTAVYYCARDWENRPYFGYWGQGTLVTVSS

SEQ ID NO: 213 - protein, 107 aa, synthetic construct
DIQLTQSPSSVSASVGDRVTITCRASQSISSYLHWYQQKPGKAPKLLIKYASQSISGVPSRFSGSGSGTDFTLTISSLQPEDFATYFCQQSNSWPLTFGQGTKVEIK